(19) United States
(12) Patent Application Publication
(10) Pub. No.: US 2024/0084297 A1
(43) Pub. Date: Mar. 14, 2024

(54) CONJUGATED OLIGONUCLEOTIDES

(71) Applicant: UNIVERSITY OF MASSACHUSETTS, Boston, MA (US)

(72) Inventors: Anastasia KHVOROVA, Westborough, MA (US); Mehran NIKAN, Boston, MA (US); Matthew HASSLER, Worcester, MA (US); Maire OSBORN, Worcester, MA (US); Reka HARASZTI, Shrewsbury, MA (US); Andrew COLES, Auburn, MA (US); Anton TURANOV, Boston, MA (US); Neil ARONIN, Newtonville, MA (US); Annabelle BISCANS, Cambridge, MA (US)

(21) Appl. No.: 18/170,167

(22) Filed: Feb. 16, 2023

Related U.S. Application Data
(63) Continuation of application No. 16/322,212, filed on Jan. 31, 2019, now Pat. No. 11,753,638.
(60) Provisional application No. 62/374,499, filed on Aug. 12, 2016.
(60) Provisional application No. 62/461,529, filed on Feb. 21, 2017.

(51) Int. Cl. C12N 15/113; A61K 9/00; A61K 31/7088; A61K 47/54; A61K 47/55; A61P 1/00; A61P 1/16; A61P 5/00; A61P 9/00; A61P 11/00; A61P 13/12; C07H 21/00; C07H 21/02; C07H 21/04; C12N 15/11

(52) U.S. Cl. CPC C12N 15/113; A61K 9/0019; A61K 31/7088; A61K 47/54; A61K 47/543; A61K 47/549; A61K 47/551; A61K 47/554; A61P 1/00; A61P 1/16; A61P 5/00; A61P 9/00; A61P 11/00; A61P 13/12; C07H 21/00; C07H 21/02; C07H 21/04; C12N 15/111; C12N 2310/14; C12N 2310/312; C12N 2310/315; C12N 2310/346; C12N 2310/351; C12N 2310/3515; C12N 2320/32

(57) ABSTRACT

Provided herein are conjugated oligonucleotides that are characterized by efficient and specific tissue distribution.

### RELATED APPLICATIONS

This application is a continuation of U.S. patent application Ser. No. 16/322,212, filed Jan. 31, 2019, which is a 35 U.S.C. § 371 filing of International Patent Application No. PCT/US2017/046593, filed Aug. 11, 2017, which claims priority to U.S. Provisional Patent Application Serial Nos. 62/461,529, filed Feb. 21, 2017, and 62/374,499, filed Aug. 12, 2016, each of which is hereby incorporated herein by reference in its entirety for all purposes.

### STATEMENT OF FEDERALLY SPONSORED RESEARCH

This invention was made with government support under Grant Nos. GM108803 and TR000888 awarded by the National Institutes of Health. The Government has certain rights in this invention.

### SEQUENCE LISTING

The instant application contains a Sequence Listing which has been submitted electronically in XML format and is hereby incorporated by reference in its entirety. Said XML file, created on Feb. 16, 2023, is named 739944 UM9-215USCON_ST26.xml and is 14,038 bytes in size.

### TECHNICAL FIELD

This disclosure relates to novel hydrophobically-conjugated oligonucleotides. The oligonucleotide conjugates are designed to achieve unexpectedly high efficacy, uptake and tissue distribution.

### BACKGROUND

RNA interference represents a simple and effective tool for inhibiting the function of genes. The promise of RNA interference as a general therapeutic strategy, however, depends on the ability to deliver small RNAs to a wide range of tissues. Currently, small therapeutic RNAs can only be delivered effectively to liver. There remains a need for self-delivering siRNA that are characterized by efficient RISC entry, minimal immune response and off-target effects, efficient cellular uptake without formulation, and efficient and specific tissue distribution.

### SUMMARY

In one aspect, provided herein is a compound of formula (1):

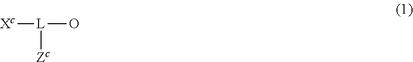

wherein:
O is a double-stranded nucleic acid comprising a first oligonucleotide and a second oligonucleotide, wherein:
   (1) the first oligonucleotide comprises at least 16 contiguous nucleotides, a 5′ end, a 3′ end and has complementarity to a target;
   (2) the second oligonucleotide comprises at least 15 contiguous nucleotides, a end, a 3′ end, and has homology with a target; and
   (3) a portion of the first oligonucleotide is complementary to a portion of the second oligonucleotide;
L is a divalent or trivalent linker;
   X^{c} is a hydrophobic moiety; and
   Z^{c} is a phosphodiester or phosphodiester derivative, or is absent.

In certain embodiments, L comprises an ethylene glycol chain, an alkyl chain, a peptide, RNA, DNA, a phosphodiester, a phosphorothioate, a phosphoramidate, an amide, a carbamate, or a combination thereof; and wherein L is attached to O via the second oligonucleotide.

In certain embodiments, Xc is selected from the group consisting of fatty acids, steroids, secosteroids, lipids, gangliosides and nucleoside analogs, endocannabinoids, and vitamins (e.g., a fatty acid selected from the group consisting of cholesterol, Lithocholic acid (LCA), Eicosapentaenoic acid (EPA), Docosahexaenoic acid (DHA) and Docosanoic acid (DCA), a vitamin selected from the group consisting of choline, vitamin A, vitamin E, and derivatives or metabolites thereof, or a vitamin selected from the group consisting of retinoic acid and alpha-tocopheryl succinate.

In certain embodiments, Xc has an affinity for low density lipoprotein and/or intermediate density lipoprotein or is a saturated or unsaturated moiety having fewer than three double bonds; has an affinity for high density lipoprotein (e.g., is a polyunsaturated moiety having three or more double bonds).

In certain embodiments, Z^{c} is selected from the group consisting of:

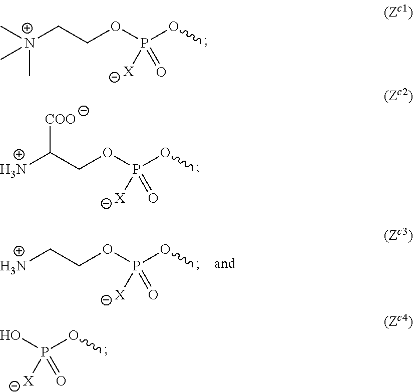

wherein X is O, S or BH₃. Zc may optionally be Zc1.

In certain embodiments, O comprises one or more chemically-modified nucleotides.

In certain embodiments, the first oligonucleotide comprises alternating 2′-methoxy-ribonucleotides and 2′-fluoro-ribonucleotides, the second oligonucleotide comprises alternating 2′-methoxy-ribonucleotides and 2′-fluoro-ribonucleotides and/or the nucleotides at positions 2 and 14 from the 5′ end of the second oligonucleotide are 2′-methoxy-ribonucleotides.

In certain embodiments, the nucleotides of the first oligonucleotide are connected via phosphodiester or phosphorothioate linkages, the nucleotides of the second oligonucleotide are connected via phosphodiester or phosphorothioate linkages, the nucleotides at positions 1 and 2 from the 3′ end of the second oligonucleotide are connected to adjacent nucleotides via phosphorothioate linkages, and/or the nucleotides at positions 1 and 2 from the 5′ end of the second oligonucleotide are connected to adjacent nucleotides via phosphorothioate linkages.

In certain embodiments, the first oligonucleotide comprises alternating 2′-methoxy-ribonucleotides and 2′-fluoro-ribonucleotides, wherein each nucleotide is a 2′-methoxy-ribonucleotide or a 2′-fluoro-ribonucleotide; and the nucleotides at positions 2 and 14 from the 5′ end of the first oligonucleotide are not 2′-methoxy-ribonucleotides; the second oligonucleotide comprises alternating 2′-methoxy-ribonucleotides and 2′-fluoro-ribonucleotides, wherein each nucleotide is a 2′-methoxy-ribonucleotide or a 2′-fluoro-ribonucleotide; and the nucleotides at positions 2 and 14 from the 5′ end of the second oligonucleotide are 2′-methoxy-ribonucleotides; the nucleotides of the first oligonucleotide are connected to adjacent nucleotides via phosphodiester or phosphorothioate linkages, wherein the nucleotides at positions 1-6 from the 3′ end, or positions 1-7 from the 3′ end are connected to adjacent nucleotides via phosphorothioate linkages; and the nucleotides of the second oligonucleotide are connected to adjacent nucleotides via phosphodiester or phosphorothioate linkages, wherein the nucleotides at positions 1 and 2 from the 3′ end are connected to adjacent nucleotides via phosphorothioate linkages.

In certain embodiments, the first oligonucleotide comprises a moiety X at the 5′ end, wherein X is selected from the group consisting of:

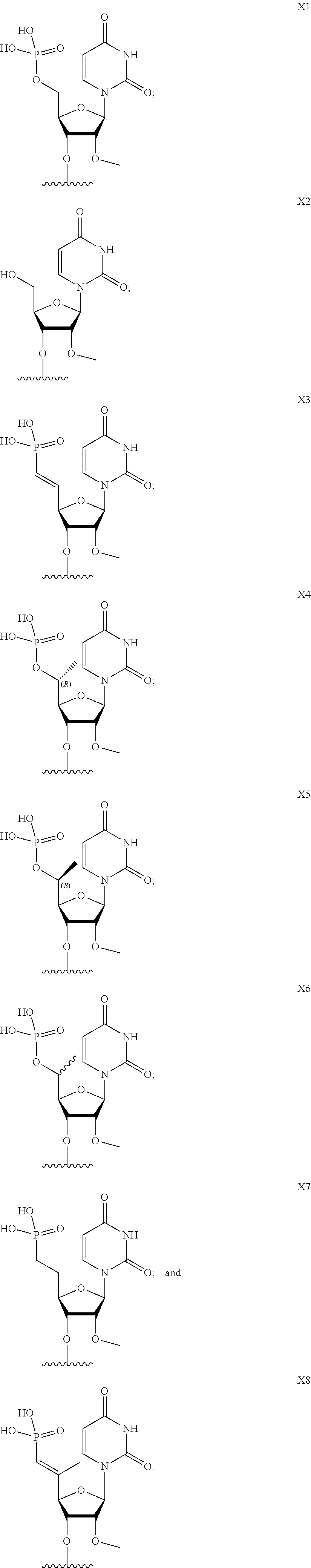

In certain embodiments, X is X3.

In certain embodiments, the first oligonucleotide has the structure of formula (Ia):

X(—K—B—K-A)ⱼ(—S—B—S-A)ᵣ(—S—B)ₜ—OR (Ia)
wherein:
   X is selected from the group consisting of:

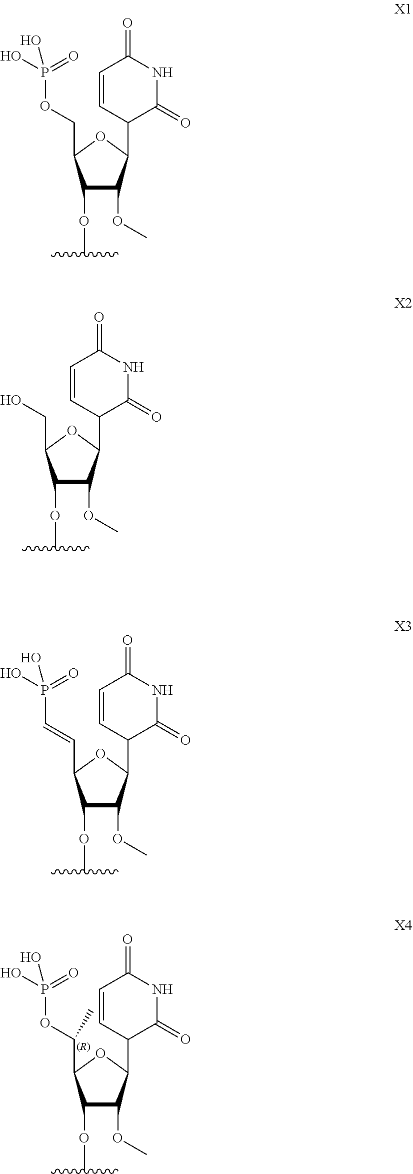

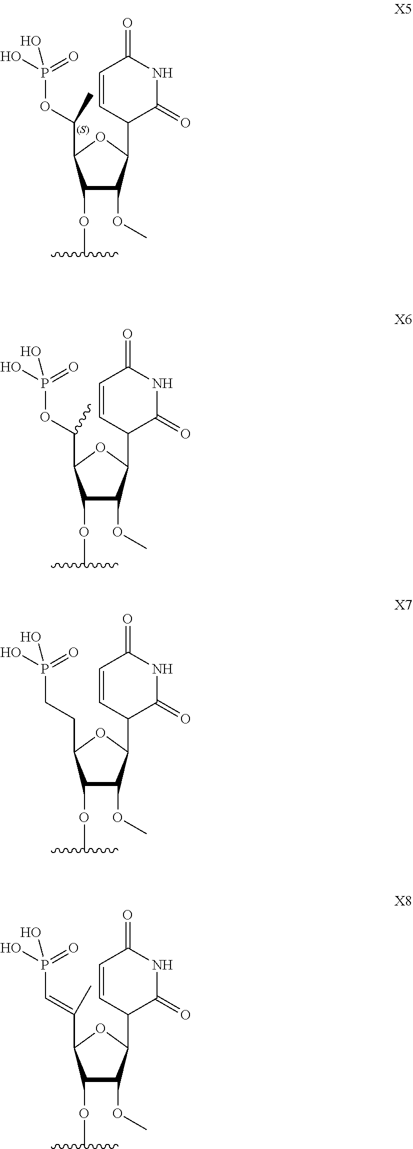

A, for each occurrence, independently is a 2′-methoxy-ribonucleotide;
B, for each occurrence, independently is a 2′-fluoro-ribonucleotide;
K, for each occurrence independently is a phosphodiester or phosphorothioate linker;
S is a phosphorothioate linker;
R is hydrogen, phosphate, vinylphosphonate, or a capping group;
j is 4, 5, 6 or 7;
r is 2 or 3; and
t is 0 or 1.

In certain embodiments, the first oligonucleotide has the structure of formula (IIa):

C-L-B(—S-A-S—B)_{m′}(—P-A-P—B)_{n′}(—P-A-S—B)_{q′}(—S-A)_{r′}(—S—B)_{t′}—OR′ (IIa)
wherein:
   C-L is:

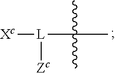

A, for each occurrence, independently is a 2′-methoxy-ribonucleotide;
B, for each occurrence, independently is a 2′-fluoro-ribonucleotide;
S is a phosphorothioate linker;
P is a phosphodiester linker;
R′ is hydrogen, phosphate, vinylphosphonate, or a capping group;
m′ is 0 or 1;
n′ is 4, 5 or 6;
q′ is 0 or 1;
r′ is 0 or 1; and
t′ is 0 or 1.

In certain embodiments, the first oligonucleotide has 3-7 more nucleotides than the second oligonucleotide, the sequences of the first and second oligonucleotides comprise sequences selected from the tables of FIG. 7, Xc is DHA, Zc is not Zc1, and/or Zc is Zc1 and Xc is not DHA.

In certain embodiments, the first oligonucleotide has perfect complementarity to the target, the second oligonucleotide has complete homology with the target, and/or the target is mammalian or viral mRNA (e.g., an intronic region of said mRNA).

In one aspect, a method for delivering any of the compounds described herein to an organ or tissue in a patient, comprising administering said compound to the patient, wherein the compound has a selective affinity for a serum lipoprotein is provided.

In certain embodiments, the organ is the kidney, the brain or the epidermis, and the compound has a selective affinity for high density lipoprotein versus low density lipoprotein and/or high density lipoprotein. The compound may be to the patient intravenously or subcutaneously. Xc may be a polyunsaturated moiety having three or more double bonds.

In certain embodiments, a method for treating a disease or disorder of the kidneys, brain, epidermis or liver, in a patient in need of such treatment, comprising administering to the patient a compound described herein is provided.

In certain embodiments, the disease or disorder is selected from the group consisting of glomerulonephritis, glomerulosclerosis, nephrolithiasis, Lightwood-Albright syndrome, polycystic kidney disease, acute renal failure, acute renal injury, chronic kidney disease, kidney fibrosis, diabetic nephropathy, Fabry disease, Fanconi syndrome, focal segmental glomerulosclerosis, Goodpasture syndrome, Liddle syndrome, nutcracker syndrome, peritoneal-renal syndrome, and renal cell cancer.

In certain embodiments, the disease or disorder is selected from the group consisting of: Alzheimer's disease, amyotrophic lateral sclerosis, aneurysm, attention deficit disorder, attention deficit hyperactivity disorder, autism spectrum disorder, brain cancer, concussion, coma, cerebral palsy, dementia, dyslexia, epilepsy, encephalitis, Friedreich's ataxia, Huntington's disease, migraine, multiple sclerosis, narcolepsy, Parkinson's disease, stroke, and traumatic brain injury.

In certain embodiments, the disease or disorder is selected from the group consisting of: ichthyosis, ectodermal dysplasia, psoriasis, eczema, Darier's disease, porokeratosis, acne, vitiligo, and skin cancer.

In certain embodiments, the disease or disorder is selected from the group consisting of: liver disease, cirrhosis, fatty liver, liver cancer, hemochromatosis, toxic hepatitis, viral hepatitis, Gibert's syndrome, galactosemia, cystic disease of the liver, and Alagille syndrome.

In another aspect, a method for delivering a compound described herein to an organ or tissue in a patient, comprising administering to the patient said compound, wherein the organ or tissue is selected from the group consisting of thymus, bladder, intestine, skin, bone marrow, placenta, adipose tissue, muscle, spleen, pancreas, lung, fallopian tube, adrenal gland, heart, liver and kidney, is provided.

In certain embodiments, X^{c} is selected from the group consisting of DHA g1, DHA g2, DCA g1, DCA g2, EPA g1, EPA g2, cholesterol g1, cholesterol C7 g1, cholesterol g2, LA g1, LA g2, RA g1, RA g2, TOCO g1 and TOCO g2.

In certain embodiments, the organ is the thymus and X^{c} is selected from the group consisting of RA g1, RA g2, DHA g1, DHA g2 and DCA g2.

In certain embodiments, the organ is the bladder and X^{c} is selected from the group consisting of DHA g1, DHA g2, EPA g1, LA g1 and LA g2.

In certain embodiments, the organ is the intestine and X^{c} is selected from the group consisting of EPA g1, EPA g2, RA g1, DHA g1, DHA g2 and LA g2, (e.g., selected from RA g1, EPA g1 and EPA g2).

In certain embodiments, the organ is the skin and X^{c} is selected from the group consisting of RA g1, RA g2, EPA g1, EPA g2, DHA g1, DHA g2, DCA g1, DCA g2, LA g1, LA g2, cholesterol g1, cholesterol C7 g1 and cholesterol g2.

In certain embodiments, the tissue is bone marrow and X^{c} is selected from the group consisting of RA g1, RA g2, TOCO g1, DHA g1, DHA g2, EPA g1, EPA g2, DCA g1, DCA g2, LA g1, LA g2, cholesterol C7 g1, cholesterol g2 and choline.

In certain embodiments, the organ is the placenta and X^{c} is selected from the group consisting of cholesterol g1, DCA g1, DCA g2, DHA g1, DHA g2 (e.g., wherein Xc is DCA g1 or DCA g2).

In certain embodiments, the tissue is adipose tissue and X^{c} is selected from the group consisting of RA g1, DHA g1, DHA g2, EPA g1, DCA g1, DCA g2, LA g1 and LA g2.

In certain embodiments, the tissue is muscle tissue and X^{c} is selected from the group consisting of TOCO g1, DHA g2, EPA g1, EPA g2 and DCA g2.

In certain embodiments, the organ is spleen and X^{c} is selected from the group consisting of RA g1, RA g2, TOCO g1, TOCO g2, DHA g1, DHA g2, EPA g1, EPA g2, DCA g1, DCA g2, LA g1, LA g2, cholesterol g1, cholesterol C7 g1 and cholesterol g2 (e.g., selected from cholesterol g1, cholesterol g2, DCA g1 and DCA g2).

In certain embodiments, the organ or tissue is pancreas and Xc is selected from the group consisting of RA g2, DHA g2, EPA g1, DCA g2 and LA g1.

In certain embodiments, the organ is lung and X^{c} is selected from the group consisting of RA g1, RA g2, EPA g1, EPA g2, DCA g1 and DCA g2 (e.g., selected from DCA g1, DCA g2, EPA g1 and EPA g2).

In certain embodiments, the organ is the fallopian tube and X^{c} is selected from the group consisting of RA g1, TOCO g1, EPA g2, DCA g2, LA g2 and cholesterol g2.

In certain embodiments, the organ is the adrenal gland and X^{c} is selected from the group consisting of RA g1, RA g2, DCA g1, DCA g2, LA g1, LA g2, cholesterol g1, cholesterol C7 g1 and cholesterol g2 (e.g., selected from RA g1, DCA g1, DCA g2, cholesterol g1 and cholesterol g2).

In certain embodiments, the organ is the heart and X^{c} is selected from the group consisting of DCA g1, DCA g2, EPA g1, EPA g2, cholesterol g1 and cholesterol g2.

In certain embodiments, the organ is the kidney and X^{c} is selected from the group g2, LA g1, LA g2, cholesterol g1 and choline (e.g., selected from RA g1, EPA g1, EPA g2, DCA g2 and cholesterol g1).

In certain embodiments, the organ is the liver and X^{c} is selected from the group consisting of RA g1, RA g2, TOCO g1, TOCO g2, DHA g1, DHA g2, EPA g1, EPA g2, DCA g1, DCA g2, LA g1, cholesterol g1, cholesterol C7 g1 and cholesterol g2 (e.g., selected from RA g1, DCA g1, DCA g2, EPA g1, EPA g2, cholesterol g1 and cholesterol g2).

In one aspect, a method for treating a disease or disorder of adipose tissue, fallopian tube, adrenal gland, spleen, pancreas, or adipose tissue, in a patient in need of such treatment, comprising administering to the patient a compound described herein, is provided.

In certain embodiments, the disease or disorder is selected from the group consisting of: obesity, diabetes, insulin resistance, lipodystrophies, Dercum's disease, adipose tissue neoplasm, general adipose tissue inflammation, cardiovascular disease, hypertension and stroke, hypercholesterolemia, hypertriglyceridemia, arthritis, asthma and cancer.

In certain embodiments, the disease or disorder is selected from the group consisting of: salpingitis, endosalpingiosis, tubal torsion, paratubal cyst, endometriosis, fallopian tube cancers, infertility, fallopian tube obstruction, and adhesions.

In certain embodiments, the disease or disorder is selected from the group consisting of: Addison's disease, adrenal tumors, adrenal insufficiency, adrenal hyperplasia, primary aldosteronism, hyperaldosteronism, hypoaldosteronism, adrenal crisis, Cushing's disease, adrenocortical hyperfunction, adrenoleukodystrophy, adrenal fatigue, and adrenal incidentaloma.

In certain embodiments, the disease or disorder is selected from the group consisting of: splenomegaly, splenic disease, Gaucher's disease, asplenia, splenic infarction, spherocytosis, wandering spleen, splenic tumors, infectious mononucleosis, splenic injury, hyaloserositis, and anemias.

In certain embodiments, the disease or disorder is selected from the group consisting of: pancreatitis, pancreatic cancer, cystic fibrosis, pseudocyst, exocrine pancreatic insufficiency, diabetes, gastrointestinal diseases, pancreas divisum, steatorrhea, and sphincter of Oddi dysfunction.

In certain embodiments, the organ or tissue is adipose tissue, Xc is choline and Zc is absent.

In certain embodiments, the organ or tissue is adipose tissue, and wherein Xc is choline and Zc is absent.

In certain embodiments, the organ or tissue is the adrenal gland, and wherein Xc is LCA and Zc is Zc1.

In certain embodiments, the organ or tissue is fallopian tube, and wherein Xc is EPA and Zc is Zc1.

In certain embodiments, the organ or tissue is muscle, and wherein Xc is LCA and Zc is absent.

In certain embodiments, the organ or tissue is the pancreas, and wherein Xc is EPA and Zc is Zc1.

In certain embodiments, the organ or tissue is the heart.

In another aspect, a compound of formula (I):

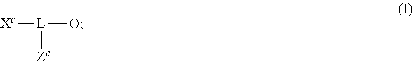

wherein:
   O is an oligonucleotide;
   L is a divalent or trivalent linker selected from an ethylene glycol chain, an alkyl
   chain, a peptide, RNA, DNA, a phosphodiester, a phosphorothioate, a
   phosphoramidate, an amide, a carbamate, or a combination thereof;
   X^{c} is a hydrophobic moiety; and
   Z^{c} is a phosphodiester or phosphodiester derivative, or is absent, is provided.

In another aspect, a compound of formula (I):

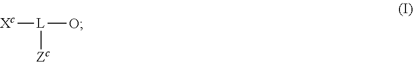

wherein:
   O is an oligonucleotide;
   L is a divalent or trivalent linker a divalent or trivalent linker selected from an ethylene glycol chain, an alkyl chain, a peptide, RNA, DNA, a phosphodiester, a phosphorothioate, a phosphoramidate, an amide, a carbamate, or a combination thereof;
   X^{c} is a hydrophobic moiety selected from fatty acids, steroids, secosteroids, lipids, gangliosides and nucleoside analogs, or endocannabinoids; and
   Z^{c} is absent, or is a phosphodiester or phosphodiester derivative selected from

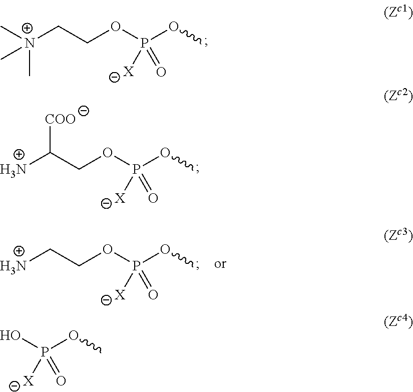

wherein X is O, S or BH₃, is provided.

In another aspect, a compound of formula (I):

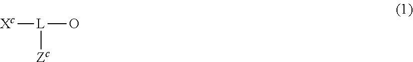

wherein:
   O is an oligonucleotide;
   L is a divalent or trivalent linker selected from an ethylene glycol chain, an alkyl chain, a peptide, RNA, DNA, a phosphodiester, a phosphorothioate, a phosphoramidate, an amide, a carbamate, or a combination thereof;
   X^{c} is a hydrophobic moiety selected from fatty acids, steroids, vitamins, secosteroids, lipids, gangliosides and nucleoside analogs, or endocannabinoids; and
   Z^{c} is absent or is a phosphodiester or phosphodiester derivative selected from

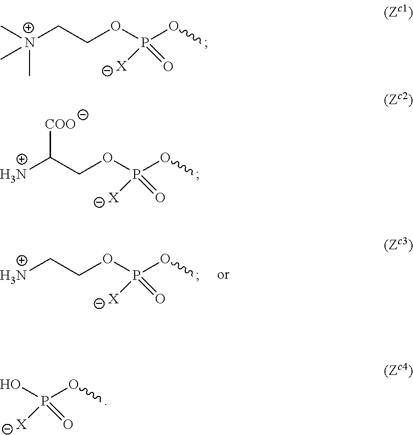

wherein X is O, S or BH₃, is provided.

In another aspect, a compound of formula (I):

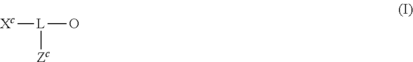

wherein:
   O is an oligonucleotide;
   L is a divalent or trivalent linker selected from:

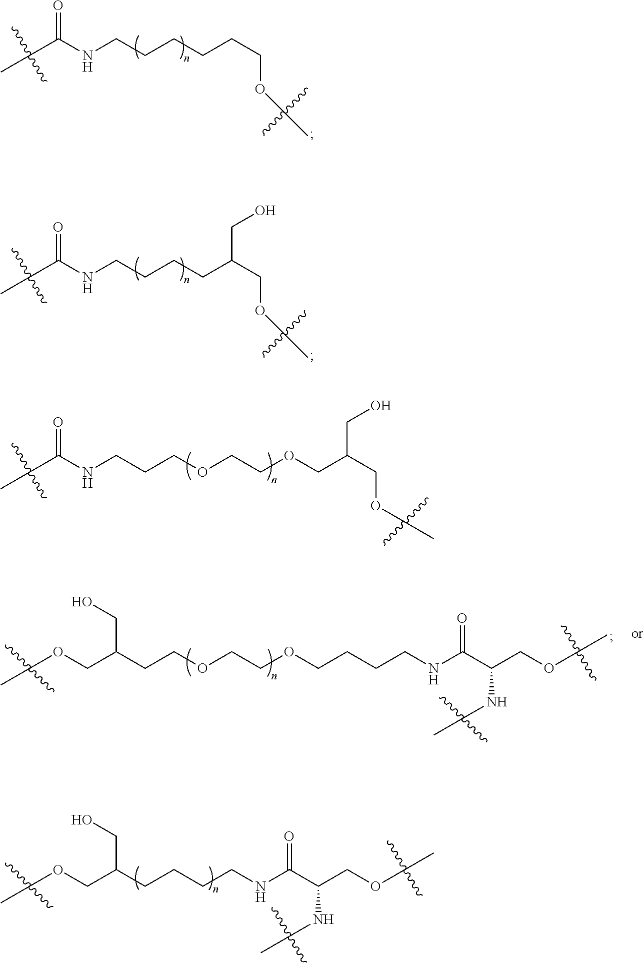

wherein n is 1, 2, 3, 4, or 5;
   X^{c} is DHA, DCA, EPA, cholesterol, Lithocholic acid, Retinoic acid or α-tocopheryl succinate; and
   Z^{c} is absent, or is a phosphodiester or phosphodiester derivative selected from

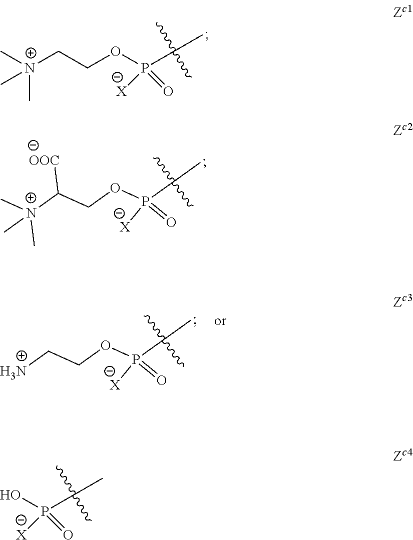

wherein X is O, S or BH₃, is provided.

In another aspect, a compound of formula (I):

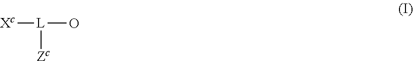

wherein:
   O is an oligonucleotide;
   L is a linker selected from:

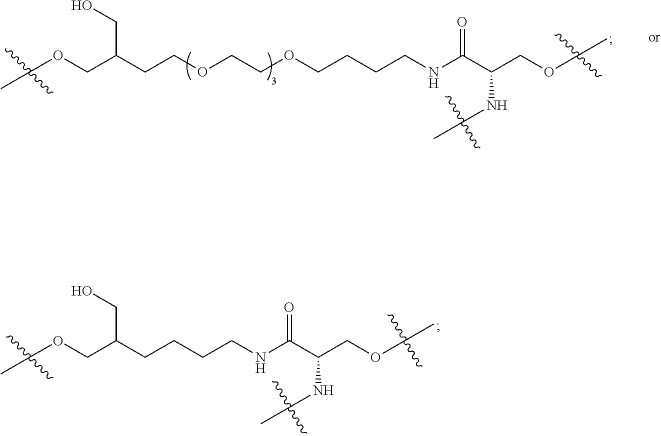

X^{c} is a DHA, DCA, EPA, cholesterol, Lithocholic acid, Retinoic acid or α-tocopheryl succinate; and
Z^{c} is a phosphodiester or phosphodiester derivative selected from

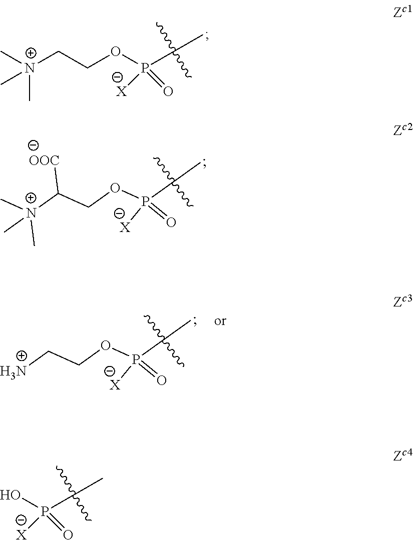

wherein X is O, S or BH₃, is provided.

In another aspect, a

compound comprising a structure selected from:

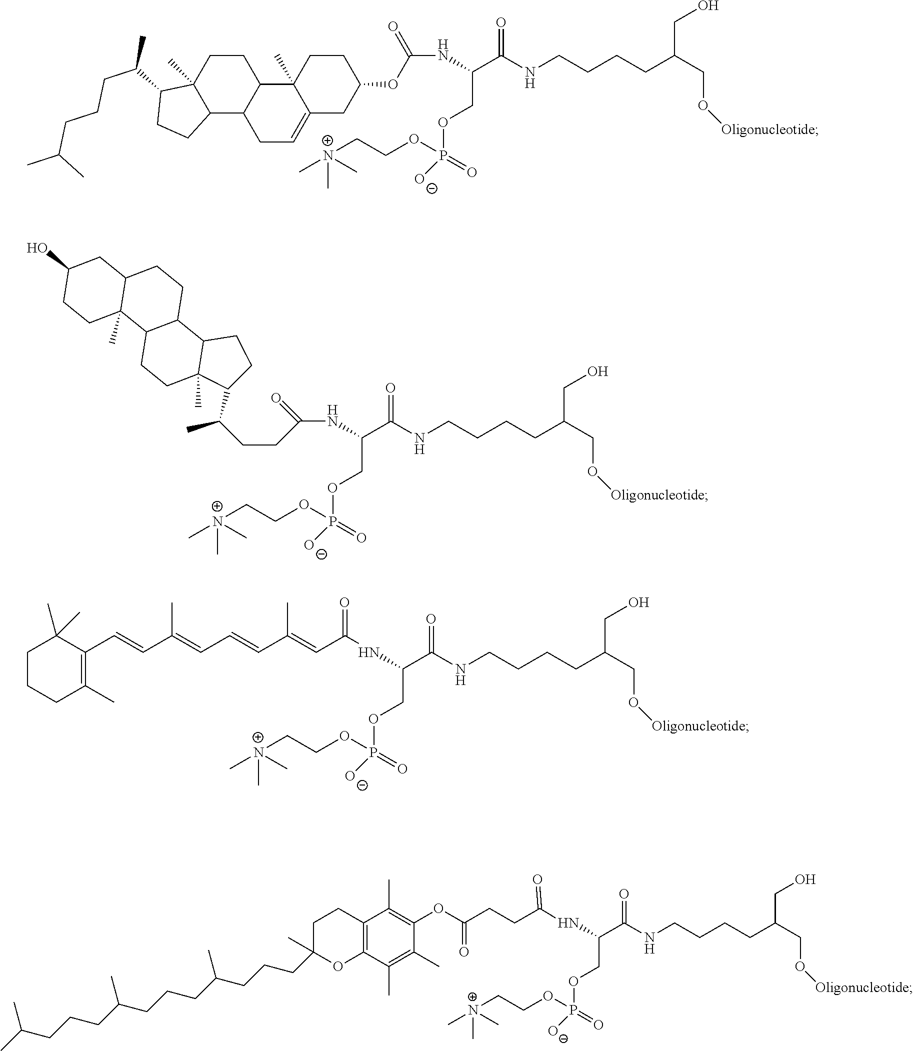

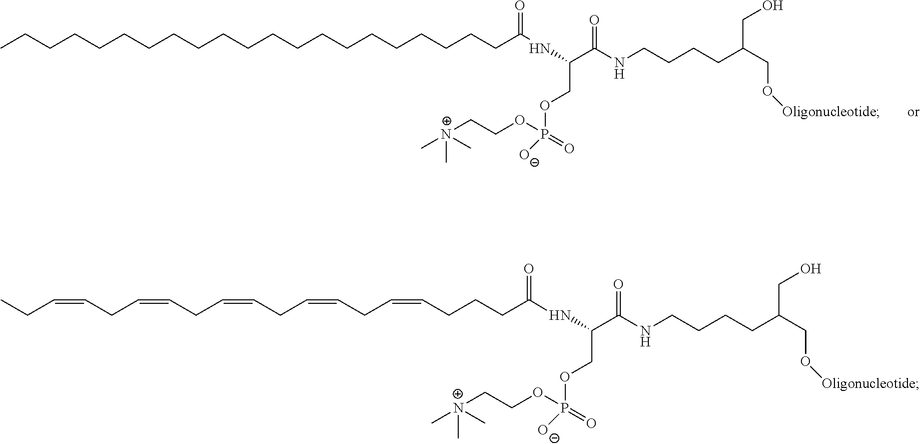

is provided.

In another aspect, a method for delivering the compound according to a compound described herein to an organ or tissue in a subject, comprising administering said compound to the subject, wherein Xc is DHA g1 or DHA g2, and wherein the organ or tissue is selected from the group consisting of thymus, bladder, intestine, skin, bone marrow, placenta, adipose, muscle, spleen, pancreas, liver and kidney, is provided.

In certain embodiments, the compound is delivered subcutaneously or intravenously.

In another aspect, a method for delivering the compound according to a compound described herein to an organ or tissue in a subject, comprising administering said compound to the subject, wherein Xc is DCA g1 or DCA g2, and wherein the organ or tissue is selected from the group consisting of thymus, skin, bone marrow, placenta, adipose, muscle, spleen, pancreas, lung, fallopian tube, adrenal gland, heart, liver and kidney, is provided.

In certain embodiments, the compound is delivered subcutaneously or intravenously.

In another aspect, a method for delivering the compound according to a compound described herein to an organ or tissue in a subject, comprising administering said compound to the subject, wherein Xc is EPA g1 or EPA g2, and wherein the organ or tissue is selected from the group consisting of thymus, bladder, intestine, skin, bone marrow, adipose, muscle, spleen, pancreas, lung, fallopian tube, heart, liver and kidney, is provided.

In certain embodiments, the compound is delivered subcutaneously or intravenously.

In another aspect, a method for delivering the compound according to a compound described herein to an organ or tissue in a subject, comprising administering said compound to the subject, wherein Xc is cholesterol g1, cholesterol C7 g1 or cholesterol g2, and wherein the organ or tissue is selected from the group consisting of skin, bone marrow, placenta, spleen, fallopian tube, adrenal gland, heart, liver and kidney, is provided.

In certain embodiments, the compound is delivered subcutaneously or intravenously.

In another aspect, a method for delivering the compound according to a compound described herein to an organ or tissue in a subject, comprising administering said compound to the subject, wherein Xc is LA g1 or LA g2, and wherein the organ or tissue is selected from the group consisting of bladder, intestine, skin, bone marrow, adipose, spleen, pancreas, fallopian tube, adrenal gland, liver and kidney, is provided.

In certain embodiments, the compound is delivered subcutaneously or intravenously.

In another aspect, a method for delivering the compound according to a compound described herein to an organ or tissue in a subject, comprising administering said compound to the subject, wherein Xc is RA g1 or RA g2, and wherein the organ or tissue is selected from the group consisting of thymus, intestine, skin, bone marrow, adipose, spleen, pancreas, lung, fallopian tube, adrenal gland, liver and kidney, is provided.

In certain embodiments, the compound is delivered subcutaneously or intravenously.

In another aspect, a method for delivering the compound according to a compound described herein to an organ or tissue in a subject, comprising administering said compound to the subject, wherein Xc is TOCO g1 or TOCO g2, and wherein the organ or tissue is selected from the group consisting of bone marrow, muscle, spleen, fallopian tube, liver and kidney, is provided.

In certain embodiments, the compound is delivered subcutaneously or intravenously.

In another aspect, a method for treating a disease or disorder of an organ or tissue selected from the group consisting of thymus, bladder, intestine, skin, bone marrow, adipose tissue, muscle, spleen, pancreas, lung, fallopian tube, adrenal gland, heart, liver and kidney in a patient in need of such treatment, comprising administering to the patient a compound described herein, is provided.

In certain embodiments, X^{c} is selected from the group consisting of DHA g1, DHA g2, DCA g1, DCA g2, EPA g1, EPA g2, cholesterol g1, cholesterol C7 g1, cholesterol g2, LA g1, LA g2, RA g1, RA g2, TOCO g1 and TOCO g2.

In certain embodiments, the organ is the thymus and X^{c} is selected from the group consisting of RA g1, RA g2, DHA g1, DHA g2 and DCA g2.

In certain embodiments, the organ is the bladder and X^{c} is selected from the group consisting of DHA g1, DHA g2, EPA g1, LA g1 and LA g2.

In certain embodiments, the organ is the intestine and Xc is selected from the group consisting of EPA g1, EPA g2, RA g1, DHA g1, DHA g2 and LA g2 (e.g., selected from RA g1, EPA g1 and EPA g2).

In certain embodiments, the organ is the skin and X^{c} is selected from the group consisting of RA g1, RA g2, EPA g1, EPA g2, DHA g1, DHA g2, DCA g1, DCA g2, LA g1, LA g2, cholesterol g1, cholesterol C7 g1 and cholesterol g2.

In certain embodiments, the tissue is bone marrow tissue and X^{c} is selected from the group consisting of RA g1, RA g2, TOCO g1, DHA g1, DHA g2, EPA g1, EPA g2, DCA g1, DCA g2, LA g1, LA g2, cholesterol C7 g1, cholesterol g2 and choline.

In certain embodiments, the organ is the placenta and X^{c} is selected from the group consisting of cholesterol g1, DCA g1, DCA g2, DHA g1 and DHA g2 (e.g., X^{c} is DCA g1 or DCA g2).

In certain embodiments, the tissue is adipose tissue and X^{c} is selected from the group consisting of RA g1, DHA g1, DHA g2, EPA g1, DCA g1, DCA g2, LA g1 and LA g2.

In certain embodiments, the organ or tissue is muscle and X^{c} is selected from the group consisting of TOCO g1, DHA g2, EPA g1, EPA g2 and DCA g2.

In certain embodiments, the organ is spleen and X^{c} is selected from the group consisting of RA g1, RA g2, TOCO g1, TOCO g2, DHA g1, DHA g2, EPA g1, EPA g2, DCA g1, DCA g2, LA g1, LA g2, cholesterol g1, cholesterol C7 g1 and cholesterol g2 (e.g., X^{c} is selected from cholesterol g1, cholesterol g2, DCA g1 and DCA g2).

In certain embodiments, the organ is the pancreas and X^{c} is selected from the group consisting of RA g2, DHA g2, EPA g1, DCA g2 and LA g1.

In certain embodiments, the organ is the lung and Xc is selected from the group consisting of RA g1, RA g2, EPA g1, EPA g2, DCA g1 and DCA g2 (e.g., selected from DCA g1, DCA g2, EPA g1 and EPA g2).

In certain embodiments, the organ is the fallopian tube and X^{c} is selected from the group consisting of RA g1, TOCO g1, EPA g2, DCA g2, LA g2 and cholesterol g2.

In certain embodiments, the organ is the adrenal gland and X^{c} is selected from the group consisting of RA g1, RA g2, DCA g1, DCA g2, LA g1, LA g2, cholesterol g1, cholesterol C7 g1 and cholesterol g2 (e.g., X^{c} is selected from RA g1, DCA g1, DCA g2, cholesterol g1 and cholesterol g2).

In certain embodiments, the organ is the heart and X^{c} is selected from the group consisting of DCA g1, DCA g2, EPA g1, EPA g2, cholesterol g1 and cholesterol g2.

In certain embodiments, the organ is the kidney and Xc is selected from the group g2, LA g1, LA g2, cholesterol g1 and choline (e.g., X^{c} is selected from RA g1, EPA g1, EPA g2, DCA g2 and cholesterol g1).

In certain embodiments, the organ or tissue is the liver and Xc is selected from the group consisting of RA g1, RA g2, TOCO g1, TOCO g2, DHA g1, DHA g2, EPA g1, EPA g2, DCA g1, DCA g2, LA g1, cholesterol g1, cholesterol C7 g1 and cholesterol g2 (e.g., selected from RA g1, DCA g1, DCA g2, EPA g1, EPA g2, cholesterol g1 and cholesterol g2).

In certain embodiments, the organ is the thymus and the disease or disorder is selected from the group consisting of myasthenia gravis, pure red cell aplasia, hypogammaglobulinemia and cancer.

In certain embodiments, the organ is the bladder and the disease or disorder is selected from the group consisting of flaccid neurogenic bladder, spastic bladder, mixed type flaccid and spastic bladder, overflow incontinence, overflow dribbling, urinary tract infections, urinary calculi, cystitis, interstitial cystitis, bladder rupture, bladder obstruction, paruresis, cystocele, bladder fistula, bladder stones, bladder exstrophy, bladder diverticulum and cancer.

In certain embodiments, the organ is the intestine and the disease or disorder is selected from the group consisting of celiac disease, Crohn's disease, irritable bowel syndrome, ulcer (e.g., peptic ulcer), intestinal dysmobility, intestinal pseudo-obstruction, short bowel syndrome, intestinal malrotation, Meckel's diverticulum, superior mesenteric artery syndrome, necrotizing enterocolitis, duodenal artesia, enteritis, small bowel bacterial overgrowth, Yersinia enterocolitica infection, Yersinia pseudotuberculosis infection and cancer.

In certain embodiments, the organ is the skin and the disease or disorder is selected from the group consisting of ichthyosis, ectodermal dysplasia, psoriasis, eczema, Darier's disease, porokeratosis, acne, vitiligo and skin cancer.

In certain embodiments, the tissue is bone tissue and the disease or disorder is selected from the group consisting of leukemia, lymphoma, aplastic anemia, a myeloproliferative disorder and a myelodysplastic syndrome.

In certain embodiments, the organ is the placenta and the disease or disorder is selected from the group consisting of abruptio placentae, placenta accretia, placenta increta, placenta percreta, chorioamnionitis, intervillitis, TORCH infections (e.g., CMV placentitis), chronic deciduitis, circumvallate placenta, placental villous immaturity, placenta previa, vasa previa, fetal thrombic vasculopathy, hypertrophic decidual vasculopathy, chorangiosis, chorangioma, placental infarction, hydatidiform mole, choriocarcinoma and placental cancer.

In certain embodiments, the tissue is adipose tissue and the disease or disorder is selected from the group consisting of obesity, diabetes, insulin resistance, a lipodystrophy, Dercum's disease, adipose tissue neoplasm, general adipose tissue inflammation, cardiovascular disease, hypertension, stroke, hypercholesterolemia, hypertriglyceridemia, arthritis, asthma and cancer.

In certain embodiments, the tissue is muscle tissue and the disease or disorder is selected from the group consisting of myositis, myotonia congenita, paramyotonia congenita, periodic paralyses, central core disease/malignant hyperthermia susceptibility, nemaline myopathy, centronuclear myopathies, sarcopenia, mitochondrial myopathies, hypotonia, muscular dystrophy, dermatomyositis, cerebral palsy, compartment syndrome, myasthenia gravis, amyotrophic lateral sclerosis, rhabdomyolysis, polymyositis, fibromyalgia, myofascial pain syndrome, muscle cramp, muscle sprain, muscle strain and tendonitis.

In certain embodiments, the organ is the spleen and the disease or disorder is selected from the group consisting of splenomegaly, splenic disease, Gaucher's disease, asplenia, splenic infarction, spherocytosis, wandering spleen, splenic tumor, infectious mononucleosis, hyaloserositis and anemia.

In certain embodiments, the organ is the pancreas and the disease or disorder is selected from the group consisting of pancreatitis, pancreatic cancer, cystic fibrosis, pseudocyst, exocrine pancreatic insufficiency, diabetes, gastrointestinal diseases, pancreas divisum, steatorrhea and sphincter of Oddi dysfunction.

In certain embodiments, the organ is the lung and the disease or disorder is selected from the group consisting of chronic obstructive pulmonary disease, asthma, chronic bronchitis, acute bronchitis, emphysema, cystic fibrosis, pneumonia, tuberculosis, pulmonary edema, lung cancer, acute respiratory distress syndrome, pneumoconiosis, interstitial lung disease, pulmonary embolism, pulmonary hypertension, pleural effusion, pneumothorax, mesothelioma and obesity hypoventilation syndrome.

In certain embodiments, the organ is the fallopian tube and the disease or disorder is selected from the group consisting of salpingitis, endosalpingiosis, tubal torsion, paratubal cyst, endometriosis, fallopian tube cancer, infertility, fallopian tube obstruction and one or more adhesions.

In certain embodiments, the organ is the adrenal gland and the disease or disorder is selected from the group consisting of Addison's disease, adrenal tumors, adrenal insufficiency, adrenal hyperplasia, primary aldosteronism, hyperaldosteronism, hypoaldosteronism, adrenal crisis, Cushing's disease, adrenocortical hyperfunction, adrenoleukodystrophy, adrenal fatigue and adrenal incidentaloma.

In certain embodiments, the organ is the brain and the disease or disorder is selected from the group consisting of Alzheimer's disease, amyotrophic lateral sclerosis, aneurysm, attention deficit disorder, attention deficit hyperactivity disorder, autism spectrum disorder, brain cancer, concussion, coma, cerebral palsy, dementia, dyslexia, epilepsy, encephalitis, Friedreich's ataxia, Huntington's disease, migraine, multiple sclerosis, narcolepsy, Parkinson's disease, stroke, and traumatic brain injury.

In certain embodiments, the organ is the heart and the disease or disorder is selected from the group consisting of atherosclerosis, coronary artery disease, myocarditis, endocarditis, pericarditis, rheumatic heart disease, hypertensive heart disease, aneurysm, angina, myocardial infarction, cardiomyopathy, valvular heart disease, congential heart disease, heart failure, arrhythmia, cardiac arrest, congestive heart failure, atrial fibrillation, Brugada syndrome, tachycardia, catecholaminergic polymorphic ventricular tachycardia, long QT syndrome, progressive cardiac conduction defect, stroke, peripheral artery disease, thromboembolism, high blood pressure, heart murmur, Kawasaki disease, DiGeorge syndrome, pre-eclampsia and cardiac tumor.

In certain embodiments, the organ is the kidney and the disease or disorder is selected from the group consisting of glomerulonephritis, glomerulosclerosis, nephrolithiasis, Lightwood-Albright syndrome, polycystic kidney disease, acute renal failure, acute renal injury, chronic kidney disease, kidney fibrosis, diabetic nephropathy, Fabry disease, Fanconi syndrome, focal segmental glomerulosclerosis, Goodpasture syndrome, Liddle syndrome, nutcracker syndrome, peritoneal-renal syndrome, and renal cell cancer.

In certain embodiments, the organ is the liver and the disease or disorder is selected from the group consisting of liver disease, cirrhosis, fatty liver, liver cancer, hemochromatosis, toxic hepatitis, viral hepatitis, Gibert's syndrome, galactosemia, cystic disease of the liver and Alagille syndrome.

### BRIEF DESCRIPTION OF THE DRAWINGS

FIG. 1A shows a synthetic approach for cortisol-conjugated oligonucleotides.

FIG. 1B shows a synthetic approach for calciferol-conjugated oligonucleotides.

FIG. 1C shows a synthetic approach for DHA-conjugated oligonucleotides.

FIG. 1D shows a synthetic approach for preparation of an alkynylated-oligonucleotide for click conjugation.

FIG. 1E shows a synthetic approach for GM1-conjugated oligonucleotides.

FIG. 1F shows a synthetic approach for lysophosphatidylcholine esterified DHA-hsiRNA conjugate (referred to as DHAPCL-hsiRNA, PC-DHA-hsiRNA, g2DHA-hsiRNA, or DHA-G2-hsiRNA).

FIG. 1G shows a synthetic approach for an hsiRNA-Calciferol oligonucleotide (SEQ ID NO: 1).

FIG. 1H shows an alternative synthetic approach for an hsiRNA-Calciferol oligonucleotide.

FIG. 1I shows a representative analytical HPLC trace of a synthesized hsiRNA conjugate, and its stability at room temperature immediately after purification, after 24 hours at room temperature, and after 48 hours at room temperature; sFLT-g2DHA-Cy3-P2 is shown.

FIG. 1J shows a representative analytical HPLC trace and an ESI-MS spectra of a synthesized hsiRNA conjugate; lysophosphatidylcholine esterified DHA-hsiRNA conjugate is shown.

FIG. 1K shows a representative analytical HPLC trace of an hsiRNA conjugate prepared according to the synthetic approach of FIG. 1I; hsiRNA-Calciferol shown.

FIG. 1L shows a representative semi-prep reverse-phase-HPLC trace of a synthesized hsiRNA conjugate; Cy3-labeled sFLT-DHA conjugate (crude reaction mixture) shown.

FIG. 1M shows a representative analytical reverse-phase-HPLC following purification of a synthesized hsiRNA conjugate as in FIG. 1L; Cy3-labeled sFLT-DHA conjugate (pure product) shown.

FIG. 1N shows a representative LC-MS profile following purification of a synthesized hsiRNA conjugate as in FIG. 1L; Cy3-labeled sFLT-DHA conjugate (pure product) shown.

FIG. 2A depicts exemplary hydrophobic moieties.

FIG. 2B shows the differences in hydrophobicity profiles of synthesized siRNA conjugates as observed by reverse-phase HPLC (C8).

FIG. 2C shows an exemplary LC-MS analysis of a synthesized hsiRNA conjugate; DHA-hsiRNA shown.

FIG. 3A shows a biodistribution study protocol.

FIG. 3B shows that the in vivo brain distribution of FMS-hsiRNA is defined by conjugation modality.

FIG. 4 shows accumulation in various tissues upon systemic administration of hsiRNA-conjugates. hsiRNA-conjugate structures and modifications are found in FIG. 5A-F. All compounds have the sequence of PPIB, as shown in FIG. 7.

FIG. 5A-F show chemical structures of conjugated hsiRNAs. (A) Docosanoic (DCA)-conjugated hsiRNA. (B) Docosahexaenoic acid (DHA)-conjugated hsiRNA, 22:6 (n−3). (C) Phosphatidylcholine-DHA-conjugated hsiRNA (g2DHA-hsiRNA or DHAPCL-hsiRNA) (D) Eicosapentanoic acid (EPA)-conjugated hsiRNA, 20:5(n−3). (E) Cholesterol (Chop-conjugated hsiRNA. (F) Cholesterol (Chop-conjugated hsiRNA. hsiRNA conjugates represented to scale using PyMOL.

FIG. 6A-B show PNA (Peptide Nucleic Acid) based assay for detection of hsiRNA guide strand in mouse tissues. (A) Tissues were lysed, debris separated by precipitation, PNA-guide strand duplex purified by HPLC (DNAPac P100, 50% water 50% acetonitrile, salt gradient 0-1M NaClO4). (B) Liver and kidney from mice injected with 40 mg/kg of either cholesterol, DCA, EPA, or DHA were used to quantify the guide strand after 48 hours, showing differential distribution of fatty acid conjugates.

FIG. 7 shows modified oligonucleotide sequences. Chemical modifications are abbreviated as follows, wherein “X” represents A, U, G, or C: fX (2′-fluoro), mX (2′-O-methyl), P (5′-phosphate), Chol (Cholesterol), ‘#’ (phosphorothioate backbone modification), ‘.’ (phosphodiester backbone).

FIG. 8 shows the solid-phase synthesis of DHA-conjugated hsiRNA.

FIG. 9 shows a representative LC-MS characterization of Cy3-DHA-hsiRNA^{HTT}; Calculated: 6174.1 for [M−H]−, found: 6174.4. Conditions: Buffer A: 15 mM Dibutylamine/25 mM HFIP, Buffer B: 20% A in MeOH, Column: xbidge OST C18, 2.5 um, Gradient: 0-10 min (1% B-80% B), 10-13 min (80% B-80% B), 13.1 min (80% B-1% B), 13.1-18 min (1% B-1% B).

FIG. 10 shows brain retention and distribution of g2DHA-hsiRNA.

FIG. 11A-C show the effects upon single IS injection of g2DHA-hsiRNA: (A) experimental procedure; (B) approximately 80% silencing in mouse striatum; (C) approximately 80% silencing in mouse cortex. There was no indication of toxicity and silencing was limited to injected side of the brain. hsiRNA-conjugate structures and modifications are shown in FIG. 5A-F.

FIG. 12 shows g2DHA support synthesis I.

FIG. 13 shows g2DHA support synthesis II (see Example 5).

FIG. 14 depicts exemplary values of X^{c}.

FIG. 15 depicts exemplary internucleotide linkages.

FIG. 16 depicts exemplary internucleotide backbone linkages.

FIG. 17 depicts exemplary sugar modifications.

FIG. 18A-E depict fully metabolically stabilized hsiRNAs (FM-hsiRNAs). (A) Schematics of partially and fully modified hsiRNAs. (B) hsiRNA and FM-hsiRNA have equal ability to enter RISC (HeLa, 72 hours, QuantiGene®). hsiRNA-conjugate structures, sequences, and modifications are found in FIG. 5A-F. (C) FM-hsiRNA, but not naked siRNA, supports passive delivery. (D) Metabolically stable 5′-E-VP (Vinylphosphonate) is as active as 5′-P (Phosphate). The antisense strand of the hsiRNAs are capped at the 5′ as follows: FM-hsiRNA-no P is capped with a 5′-OH; FM-hsiRNA is capped with a 5′ phosphate; FM-hsiRNA-EVP is capped with a 5′ vinyl phosphonate. (E) 5′-E-VP enables sustained delivery to distant tissues (7 days post injection, PNA assay). The antisense strand of the hsiRNAs are capped at the 5′ as follows: 5′P-hsiRNA is capped with a 5′ phosphate; 5VP′-hsiRNA is capped with a 5′ vinyl phosphonate. The hsiRNA sequence for FIG. 33 D-E is PPIB, found in FIG. 7.

FIG. 19 shows the optimized solution phase synthetic route to g2DHA-hsiRNA (1b). Reagents and conditions: (a) 20% piperidine in DMF (2×15 min); (b) 2-cyanoethyl NN-diisopropylchlorophosphoramidite, DIEA, DCM, 2 h, rt, 95%; (c) choline tosylate, ETT, MeCN, 2 h, rt, followed by mCPBA, 10 min, rt, 69%; (d) (e) TFA in dry DCM (1:1), triisopropylsilane, 2 h, rt then 10% diisopropylethylamine in MeCN, 1.5 h, rt 74% (f) 3, BOP, HOBt, DMF, 2,4,6-collidine, rt, 12 h; (g) 20% piperidine in DMF (2×15 min), rt; (h) DHA, HATU, DMF, rt, 12 h; (i) RNA synthesis, cleaving, deprotection, purification and ion-exchange. See also Example 6.

FIG. 20 shows the optimized solid-phase synthetic route to g2DHA-hsiRNA (1b). See also Example 7.

FIG. 21 shows that intravenous injection of lipid-siRNA conjugates induces differential levels of gene silencing in the liver, which is directly proportional to the degree of accumulation. Intravenous injection (20 mg/kg) of each siRNA conjugate. Animals sacrificed 7 days post-injection. Tissue punches taken from the liver tissue. mRNA was quantified using Affymetrix Quantigene 2.0 as per Coles et al. 2015. hsiRNA-conjugate structures and modifications are shown in FIG. 5A-F and the PPIB hsiRNA sequence is shown in FIG. 7.

FIG. 22A-B depicts targeted kidney delivery with polyunsaturated fatty acid chemical scaffolds. (A) Intravenous injection of PBS, Chol-siRNA, or g2DHA-siRNA (20 mg/kg twice daily for two days). Animals sacrificed 7 days post-injection. 63× image of kidney sections showing Cy3-fluorescence of oligonucleotides. hsiRNA-conjugate structures and modifications are shown in FIG. 5A-F. (B) siRNA antisense strands present in liver and kidney were quantified using Cy3-labeled complimentary PNA to hybridize to the strand and HPLC to quantify ng of oligo per mg of tissue. hsiRNA-conjugate structures and modifications are shown in FIG. 5A-F and the PPIB sequence is on FIG. 7.

FIG. 23 shows that g2DHA-hsiRNA preferentially distributes to proximal convoluted tubule cells throughout the kidney following systemic administration (two IV injections of 20 mg/kg, 48 hours). This sharply contrasts with the predominant liver localization exhibited by most siRNA therapeutics in the clinic and opens the window to expand the clinical utility of siRNA beyond liver indications.

FIG. 24 shows g2DHA-hsiRNA distributed to heart tissue following systemic administration (one intravenous injection, 10 mg/kg). These tissues are not typically accessed by therapeutic siRNAs following intravenous administration.

FIG. 25 shows g2DHA-hsiRNA distributed to muscle and fat tissue following systemic administration (one intravenous injection, 10 mg/kg). These tissues are not typically accessed by therapeutic siRNAs following intravenous administration. hsiRNA-conjugate structures and modifications are shown in FIG. 5A-F.

FIG. 26 shows Eicosapentanoic acid (EPA)-hsiRNA accumulation in the skin following subcutaneous injection. This can be directly compared to cholesterol-conjugated hsiRNA, which accumulates to a greater degree around the site of injection. This higher degree of accumulation may cause local toxicity and adverse effects, which is well documented for intrastriatal (CNS) administration. hsiRNA-conjugate structures and modifications are found in FIG. 5A-F.

FIG. 27 shows that subcutaneous injection of EPA-hsiRNA induces gene silencing in the skin. Subcutaneous injection (40 mg/kg) EPA-siRNA. Animals sacrificed 7 days post-injection. Tissue punches taken from the center (skin from head to the center of the back), middle (skin around the midpoint of the animal), and tail skin. mRNA was quantified using Affymetrix Quantigene 2.0 as per Coles et al. 2015. hsiRNA sequence PPIB is found in FIG. 7.

FIG. 28A-B show that a single injection of DHA- or g2DHA-siRNA is detected in both the striatum (A) and cortex (B) on the injected side. Alternative methods of injection including intracerebroventricular may also facilitate bilateral distribution with only one injection. Intrastriatal injection 2-4 nmols DHA- or g2DHA-siRNA. Animals sacrificed 7 days post-injection. Tissue punches taken from the 300 um brain slices from the striatum and cortex. siRNA antisense strands present in different brain regions were quantified using Cy3-labeled complimentary PNA to hybridize to the strand and HPLC to quantify ng of oligo per mg of tissue. aCSF—Artificial CSF. hsiRNA-conjugate structures and modifications are found in FIG. 5A-F and the PPIB sequence is shown in FIG. 7.

FIG. 29 shows serum lipoprotein binding properties of lipid-conjugated siRNAs.

FIG. 30 shows the lipoprotein profile of FVB/NJ mice. Whole mouse blood (˜500 μL) was collected in a sterile EDTA-coated tube following cardiac puncture. Samples were spun at 10,000 RPM for 10 minutes. 50 μL of serum was directly injected on Superose 360 size exclusion column. Fractions were collected over 300 minutes and analyzed for cholesterol content by the HDL/LDL Cholesterol Assay Kit (Abcam).

FIG. 31A-B depict serum lipoprotein profile analysis of siRNA in mouse blood. (A) cholesterol, DCA, and GM1 conjugates preferentially associate with IDL and LDL. hsiRNA-conjugate structures and modifications are found in FIG. 5A-F. (B) EPA, DHA, and DHAg2 conjugates preferentially associate with HDL. The structure of the EPA conjugate can be found in FIG. 41. hsiRNA conjugates (15 μM) were incubated in 50 μL of serum at room temperature for 30 minutes. 50 μL of serum was directly injected on Superose 360 size exclusion column. Fractions were collected over 300 minutes and analyzed for cholesterol content by the HDL/LDL Cholesterol Assay Kit (Abcam). The HTT sequence is shown in FIG. 7.

FIG. 32A-B show chemical structures of novel hydrophobic siRNA constructs. Polyunsaturated fatty acids are typically circulated in the bloodstream in an esterified form, meaning they are linked to glycerol, long-chain aliphatic alcohols, amides, phosphatidylcholine, phosphatidylserine, phosphoric acid, and phosphatidylethanolamine, among others. Defining the path to synthesize metabolically stable analogs of these naturally existing circulating compounds is one way to improve polyunsaturated fatty acid-siRNA tissue distribution and cellular uptake. (A) A generic hydrophobic siRNA construct where X is a hydrophobic lipid bioconjugate (e.g. polyunsaturated fatty acid, cholesterol). Y is a chemically stable trifunctional spacer or linker, which could be cleavable or not. Z is a naturally occurring ester linkage (e.g. phosphatidycholine, phosphatidylserine, phosphoric acid, see FIG. 32B)

FIG. 33 shows examples of linkers, spacer, and branching moieties. The exact chemical composition of the linker is not essential for activity as long as the branching structure can be generated

FIG. 34 shows hydrophobic siRNA conjugates with esterified phosphatidylcholine modifiers.

FIG. 35A-B show hsiRNA conjugates association with different lipoprotein particles.

FIG. 36 shows the route of synthesis of g2 conjugated CPG.

FIG. 37 shows exemplary lipophilic conjugates in both g1 (without a Zc modifier) and g2 (with a Zc modifier) forms. Shown are docosahexaenoic acid (“DHA” or “DHA g1”), DHA g2 (also referred to herein as “PC-DHA”), docosanoic acid (“DCA” or “DCA g1”), DCA g2 (also referred to herein as “PC-DCA”), eicosapentaenoic acid (“EPA” or “EPA g1”), EPA g2 (also referred to herein as “PC-EPA”), cholesterol g1, cholesterol C7 g1, cholesterol g2, lithocholic acid (“LA” or “LA g1”), LA g2 (also referred to herein as “PC-LA”), retinoic acid (“vitamin A,” “RA” or “RA g1”), RA g2 (also referred to herein as “PC-RA”), a-tocopherol succinate (“vitamin E,” “TOCO” or “TOCO g1”), and TOCO g2 (also referred to herein as “PC-TOCO”).

FIG. 38 shows reverse phase HPLC data of conjugated Cy3 HTT sense strand oligonucleotides with various exemplary conjugates.

FIG. 39 shows altered liver/kidney distribution of siRNAs conjugated to various exemplary lipophilic moieties.

FIG. 40 shows fluorescent images of liver/kidney distribution of various exemplary siRNA conjugates.

FIG. 41 shows the altered tissue distribution of siRNA conjugates.

FIG. 42 shows preferential delivery to podocytes in the Bowman's capsule of the kidney with Retinoic Acid (RA)-hsiRNA.

FIG. 43A-D show NMR and HRMS characterization of a synthesis intermediate shown in FIG. 36.

FIG. 44A-D show NMR and HRMS characterization of a synthesis intermediate shown in FIG. 36.

FIG. 45A-D show NMR and HRMS characterization of a synthesis intermediate shown in FIG. 36.

FIG. 46A-C show data for HTT Cy3 DHAg2 conjugates (FIG. 46A), HTT Cy3 LCAg2 conjugates (FIG. 46B), and HTT Cy3 retinoic acid g2 conjugates (FIG. 46C).

FIG. 47A-B show PNA assays depicting the effects of conjugate modality on hsiRNA tissue distribution 48 hours after a 20 mg/kg subcutaneous injection using g1 and g2 conjugates for DHA, EPA, DCA, RA, TOCO (α-tocopherol succinate), choline, LA (lithocholic acid) and cholesterol, or using unconjugated hsiRNA. (n=3.)

FIG. 48 depicts efficacy of HTT mRNA silencing in liver using various lipid conjugated hsiRNAs. Samples were analyzed 1 week post-subcutaneous injection of 20 mg/kg. (PPIB mRNA is non-targeting control; n=8 mice; normalization to PBS; one-way ANOVA comparison.)

FIG. 49 depicts efficacy of PPIB mRNA silencing in liver using various lipid conjugated hsiRNAs. Samples were analyzed 1 week post-subcutaneous injection of 20 mg/kg. (HTT mRNA is non-targeting control; n=8 mice; normalization to PBS; one-way ANOVA comparison.)

FIG. 50 depicts efficacy of HTT mRNA silencing in kidney cortex using various lipid conjugated hsiRNAs. Samples were analyzed 1 week post-subcutaneous injection of 20 mg/kg. (PPIB mRNA is non-targeting control; n=8 mice; normalization to PBS; one-way ANOVA comparison.)

FIG. 51 depicts efficacy of PPIB mRNA silencing in kidney cortex using various lipid conjugated hsiRNAs. Samples were analyzed 1 week post-subcutaneous injection of 20 mg/kg. (HTT mRNA is non-targeting control; n=8 mice; normalization to PBS; one-way ANOVA comparison.)

FIG. 52 depicts efficacy of HTT mRNA silencing in adrenal gland using various lipid conjugated hsiRNAs. Samples were analyzed 1 week post-subcutaneous injection of 20 mg/kg. (PPIB mRNA is non-targeting control; n=8 mice; normalization to PBS; one-way ANOVA comparison.)

FIG. 53 depicts efficacy of PPIB mRNA silencing in adrenal gland using various lipid conjugated hsiRNAs. Samples were analyzed 1 week post-subcutaneous injection of 20 mg/kg. (HTT mRNA is non-targeting control; n=8 mice; normalization to PBS; one-way ANOVA comparison.)

FIG. 54 depicts efficacy of HTT mRNA silencing in heart using various lipid conjugated hsiRNAs. Samples were analyzed 1 week post-subcutaneous injection of 20 mg/kg. (PPIB mRNA is non-targeting control; n=8 mice; normalization to PBS; one-way ANOVA comparison.)

FIG. 55 depicts efficacy of PPIB mRNA silencing in heart using various lipid conjugated hsiRNAs. Samples were analyzed 1 week post-subcutaneous injection of 20 mg/kg. (HTT mRNA is non-targeting control; n=8 mice; normalization to PBS; one-way ANOVA comparison.)

FIG. 56 depicts efficacy of HTT mRNA silencing in spleen using various lipid conjugated hsiRNAs. Samples were analyzed 1 week post-subcutaneous injection of 20 mg/kg. (PPIB mRNA is non-targeting control; n=8 mice; normalization to PBS; one-way ANOVA comparison.)

FIG. 57 depicts efficacy of PPIB mRNA silencing in spleen using various lipid conjugated hsiRNAs. Samples were analyzed 1 week post-subcutaneous injection of 20 mg/kg. (HTT mRNA is non-targeting control; n=8 mice; normalization to PBS; one-way ANOVA comparison.)

FIG. 58 depicts efficacy of HTT mRNA silencing in lung using various lipid conjugated hsiRNAs. Samples were analyzed 1 week post-subcutaneous injection of 20 mg/kg. (PPIB mRNA is non-targeting control; n=8 mice; normalization to PBS; one-way ANOVA comparison.)

FIG. 59 depicts efficacy of PPIB mRNA silencing in lung using various lipid conjugated hsiRNAs. Samples were analyzed 1 week post-subcutaneous injection of 20 mg/kg. (HTT mRNA is non-targeting control; n=8 mice; normalization to PBS; one-way ANOVA comparison.)

FIG. 60 depicts efficacy of PPIB mRNA silencing in spleen using various lipid conjugated hsiRNAs. Samples were analyzed 1 week post-subcutaneous injection of 20 mg/kg. (HTT mRNA is non-targeting control; n=8 mice; normalization to PBS; one-way ANOVA comparison.)

### DETAILED DESCRIPTION

The present disclosure relates to conjugated oligoinucleotides that are completely stable and fully active. To identify chemical and biological properties that drive oligonucleotide (e.g., small RNA), tissue distribution and cellular uptake, these oligonucleotides (e.g., small RNAs), were conjugated to several naturally occurring bioactive steroids, endocannabinoid-like lipids, vitamins and nucleoside analogs. The resulting conjugates selectively delivered small RNAs to a range of tissues, including thymus, bladder, intestine, skin, bone marrow, placenta, adipose tissue, muscle, spleen, pancreas, lung, fallopian tube, adrenal gland, heart, liver and kidney.

The compositions described herein promote simple, efficient, non-toxic delivery of oligonucleotides (e.g, metabolically stable siRNA), and promote potent silencing of therapeutic targets in a range of tissues in vivo. Provided herein is a chemistry platform for targeting other tissues matching the performance and clinical impact of GalNAc conjugates in the liver. Several bio-active steroids endocannabinoid-like bioactive lipid conjugates and vitamin-based conjugates were screened and identified. These compounds show unprecedented distribution, neuronal uptake, efficacy, and lack of toxicity in several tissues, including thymus, bladder, intestine, skin, bone marrow, placenta, adipose tissue, muscle, spleen, pancreas, lung, fallopian tube, adrenal gland, heart, liver and kidney.

In certain aspects, the oligonucleotide conjugates of the invention were identified through a process involving: (1) providing a fully metabolically stable scaffolds (no RNA left); (2) selecting compounds which are biologically known to internalize inside the cells and identifying the ranges of hydrophobicities which allow efficient tissue distribution; (3) conjugating these hydrophobic compounds to the metabolically stable siRNAs; and (4) screening distribution, efficacy and toxicity in vivo. The discovery of the optimal range of hydrophobicity defines the chemical scaffold ranges expected to be efficacious. It was found that low hydrophobicity (cortisol like) was not sufficient to secure good tissue retention, whereas too much hydrophobicity (e.g., cholesterol) minimized distribution from the site of injection.

In a first aspect, provided herein is a compound of formula (1):

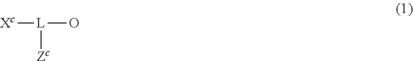

wherein:
O is a double-stranded nucleic acid comprising a first oligonucleotide and a second oligonucleotide, wherein:
   (1) the first oligonucleotide comprises at least 16 contiguous nucleotides, a 5′ end, a 3′ end and has complementarity to a target;
   (2) the second oligonucleotide comprises at least 15 contiguous nucleotides, a end, a 3′ end, and has homology with a target; and
   (3) a portion of the first oligonucleotide is complementary to a portion of the second oligonucleotide;
L is a divalent or trivalent linker;
   X^{c} is a hydrophobic moiety; and
   Z^{c} is a phosphodiester or phosphodiester derivative, or is absent.

### Variable L

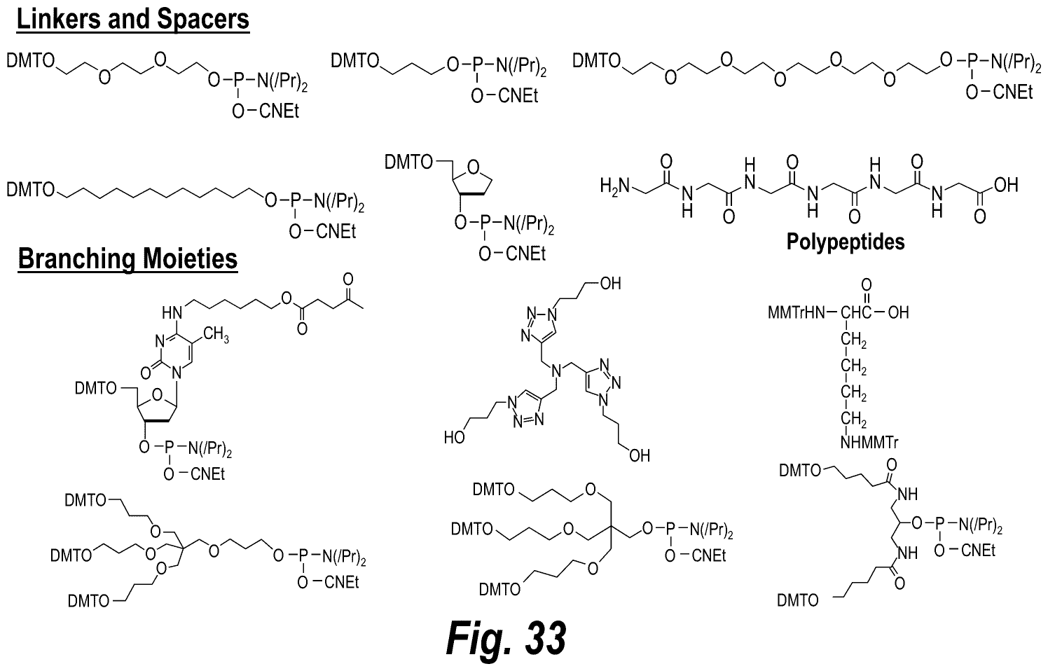

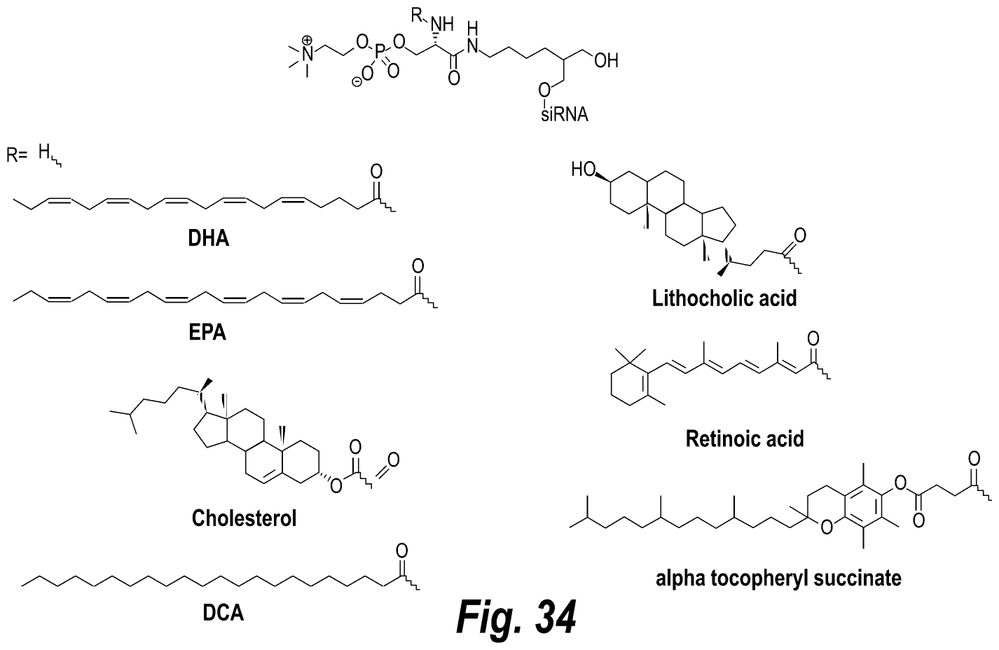

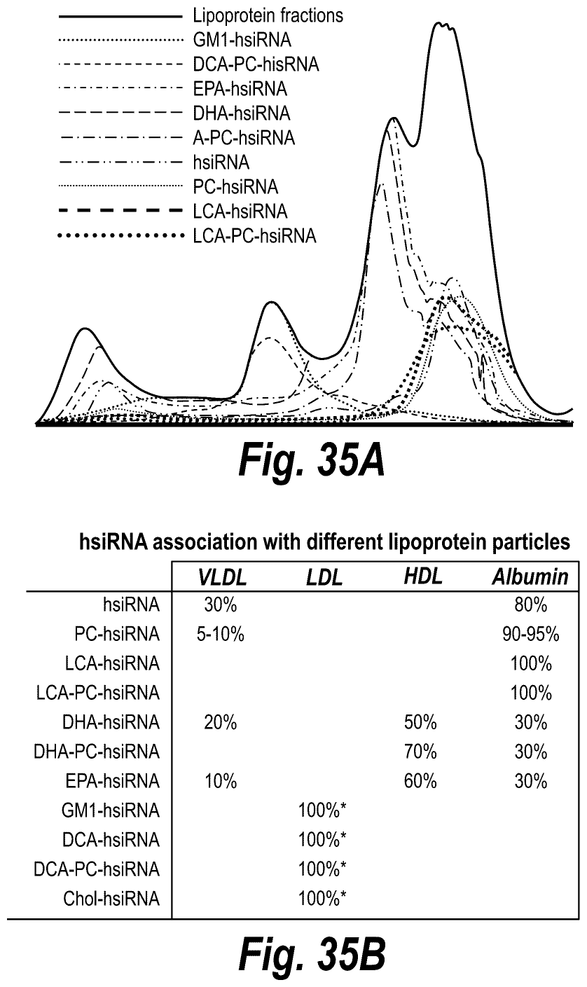

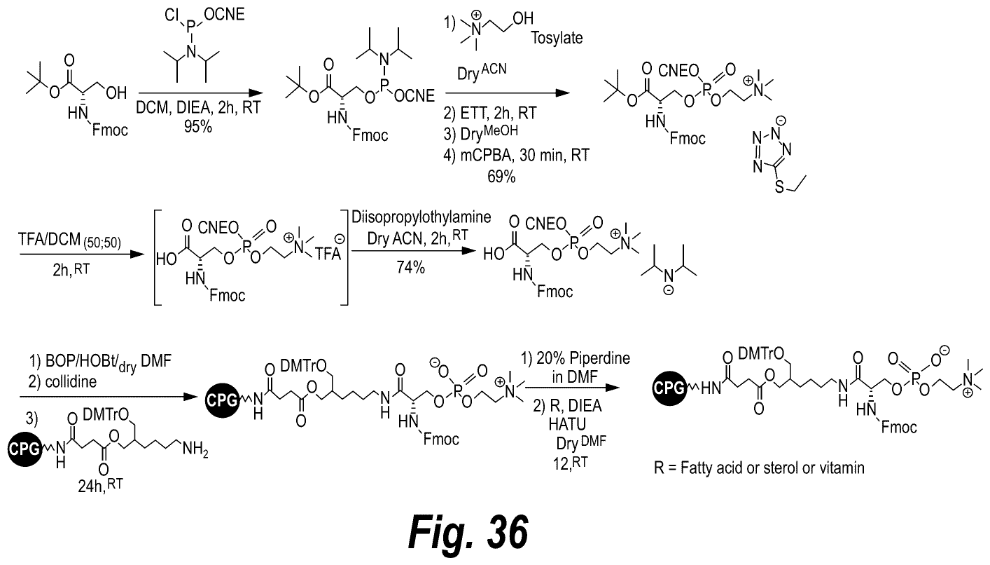

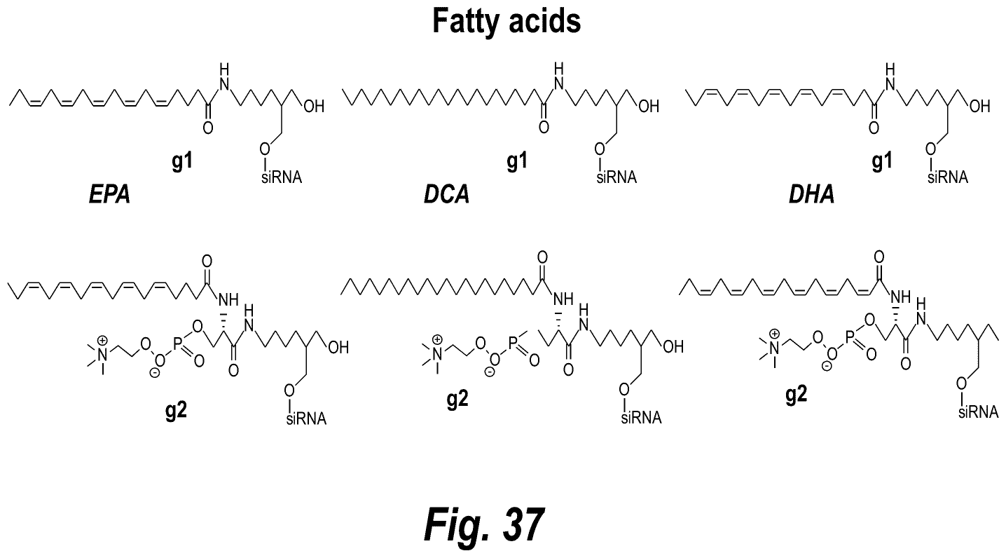

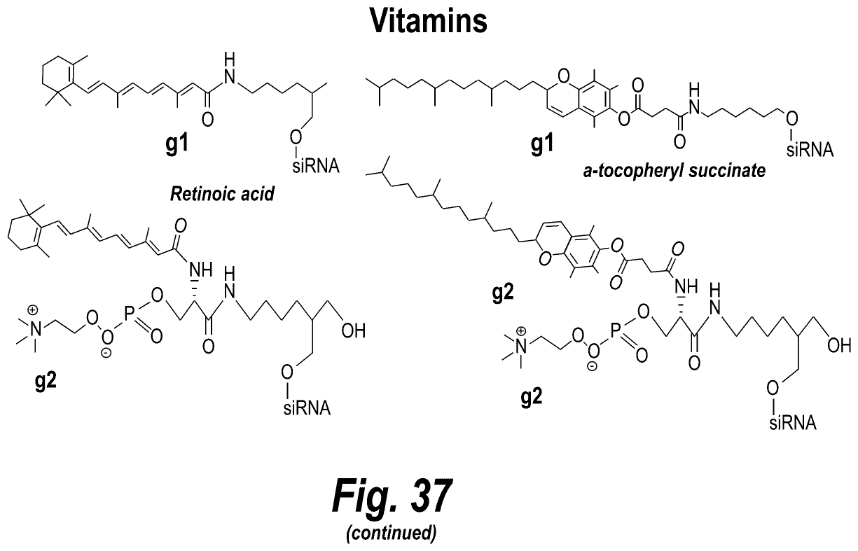

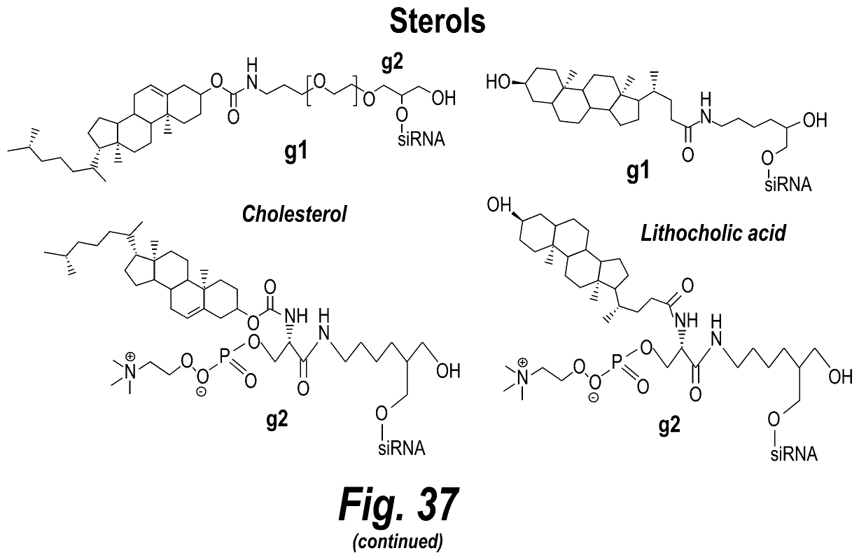

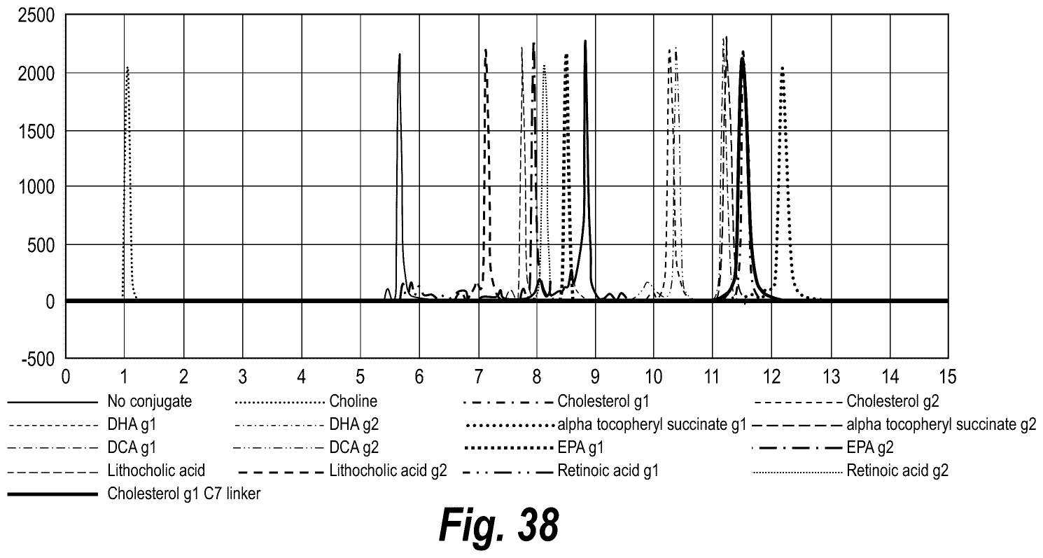

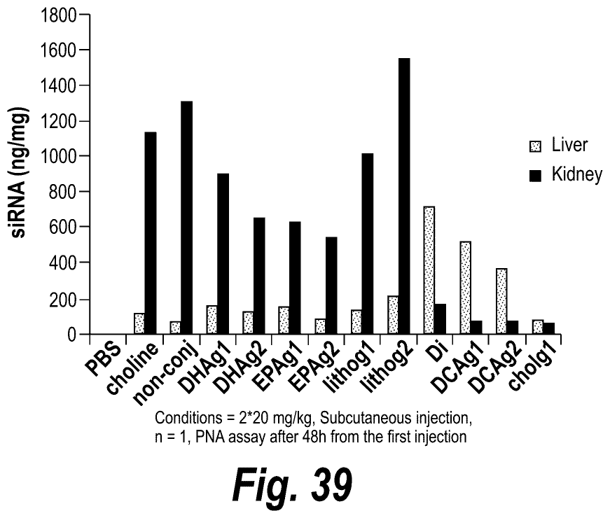

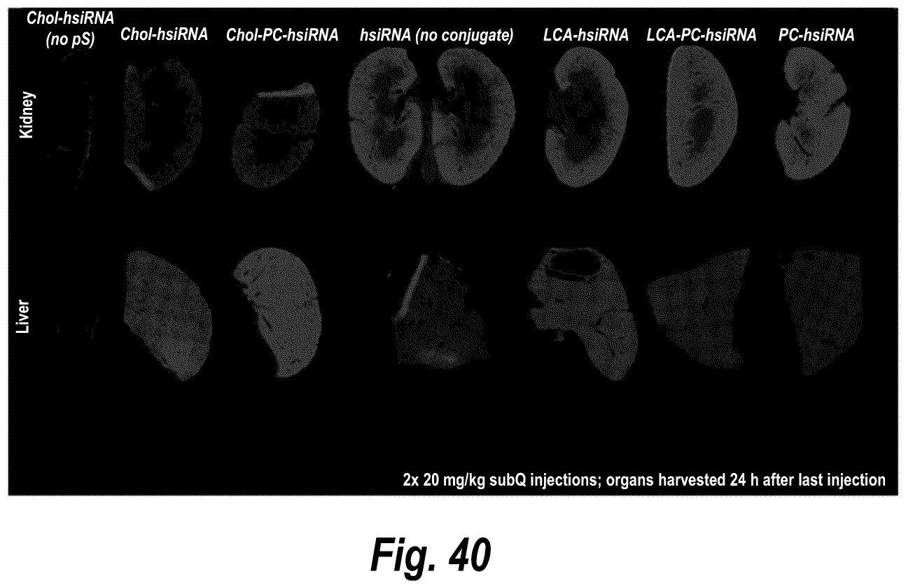

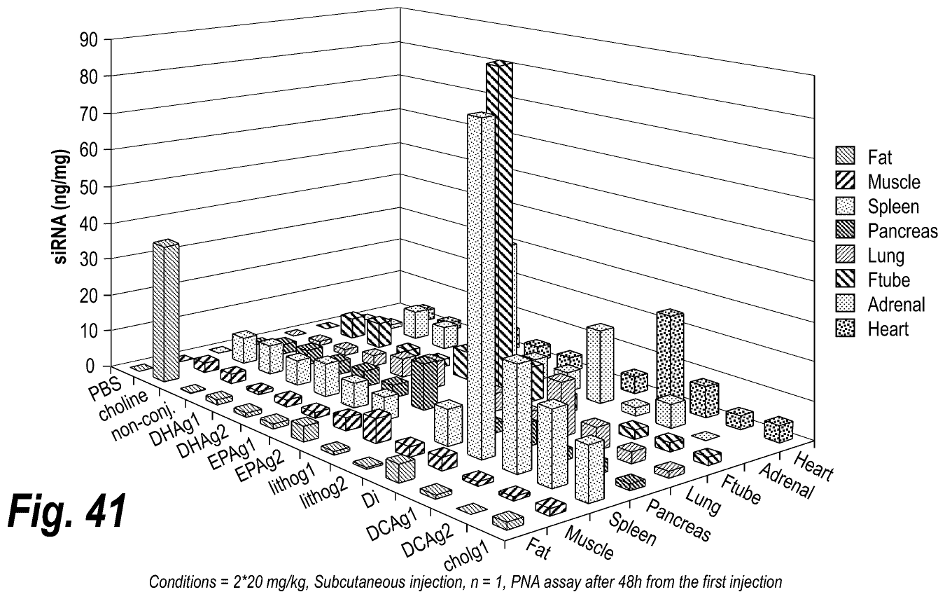

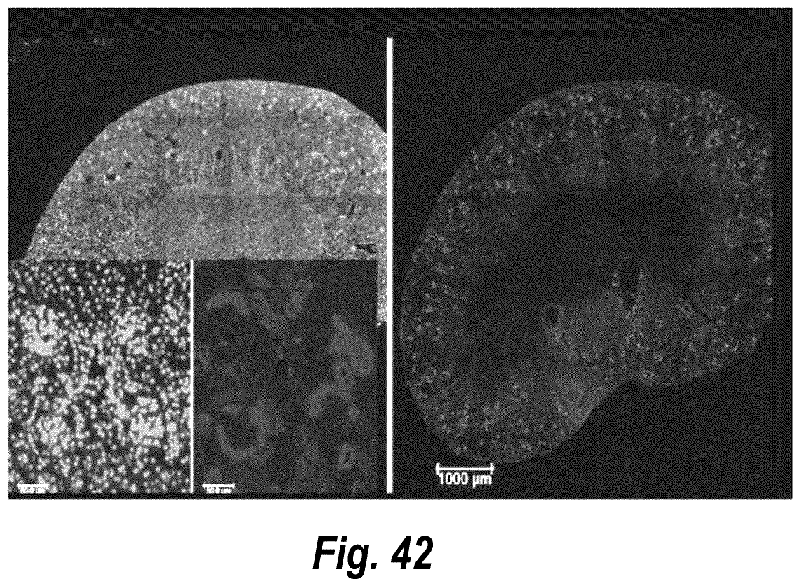

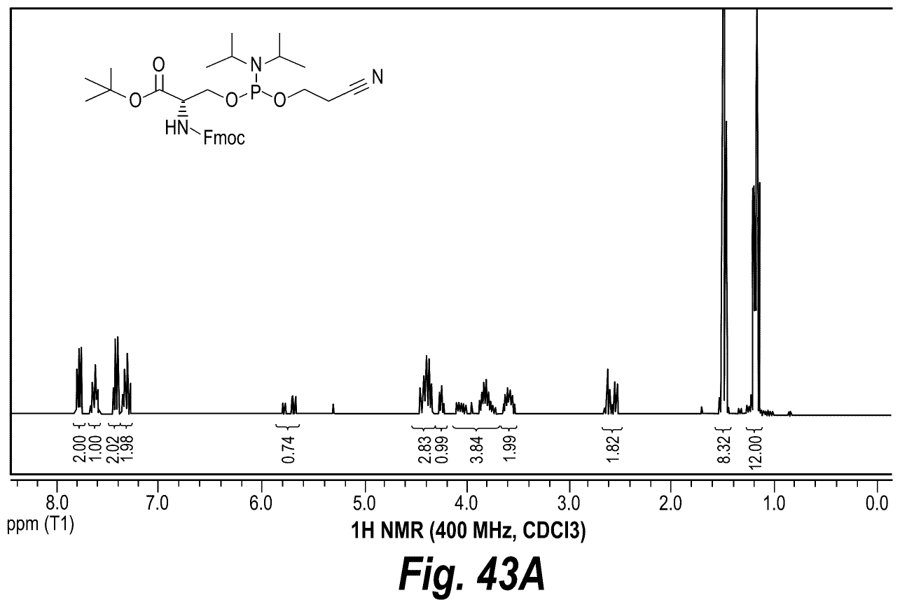

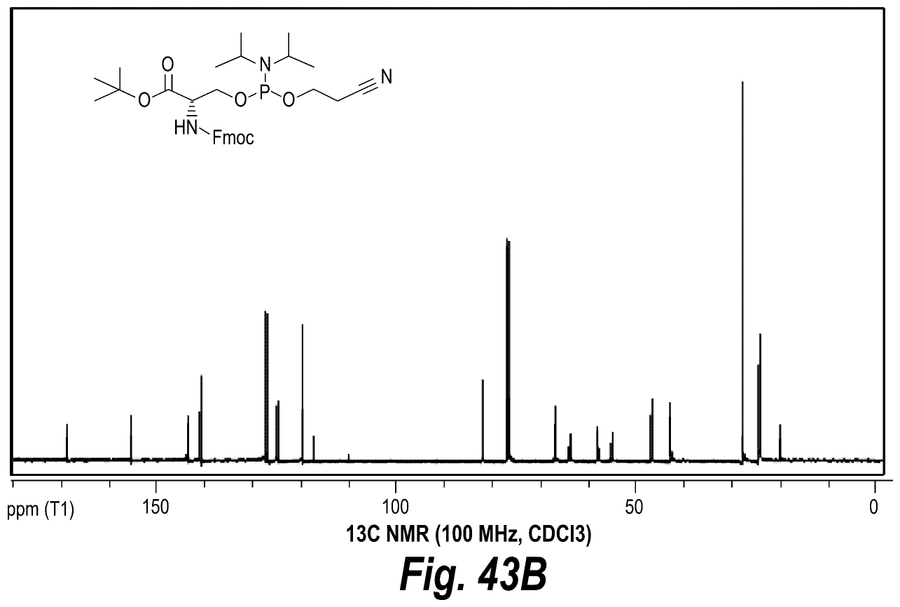

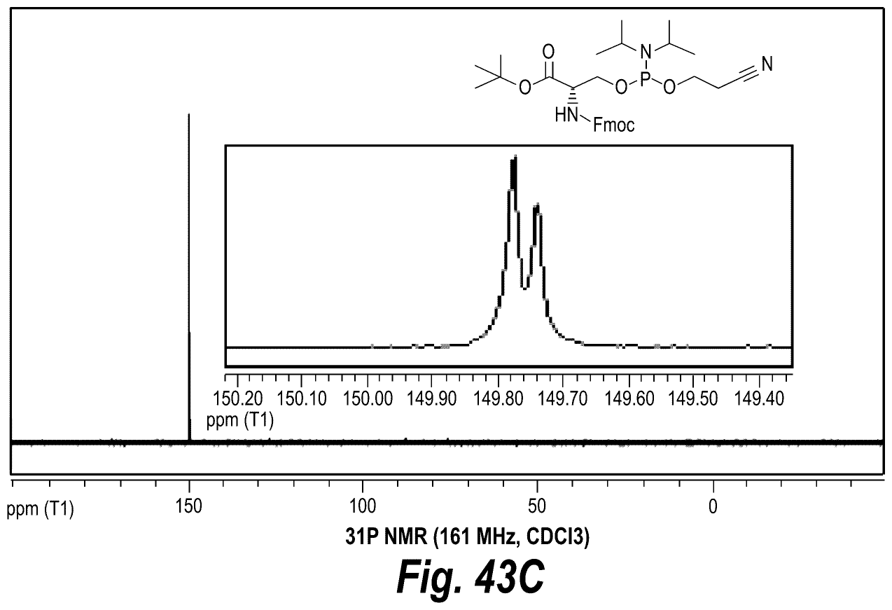

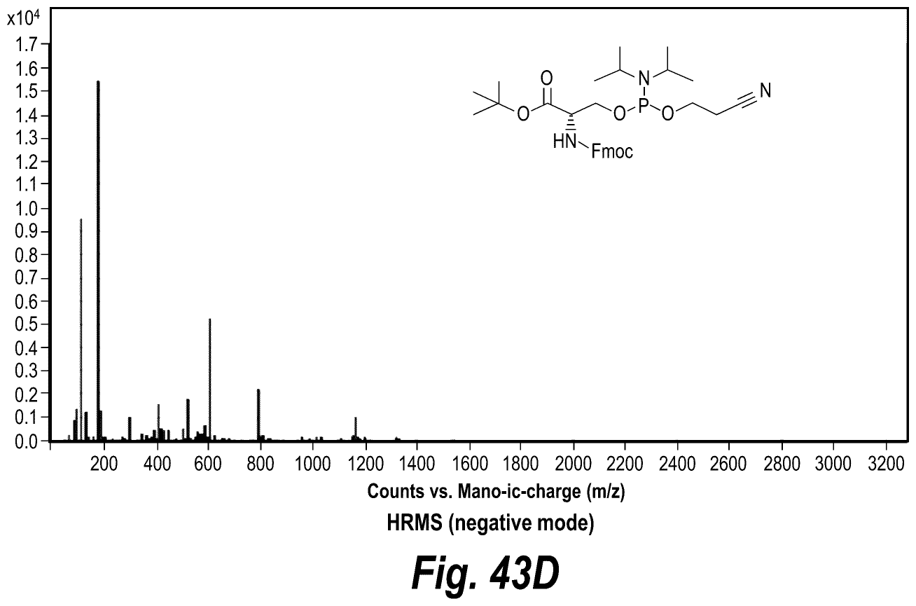

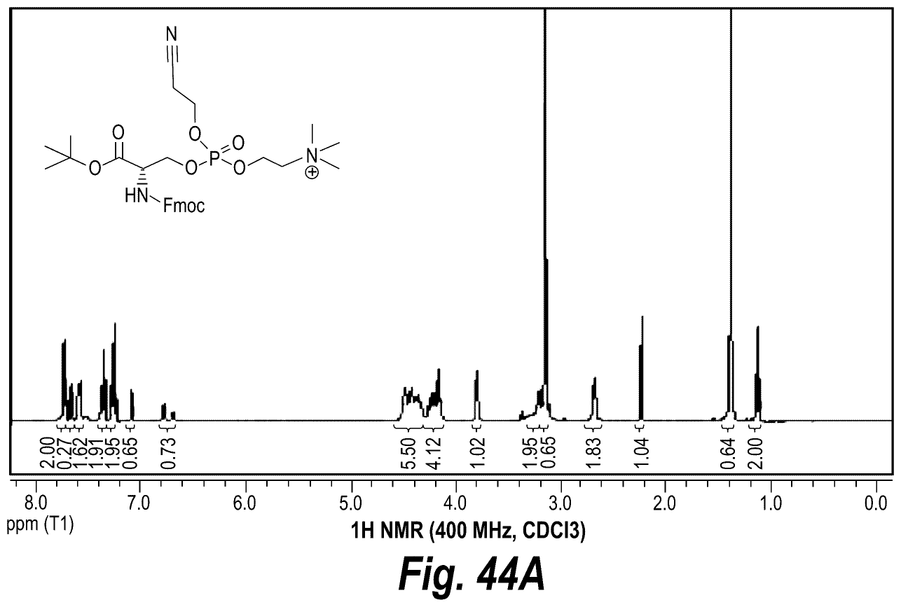

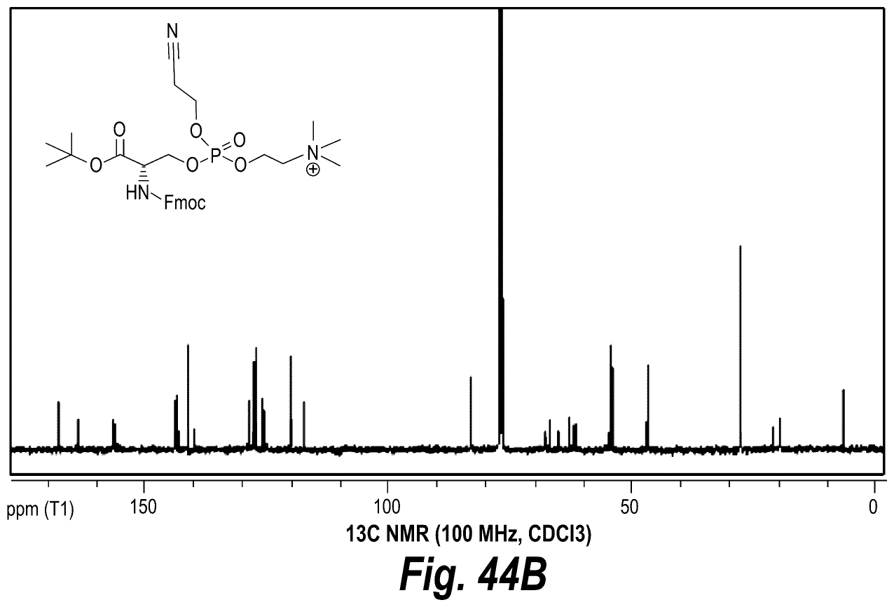

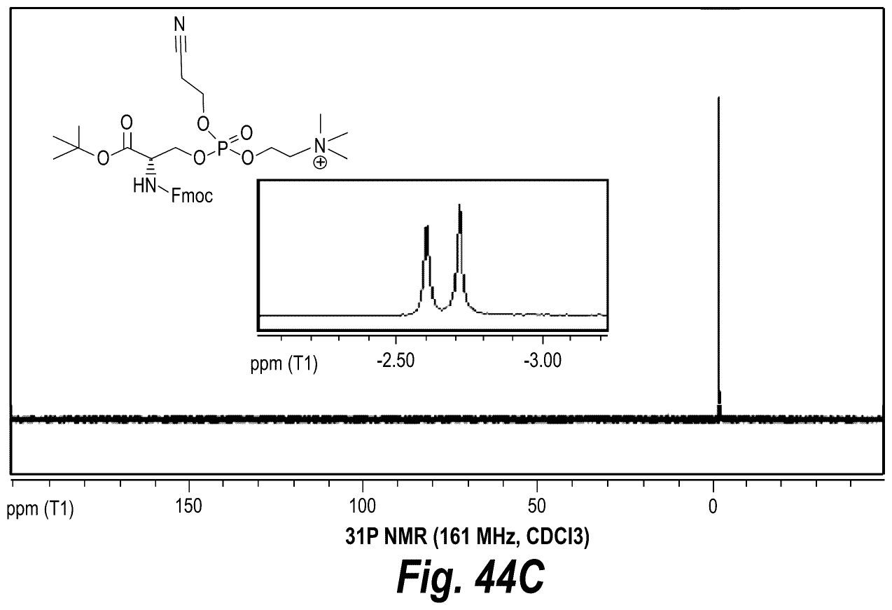

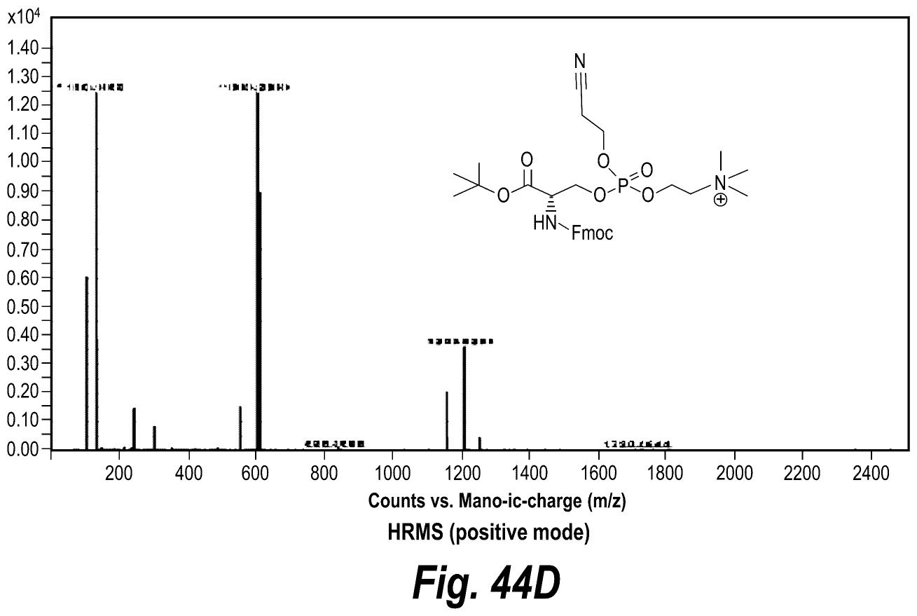

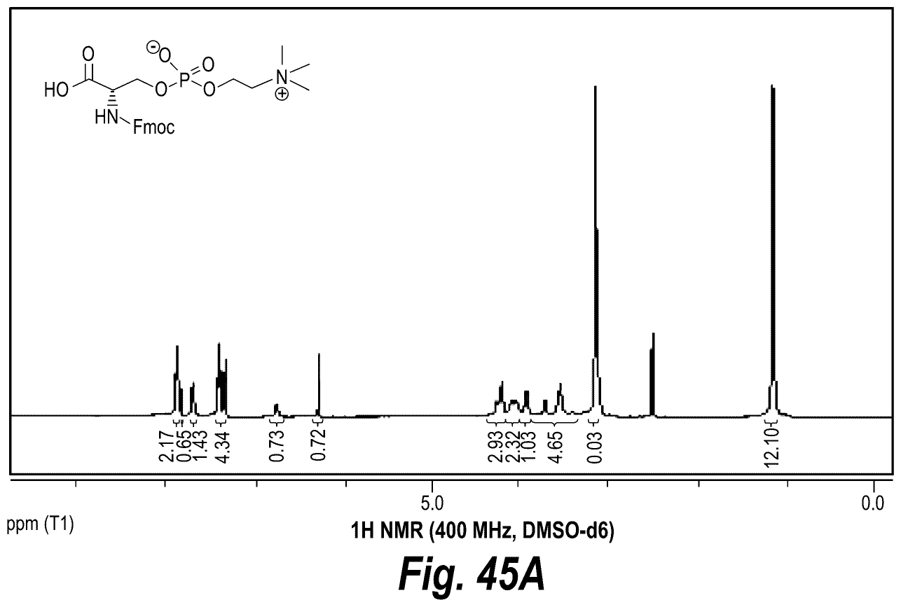

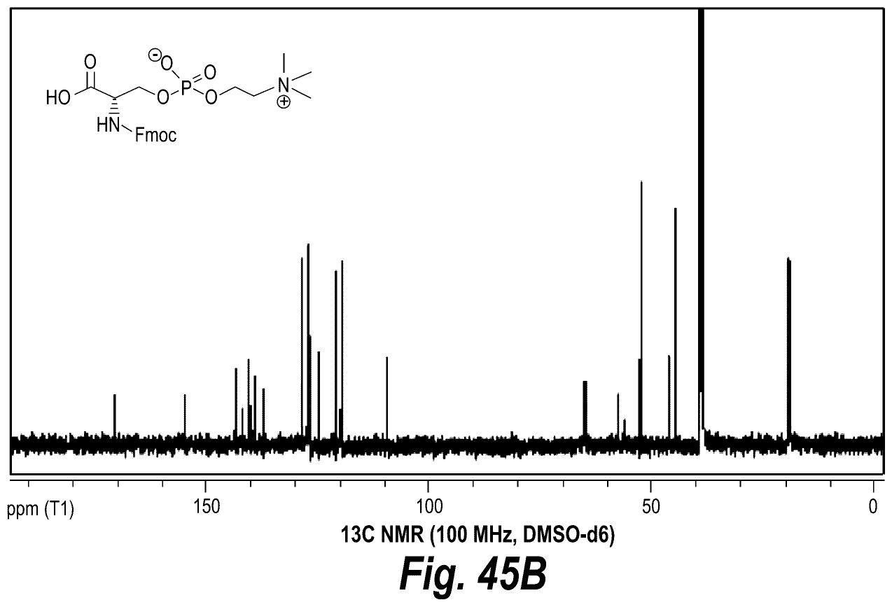

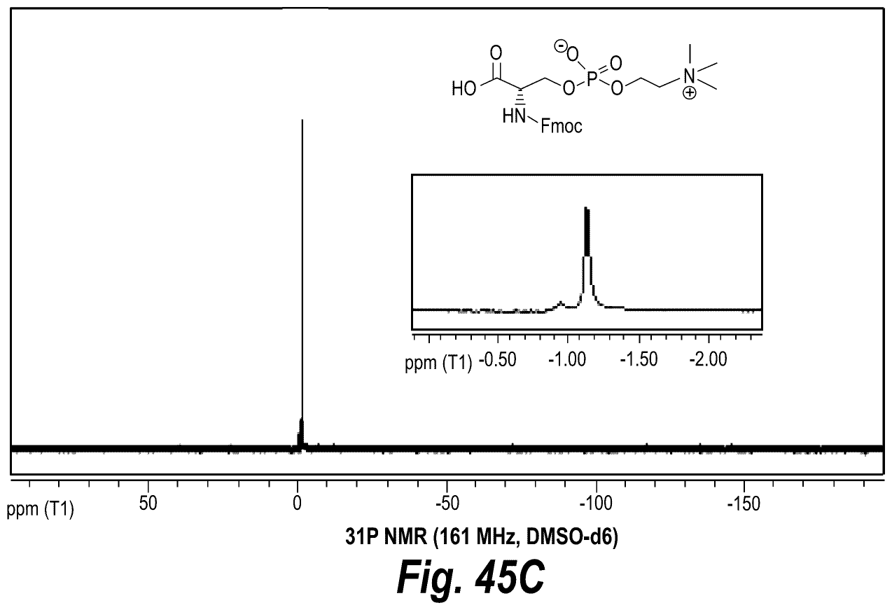

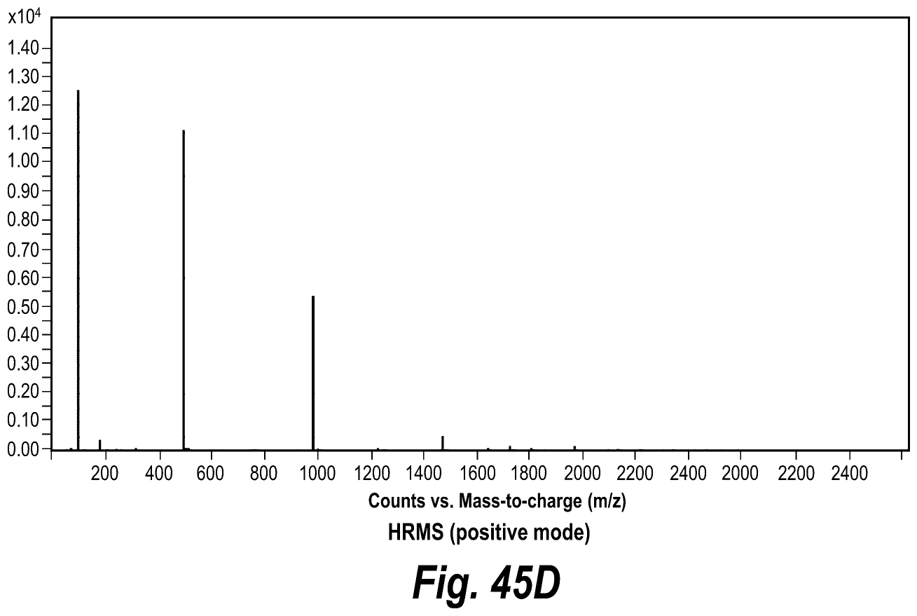

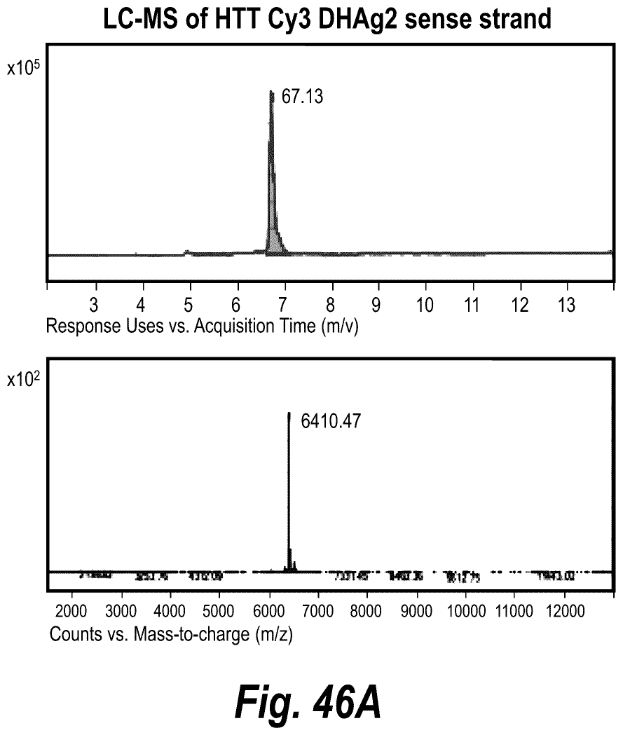

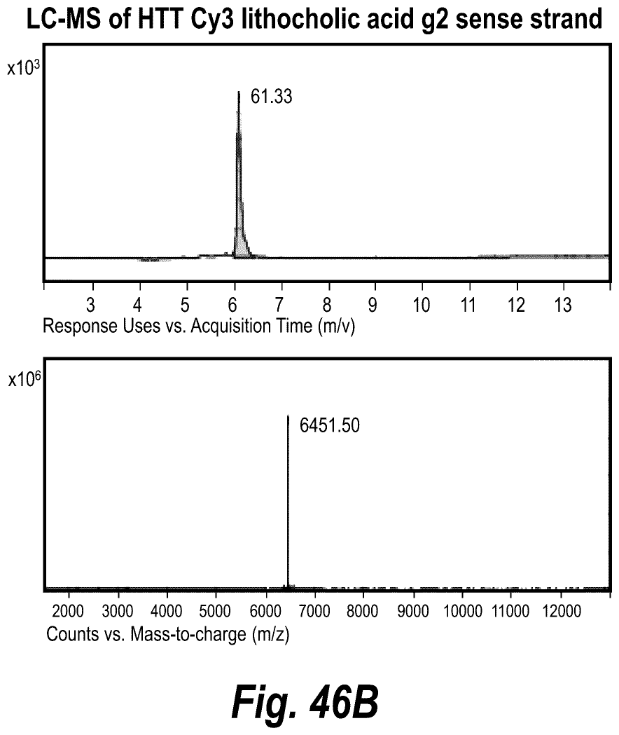

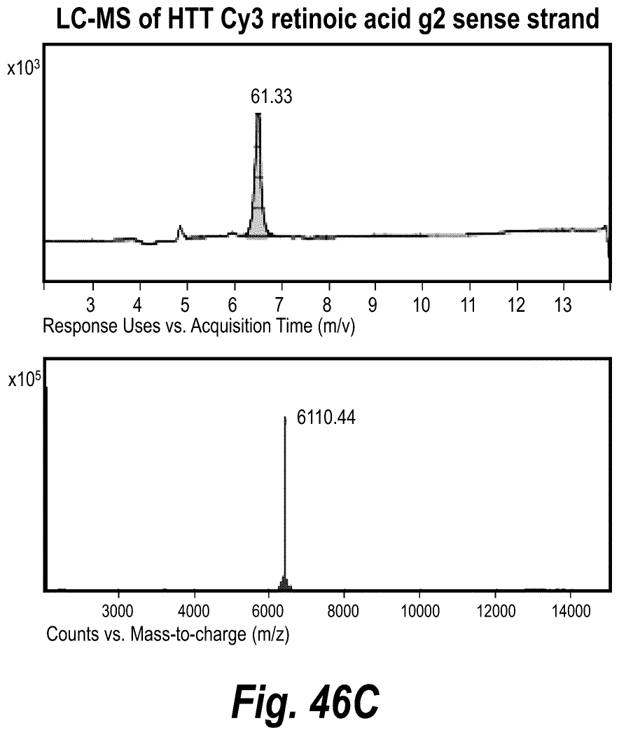

In one embodiment, L comprises an ethylene glycol chain, an alkyl chain, a peptide, RNA, DNA, a phosphodiester, a phosphorothioate, a phosphoramidate, an amide, a carbamate, or a combination thereof; and wherein L is attached to O via the second oligonucleotide. In one embodiment, L is a divalent linker. In another embodiment, L is a trivalent linker. In certain embodiments, L corresponds to a linker of FIG. 33.

In a particular embodiment, L is the trivalent linker L1, also referred to herein as C7:

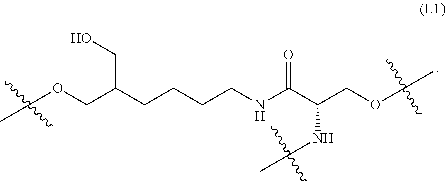 [SMILES: *N[C@@H](CO*)C(=O)NCCCCC(CO)CO*]

In another particular embodiment, L is the divalent linker L2:

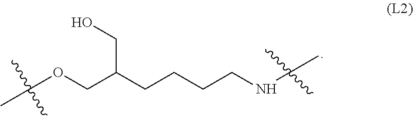 [SMILES: *NCCCCC(CO)CO*]

In another particular embodiment, L is a trivalent or bivalent linker selected from the group consisting of:

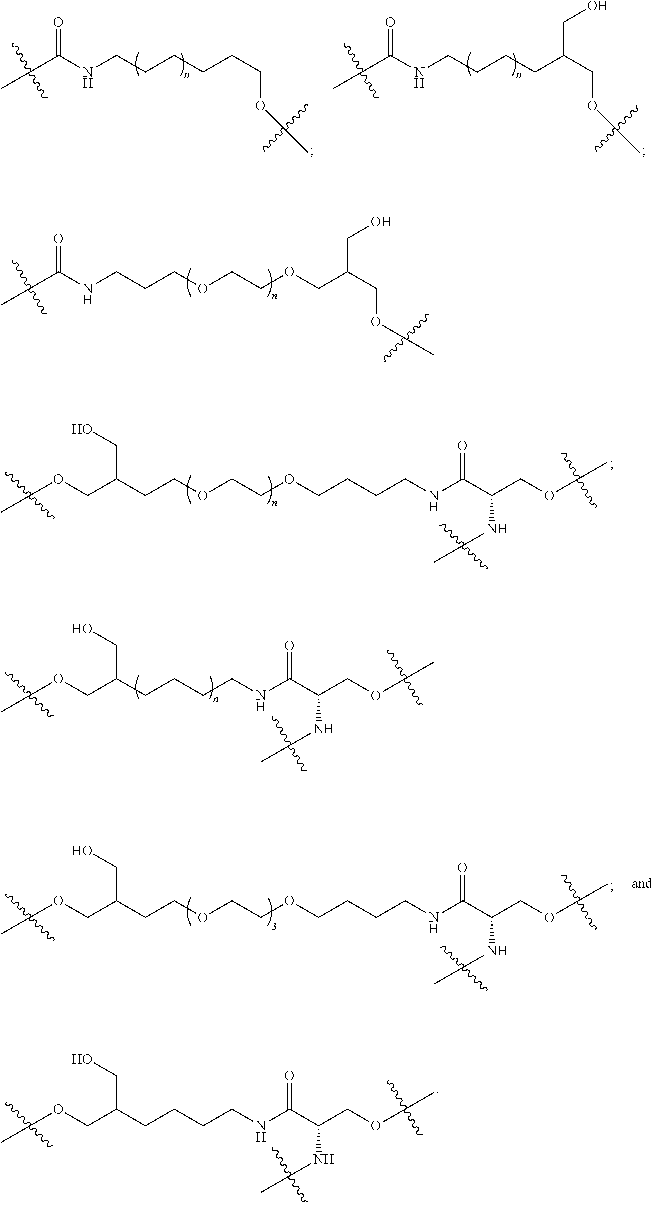

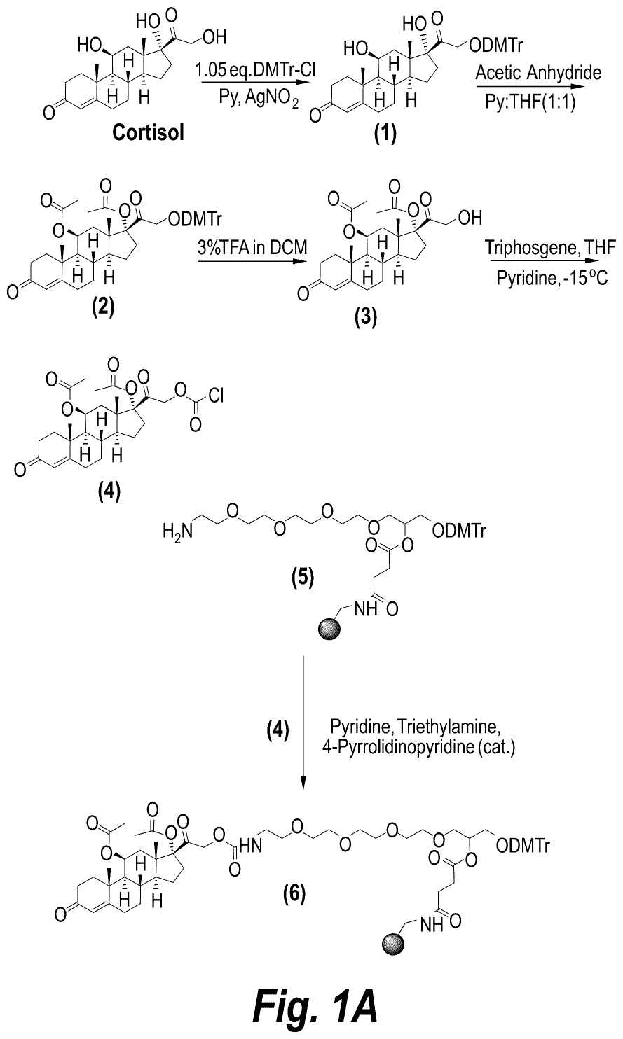

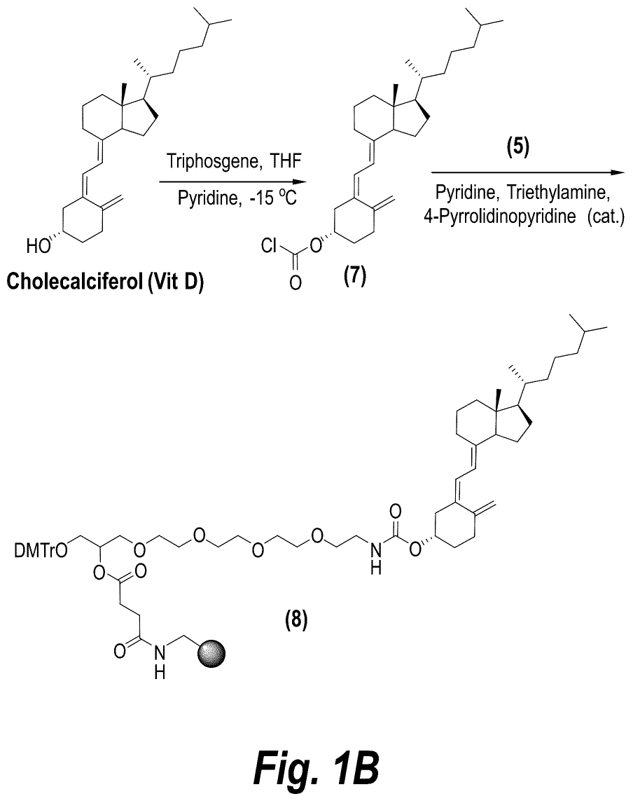

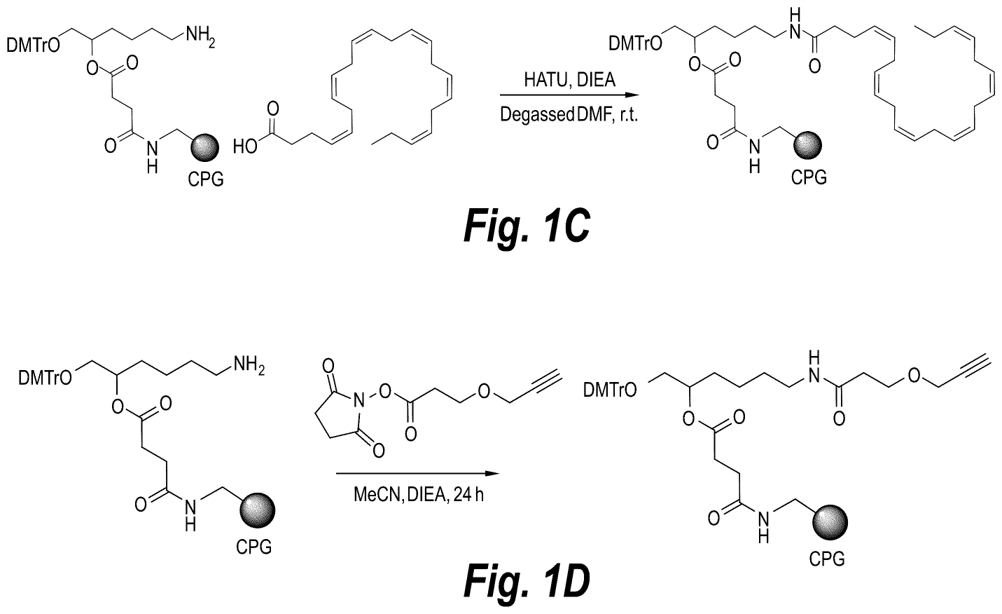

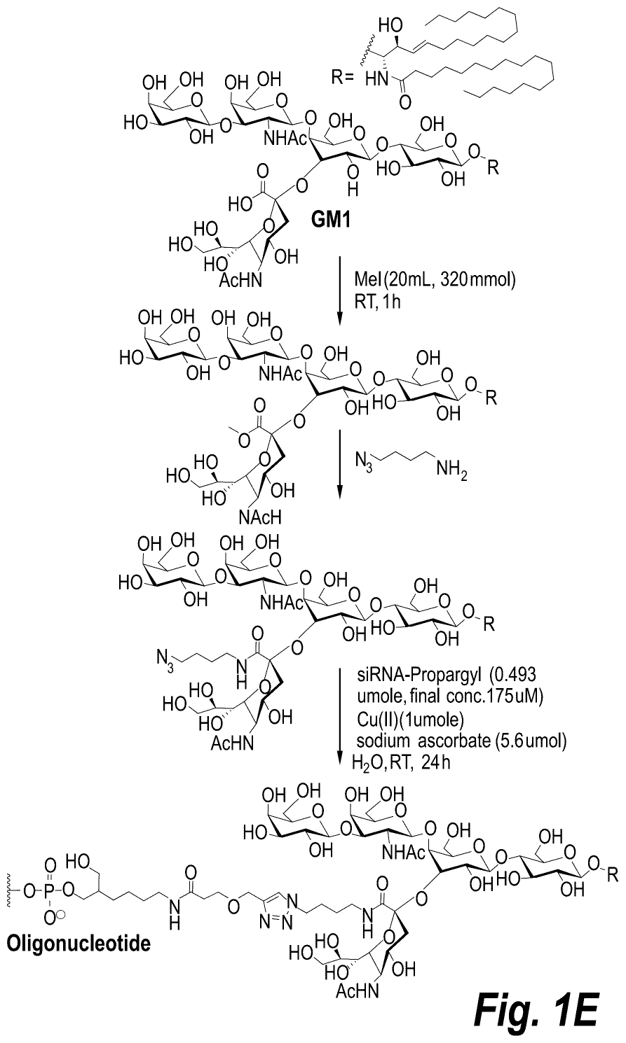

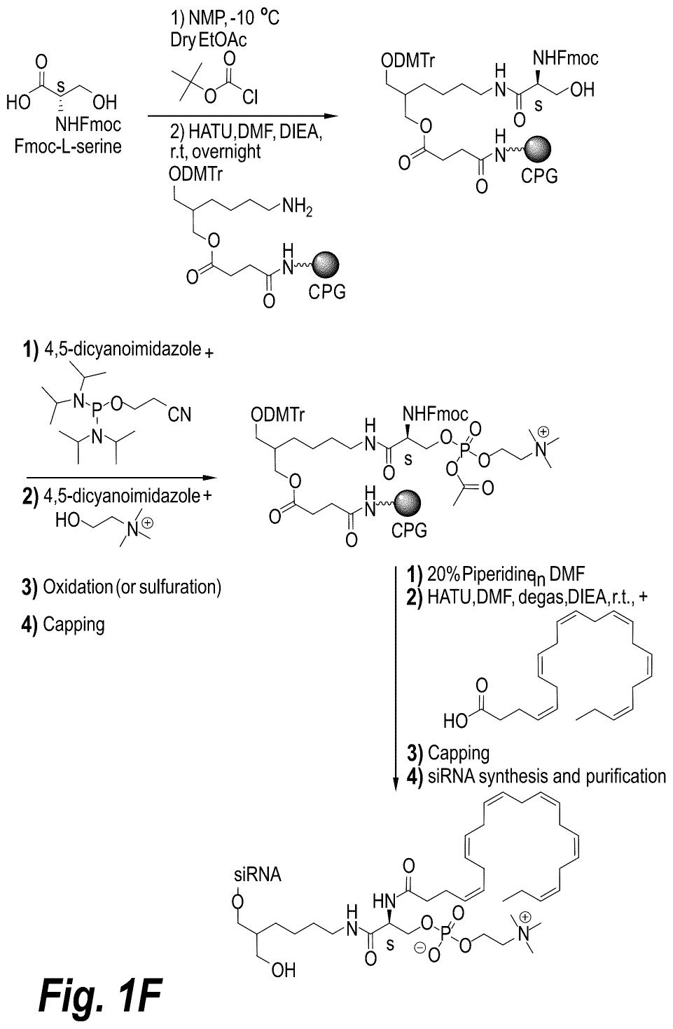

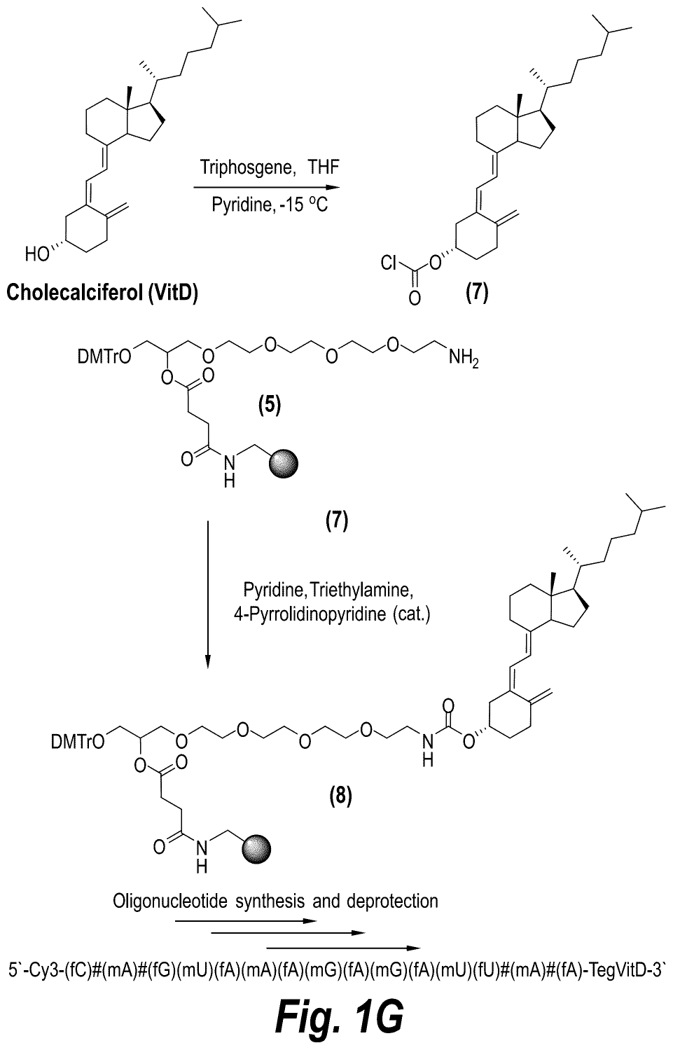

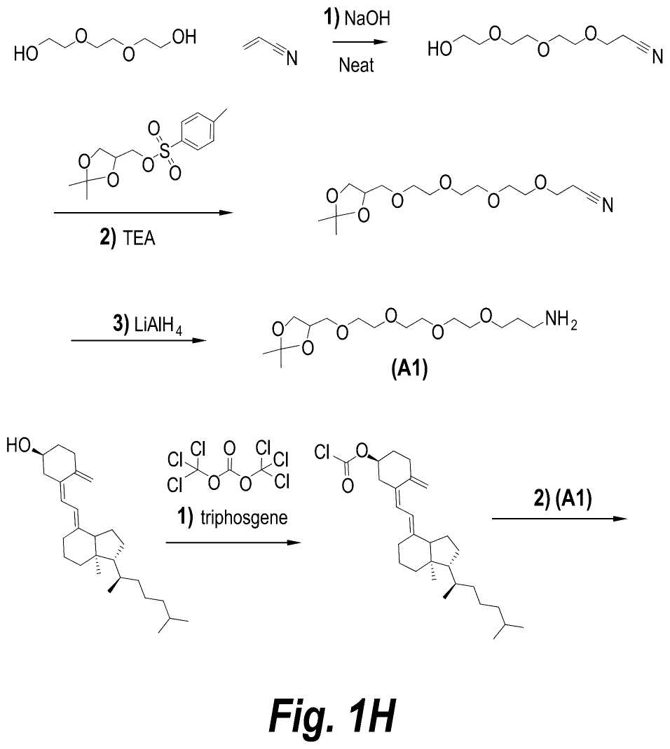

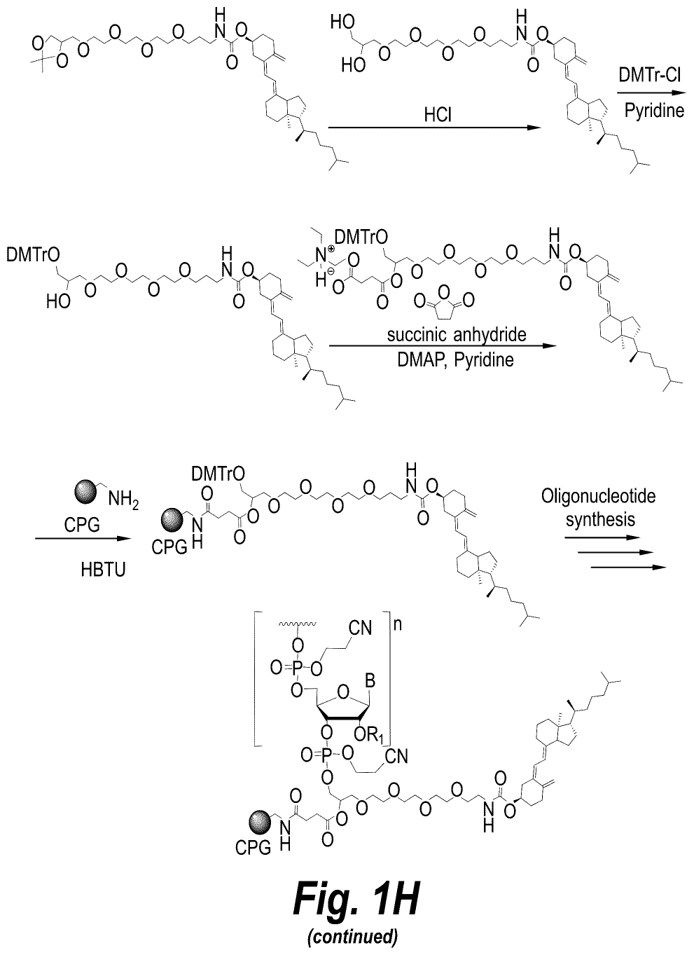

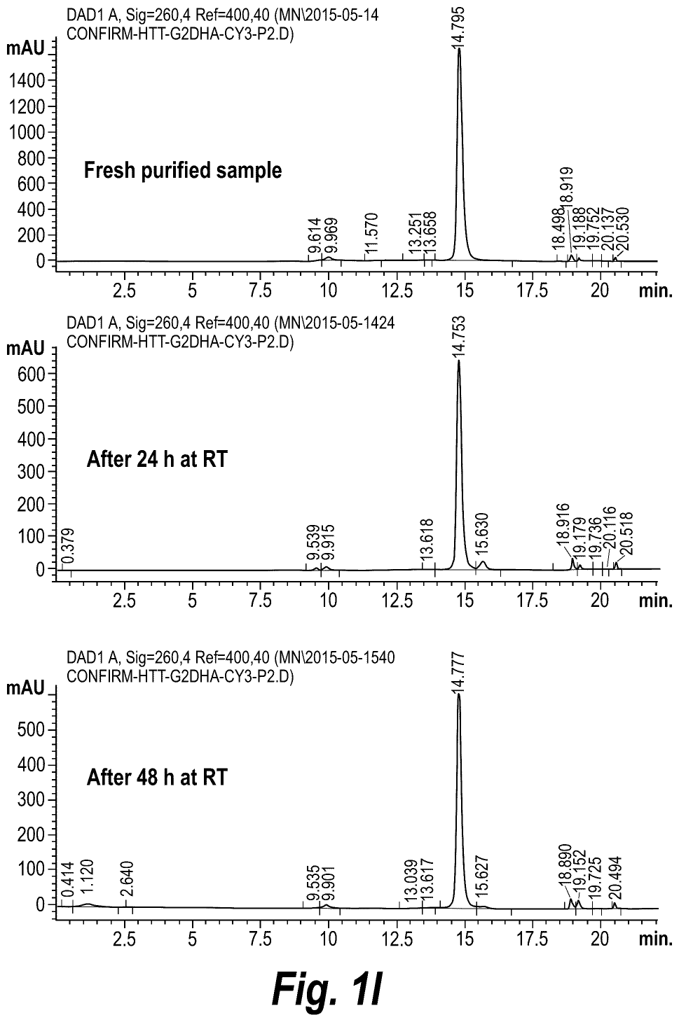

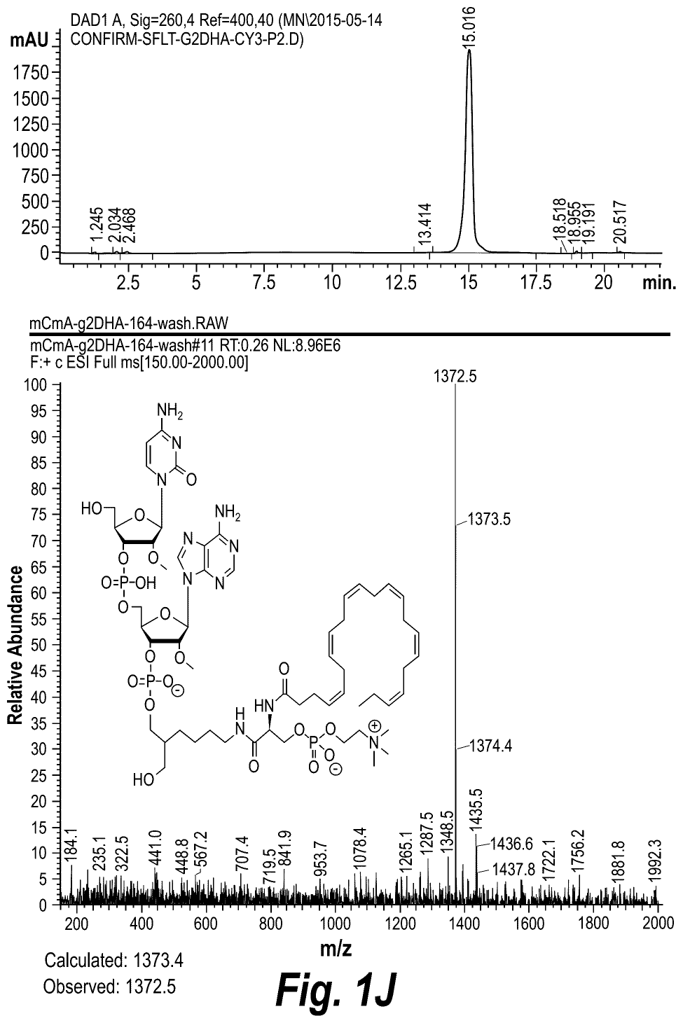

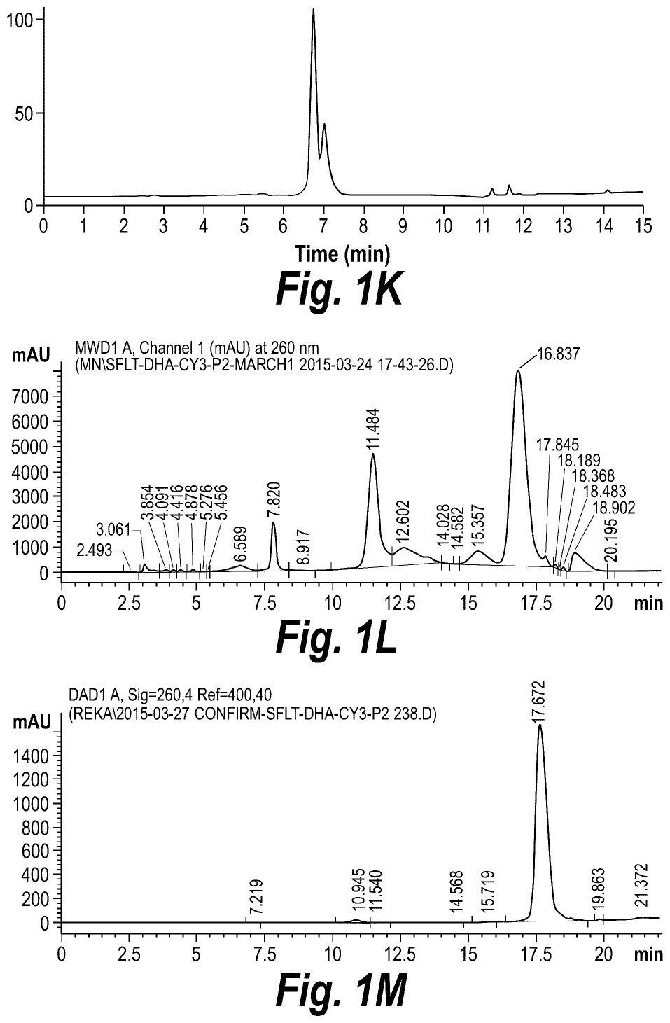

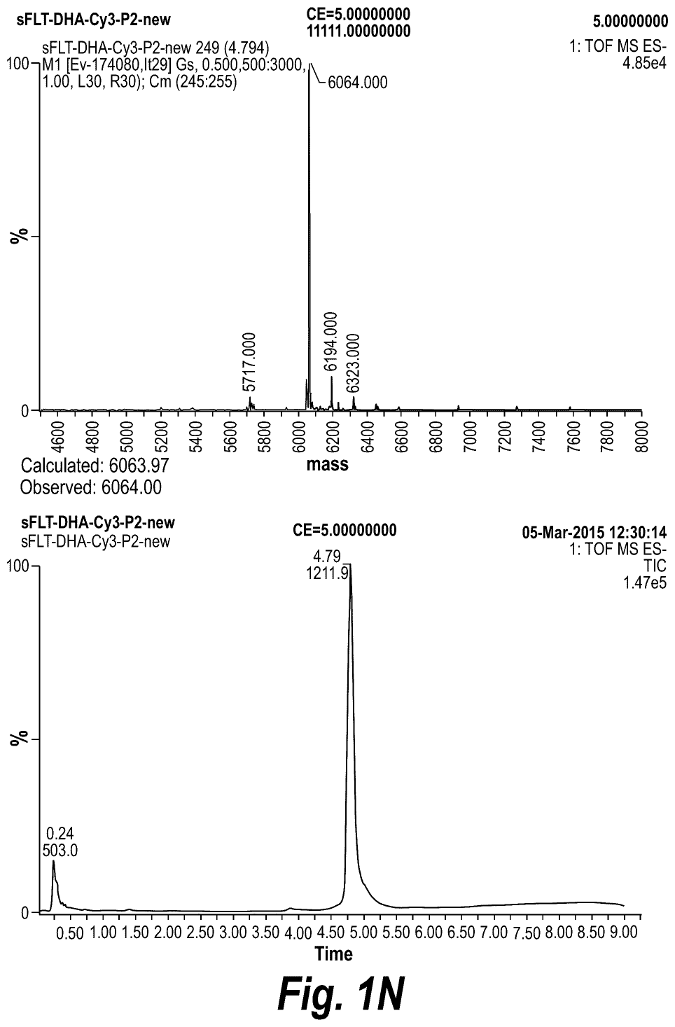

In one embodiment, an oxygen atom of L is bonded to the 3′ position of the second oligonucleotide by a phosphodiester for example, as shown in FIG. 1j.

### Variable X^{c}

In one embodiment, X^{c} has an affinity for low density lipoprotein and/or intermediate density lipoprotein. In a related embodiment, X^{c} is a saturated or unsaturated moiety having fewer than three double bonds.

In another embodiment, X^{c} has an affinity for high density lipoprotein. In a related embodiment, X^{c} is a polyunsaturated moiety having at three or more double bonds (e.g., having three, four, five, six, seven, eight, nine or ten double bonds). In a particular embodiment, X^{c} is a polyunsaturated moiety having three double bonds. In a particular embodiment, X^{c} is a polyunsaturated moiety having four double bonds. In a particular embodiment, X^{c} is a polyunsaturated moiety having five double bonds. In a particular embodiment, X^{c} is a polyunsaturated moiety having six double bonds.

In another embodiment, X^{c} is selected from the group consisting of fatty acids, steroids, secosteroids, lipids, gangliosides and nucleoside analogs, and endocannabinoids.

In another embodiment, X^{c} is a neuromodulatory lipid, e.g., an endocannabinoid. Non-limiting examples of endocannabinoids include: Anandamide, Arachidonoylethanolamine, 2-Arachidonyl glyceryl ether (noladin ether), 2-Arachidonyl glyceryl ether (noladin ether), 2-Arachidonoyl glycerol, and N-Arachidonoyl dopamine.

In another embodiment, X^{c} is an omega-3 fatty acid. Non-limiting examples of omega-3 fatty acids include: Hexadecatrienoic acid (HTA), Alpha-linolenic acid (ALA), Stearidonic acid (SDA), Eicosatrienoic acid (ETE), Eicosatetraenoic acid (ETA), Eicosapentaenoic acid (EPA, Timnodonic acid), Heneicosapentaenoic acid (HPA), Docosapentaenoic acid (DPA, Clupanodonic acid), Docosahexaenoic acid (DHA, Cervonic acid), Tetracosapentaenoic acid, and Tetracosahexaenoic acid (Nisinic acid).

In another embodiment, X^{c} is an omega-6 fatty acid. Non-limiting examples of omega-6 fatty acids include: Linoleic acid, Gamma-linolenic acid (GLA), Eicosadienoic acid, Dihomo-gamma-linolenic acid (DGLA), Arachidonic acid (AA), Docosadienoic acid, Adrenic acid, Docosapentaenoic acid (Osbond acid), Tetracosatetraenoic acid, and Tetracosapentaenoic acid.

In another embodiment, X^{c} is an omega-9 fatty acid. Non-limiting examples of omega-9 fatty acids include: Oleic acid, Eicosenoic acid, Mead acid, Erucic acid, and Nervonic acid.

In another embodiment, X^{c} is a conjugated linolenic acid. Non-limiting examples of conjugated linolenic acids include: α-Calendic acid, β-Calendic acid, Jacaric acid, α-Eleostearic acid, β-Eleostearic acid, Catalpic acid, and Punicic acid.

In another embodiment, X^{c} is a saturated fatty acid. Non-limiting examples of saturated fatty acids include: Caprylic acid, Capric acid, Docosanoic acid, Lauric acid, Myristic acid, Palmitic acid, Stearic acid, Arachidic acid, Behenic acid, Lignoceric acid, and Cerotic acid.

In another embodiment, X^{c} is an acid selected from the group consisting of: Rumelenic acid, α-Parinaric acid, β-Parinaric acid, Bosseopentaenoic acid, Pinolenic acid, and Podocarpic acid.

In another embodiment, X^{c} is selected from the group consisting of: docosanoic acid (DCA), docosahexaenoic acid (DHA), and eicosapentaenoic acid (EPA). In a particular embodiment, X^{c} is docosanoic acid (DCA). In another particular embodiment, X^{c} is DHA. In another particular embodiment, X^{c} is EPA.

In another embodiment, X^{c} is a secosteroid. In a particular embodiment, X^{c} is calciferol. In another embodiment, X^{c} is a steroid other than cholesterol.

In a particular embodiment, X^{c} is not cholesterol.

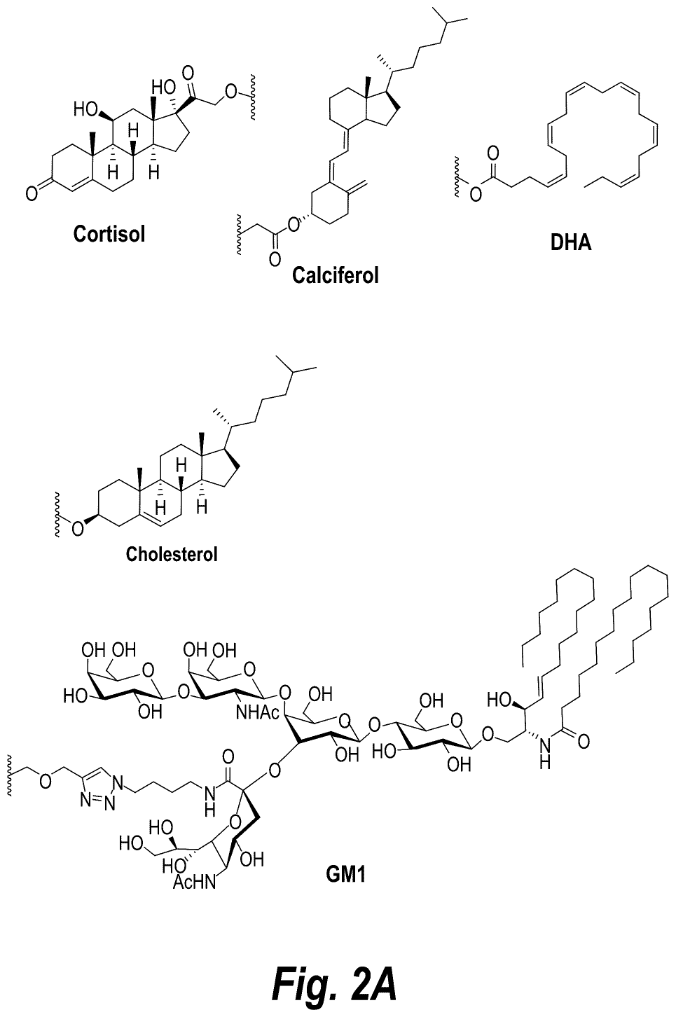

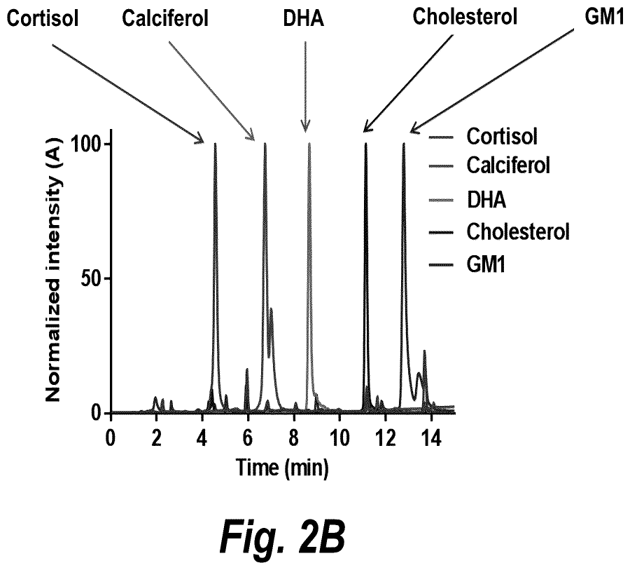

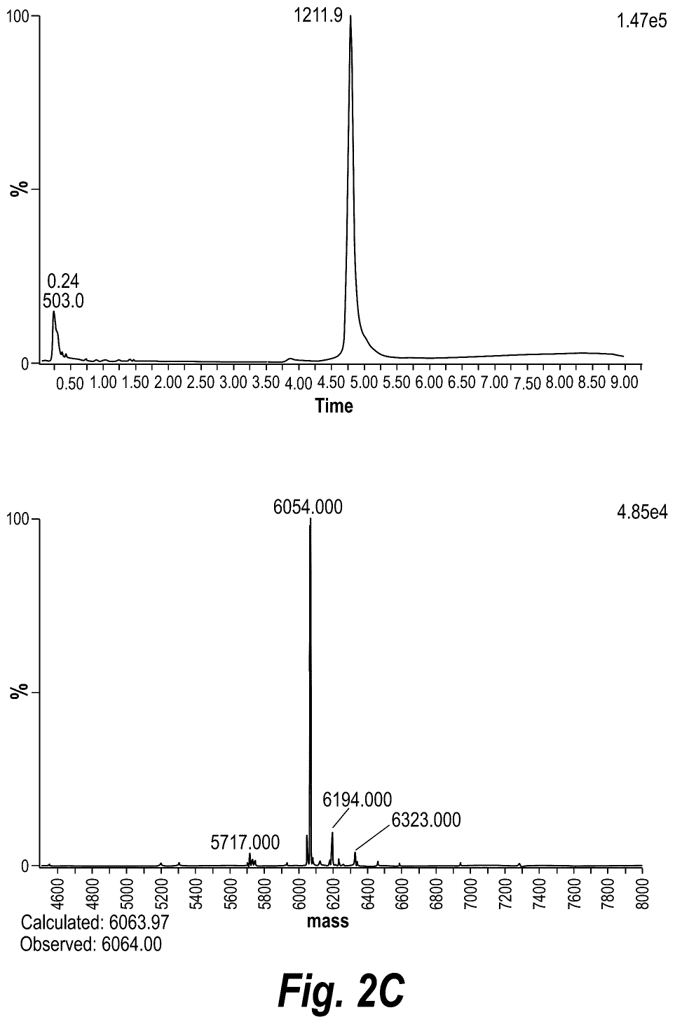

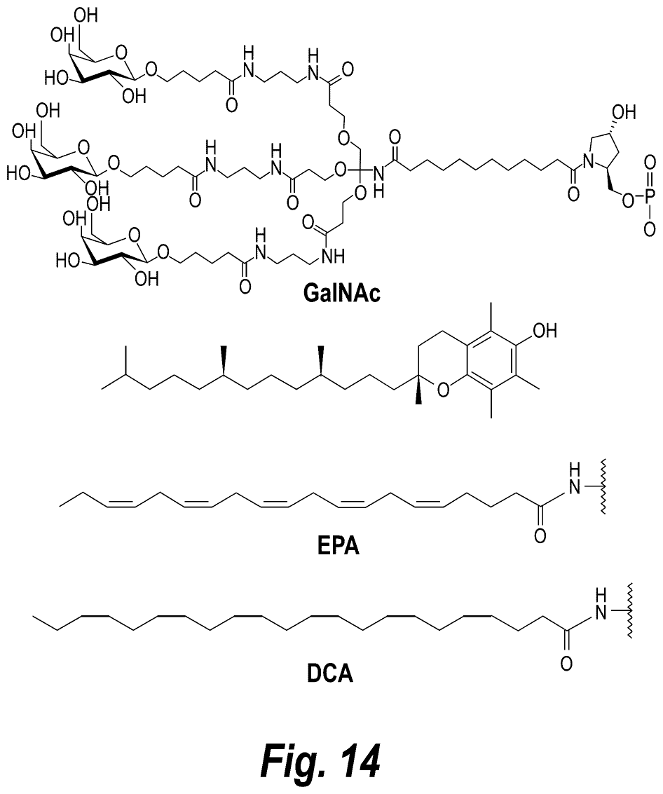

In another embodiment, X^{c} is an alkyl chain, a vitamin, a peptide, or a bioactive conjugate (including but not limited to: glycosphingolipids, polyunsaturated fatty acids, secosteroids, steroid hormones, sterol lipids. In other embodiments, the hydrophobic moiety comprises a moiety depicted in FIGS. 2a and 14.

In another embodiment of the oligonucleotide, X^{c} is characterized by a clogP value in a range selected from: −10 to −9, −9 to −8, −8 to −7, −7 to −6, −6 to −5, −5 to −4, −4 to −3, −3 to −2, −2 to −1, −1 to 0, 0 to 1, 1 to 2, 2 to 3, 3 to 4, 4 to 5, 5 to 6, 6 to 7, 7 to 8, 8 to 9, and 9 to 10.

### Variable Z^{c}

In one embodiment, Z^{c} is selected from the group consisting of Z^{c1}, Z^{c2}, z^{c3} and z^{c4}:

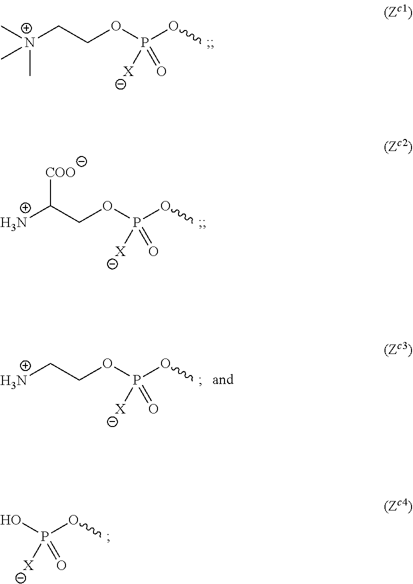

wherein X is O, S or BH₃.

In a particular embodiment, Z^{c} is Z^{c1}, In another particular embodiment, Z^{c} is not Z^{c1}.

In another embodiment, Z^{c} is selected from the group consisting of Z^{c2}, Z^{c3} and Z^{c4}. In a particular embodiment, Z^{c} is Z^{c2}, In a particular embodiment, Z^{c} is Z^{c3}, In a particular embodiment, Z^{c} is Z^{c4}, In a particular embodiment, X is O. In a particular embodiment, X is S. In a particular embodiment, X is BH₃.

### Proviso

In a particular embodiment of compound (1), when X^{c} is DHA, Z^{c} is not Z^{c1}. In another particular embodiment, when Z^{c} is Z^{c1}, X^{c} is not DHA.

### Variable O

In an embodiment, O comprises an oligonucleotide which may optionally be chemically modified. As used herein, an oligonucleotide refers to a short polymer of nucleotides and/or nucleotide analogs. The term “RNA analog” refers to an polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. Oligonucleotides may be linked with linkages which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Preferred RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA interference.

In certain embodiments, an oligonucleotide comprises a DNA polymer, an RNA polymer or a DNA/RNA polymer hybrid. In certain exemplary embodiments, an oligonucleotide comprises an RNA silencing agent or a gene silencing agent.

Exemplary RNA silencing agents include, but are not limited to, siRNAs, miRNAs, siRNA-like duplexes, antisense oligonucleotides, GAPMER molecules, and dual-function oligonucleotides as well as precursors thereof. In one embodiment, the RNA silencing agent is capable of inducing RNA interference. In another embodiment, the RNA silencing agent is capable of mediating translational repression.

An exemplary gene silencing agent is a CRISPR-Cas9-type system that utilizes a guide RNA (gRNA) oligonucleotide agnet.

In an embodiment, O comprises compound (I): an oligonucleotide of at least 16 contiguous nucleotides, said oligonucleotide having a 5′ end, a 3′ end and complementarity to a target. In a particular embodiment, the target is mammalian or viral mRNA. In another particular embodiment, the target is an intronic region of said mRNA. In one embodiment, the oligonucleotide has sufficient complementarity to the target to hybridize. In certain embodiments, the complementarity is >95%, >90%, >85%, >80%, >75%, >70%, >65%, >60%, >55% or >50%. In one embodiment, compound (I) has perfect complementarity to the target.

In an embodiment, compound (I) comprises one or more chemically-modified nucleotides. In a particular embodiment, compound (I) comprises alternating 2′-methoxy-nucleotides and 2′-fluoro-nucleotides. In another particular embodiment, one or more nucleotides of compound (I) are connected to adjacent nucleotides via phosphorothioate linkages.

In an embodiment, the 5′ terminus of compound (I) comprises a moiety selected from the group of:

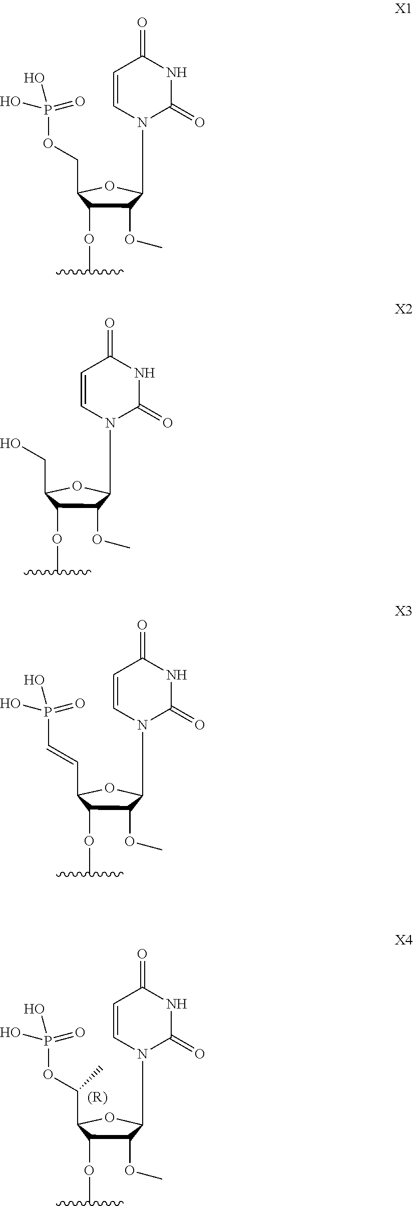

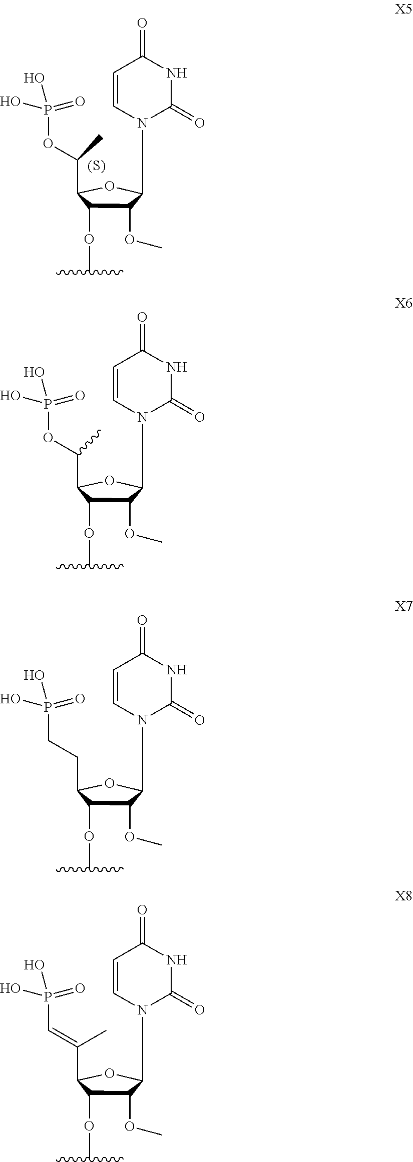

In another embodiment, O comprises compound (II): an oligonucleotide of at least 15 contiguous nucleotides, said oligonucleotide having a 5′ end, a 3′ end, and homology with a target, wherein the oligonucleotide is conjugated at the 3′ end to -L(X^{c})(Z^{c}), defined above.

In an embodiment, compound (II) has complete homology with the target. In a particular embodiment, the target is mammalian or viral mRNA. In another particular embodiment, the target is an intronic region of said mRNA.

In an embodiment, compound (II) comprises one or more chemically-modified nucleotides. In a particular embodiment, compound (II) comprises alternating 2′-methoxy-nucleotides and 2′-fluoro-nucleotides. In another particular embodiment, the nucleotides at positions 1 and 2 from the 3′ end of compound (II) are connected to adjacent nucleotides via phosphorothioate linkages. In yet another particular embodiment, the nucleotides at positions 1 and 2 from the 3′ end of compound (II) and the nucleotides at positions 1 and 2 from the 5′ end of compound (II) are connected to adjacent nucleotides via phosphorothioate linkages.

In an embodiment, O is a double-stranded nucleic acid comprising a first oligonucleotide and a second oligonucleotide, wherein:
(1) the first oligonucleotide is compound (I), or any one of the previous embodiments thereof;
(2) the second oligonucleotide is compound (II), or any one of the previous embodiments thereof; and
(3) a portion of the first oligonucleotide is complementary to a portion of the second oligonucleotide.

In one embodiment of O, the first oligonucleotide comprises at least 16 contiguous nucleotides, a 5′ end, a 3′ end, and has complementarity to a target, wherein:
(1) the first oligonucleotide comprises alternating 2′-methoxy-nucleotides and 2′-fluoro-nucleotides;
(2) the nucleotides at positions 2 and 14 from the 5′ end are not 2′-methoxy-nucleotides;
(3) the nucleotides are connected via phosphodiester or phosphorothioate linkages; and
(4) the nucleotides at positions 1-6 from the 3′ end, or positions 1-7 from the 3′ end, are connected to adjacent nucleotides via phosphorothioate linkages.

In a particular embodiment of the double-stranded nucleic acid, the first oligonucleotide has perfect complementarity to the target.

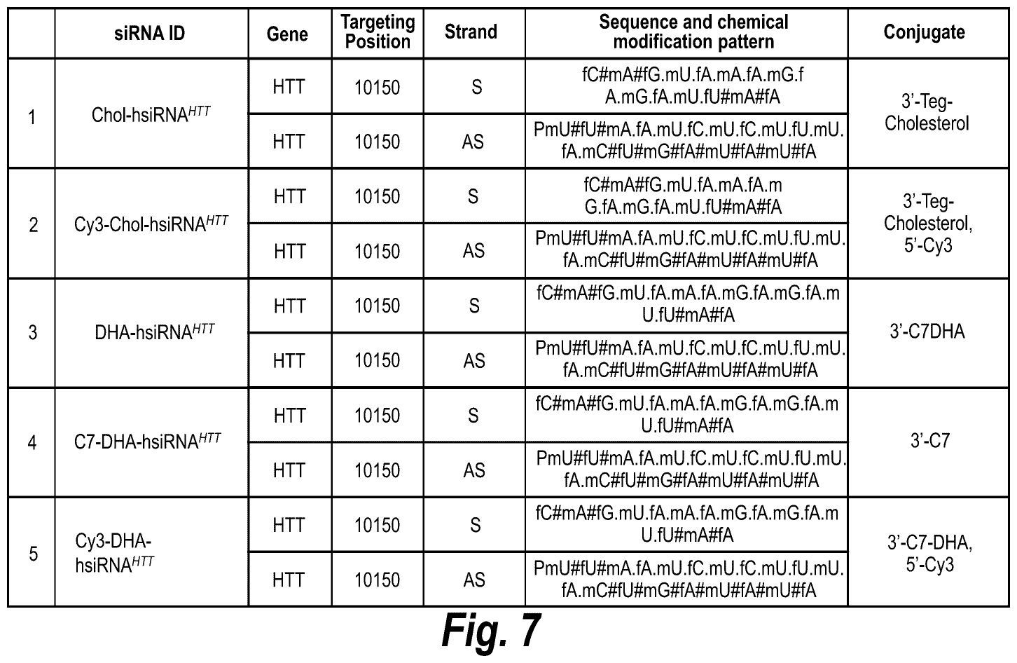

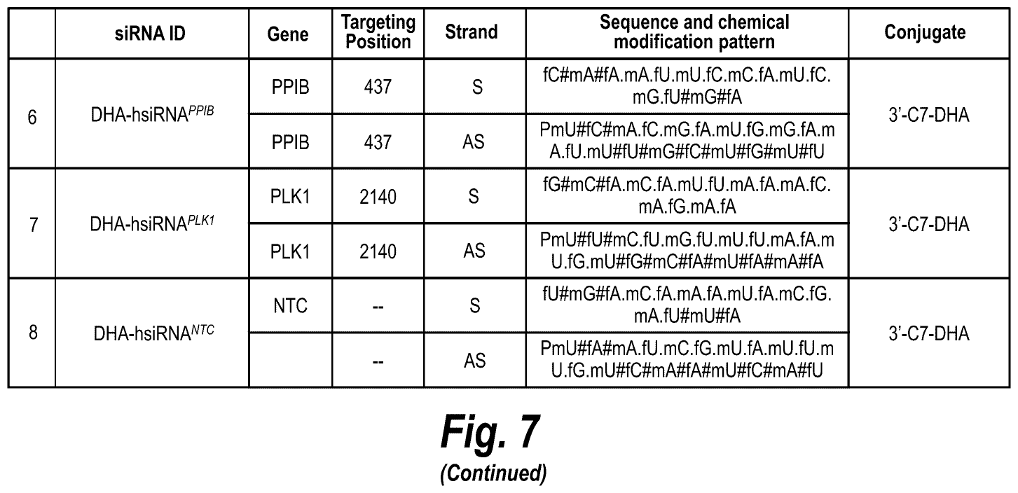

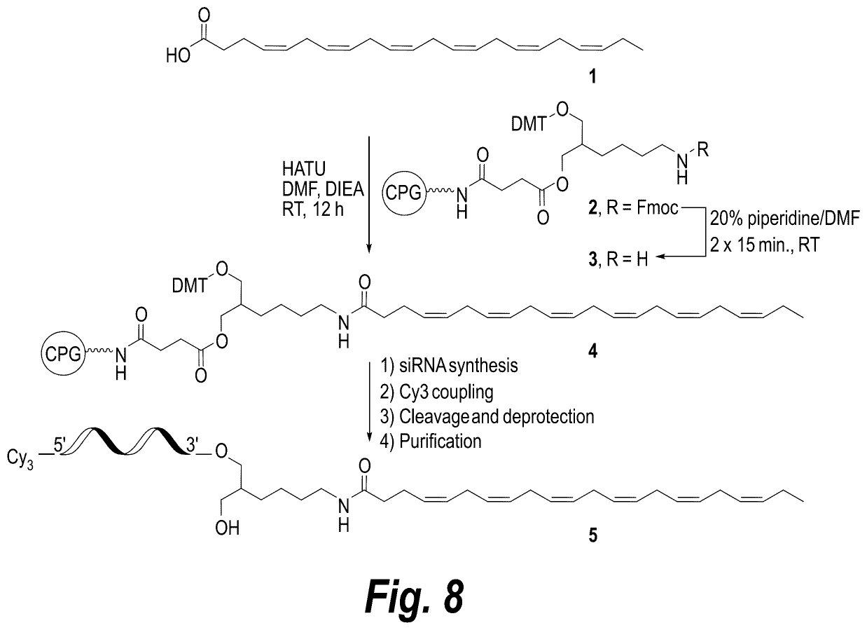

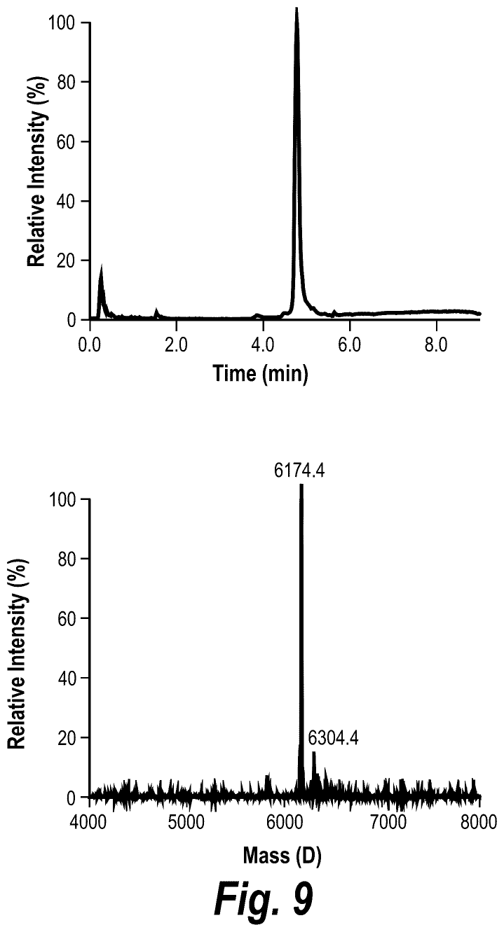

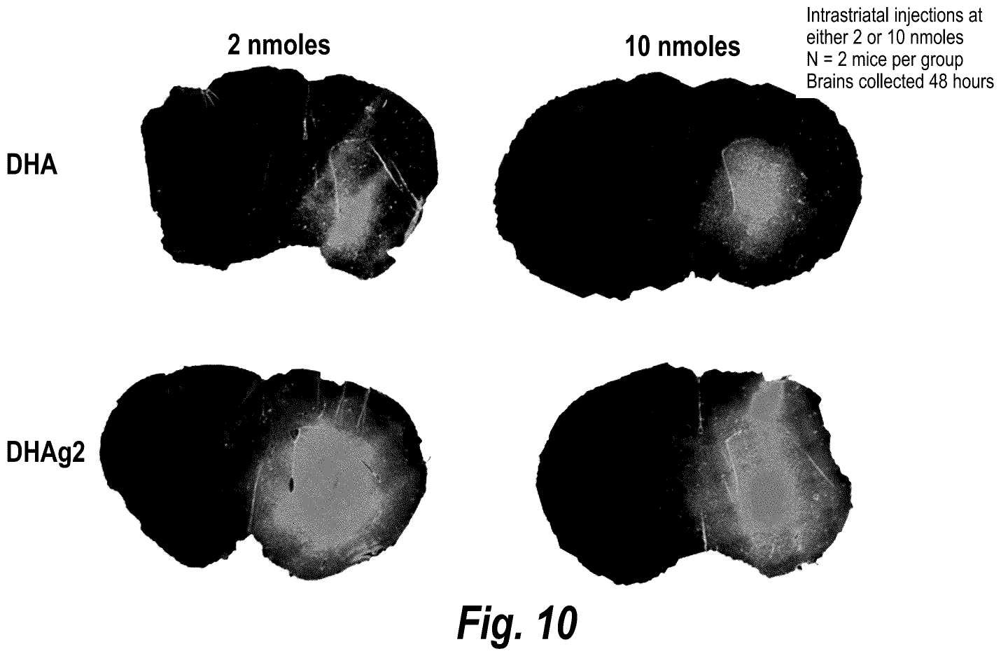

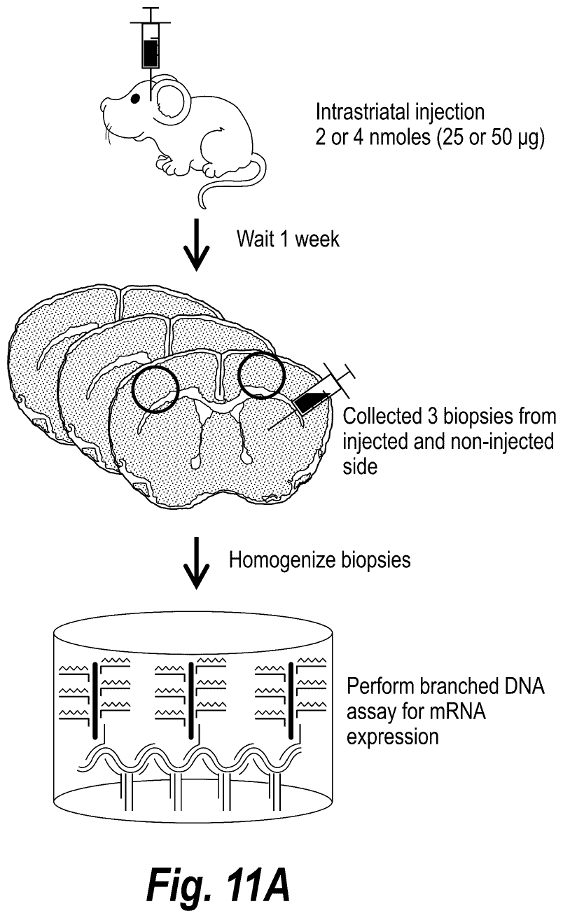

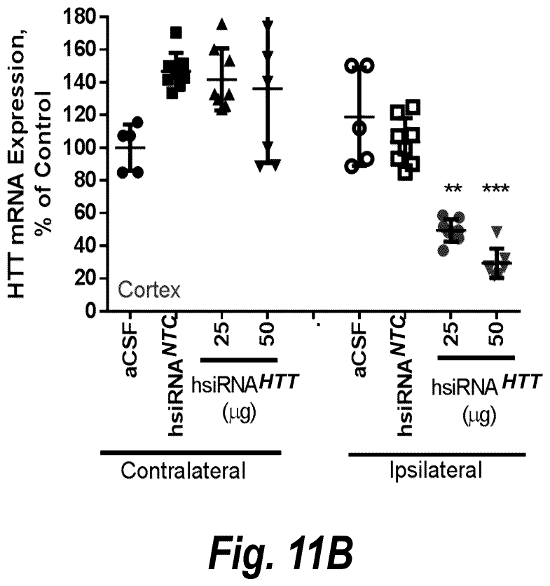

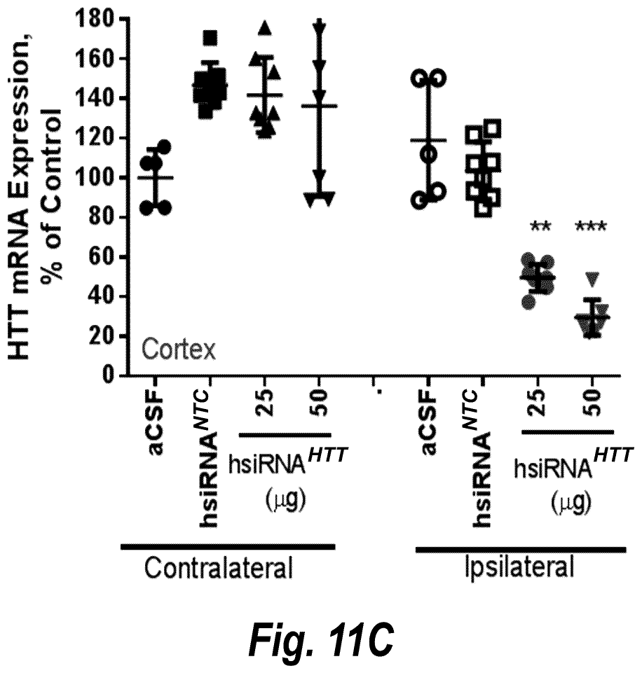

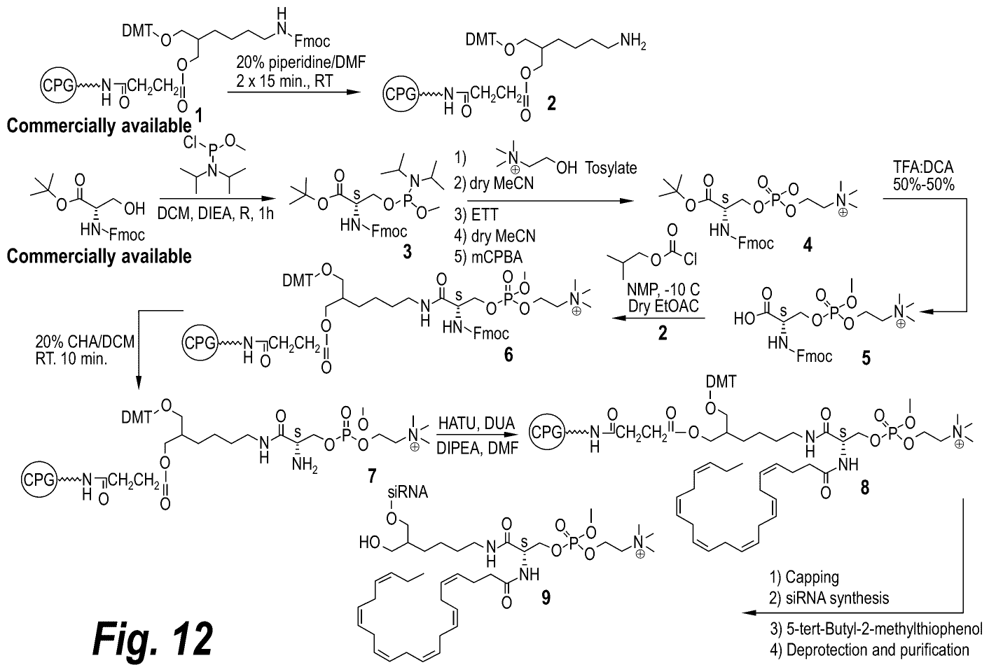

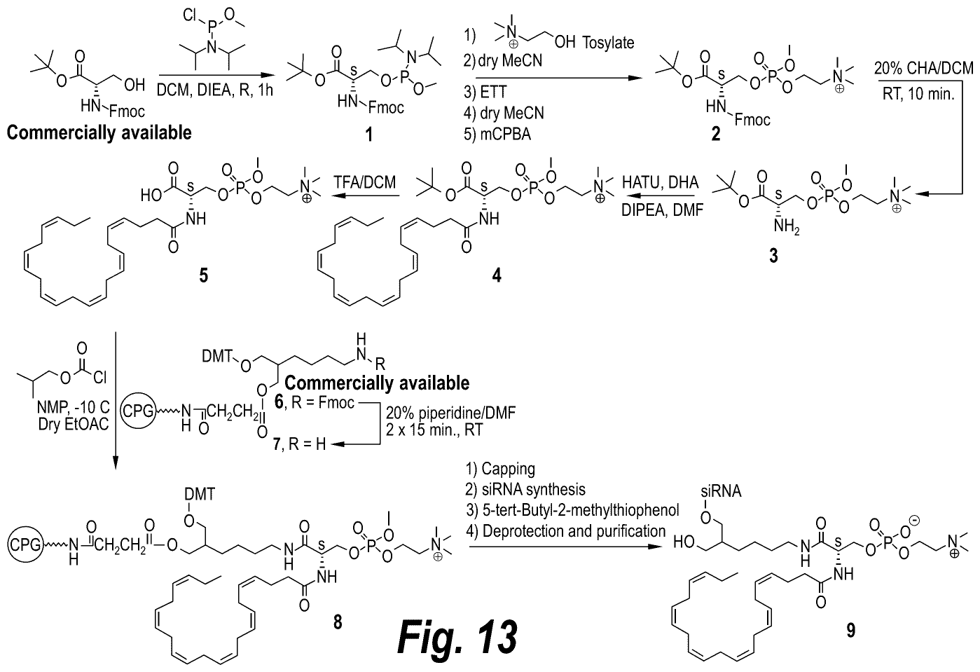

In another particular embodiment of the double-stranded nucleic acid, the sequences of the first and second oligonucleotides are selected from FIG. 7.

### Advanced Stabilization Pattern

In one embodiment, compound (I) has the structure of Formula (Ia):

X(—K—B—K-A)ⱼ(—S—B—S-A)ᵣ(—S—B)ₜ—OR (Ia)
wherein:
X is selected from the group consisting of X1, X2, X3, X4, X5, X6, X7 and X8, defined above.
A, for each occurrence, independently is a 2′-methoxy-ribonucleotide;
B, for each occurrence, independently is a 2′-fluoro-ribonucleotide;
K, for each occurrence independently is a phosphodiester or phosphorothioate linker;
S is a phosphorothioate linker;
R is selected from hydrogen and a capping group (e.g., an acyl group such as acetyl);
j is 4, 5, 6 or 7;
r is 2 or 3; and
t is 0 or 1.

In one embodiment, the oligonucleotide of Formula (Ia) has the structure of Formula (Ib):

X-A(—S—B—S-A)ₘ(—P—B—P-A)ₙ(—P—B—S-A)_{q}(—S—B—S-A)ᵣ(—S—B)ₜ—OR (Ib)
wherein:
X is as defined above;
A, for each occurrence, independently is a 2′-methoxy-ribonucleotide;
B, for each occurrence, independently is a 2′-fluoro-ribonucleotide;
S is a phosphorothioate linker;
P is a phosphodiester linker;
R is as defined above;
m is 0 or 1; n is 4, 5 or 6; q is 0 or 1; r is 2 or 3; and t is 0 or 1.

In a particular embodiment, R is hydrogen. In another particular embodiment, X is X1. In still another particular embodiment, X is X3.

In another embodiment, O is a double-stranded, chemically-modified nucleic acid comprising a first oligonucleotide and a second oligonucleotide, wherein:
(1) the first oligonucleotide is selected from the oligonucleotides of Formulas (I) and (Ia);
(2) a portion of the first oligonucleotide is complementary to a portion of the second oligonucleotide; and
(3) the second oligonucleotide is selected from the oligonucleotides of Formulas (II) and (IIa):

C-L-B(—S-A-S—B)_{m′}(—P-A-P—B)_{n′}(—P-A-S—B)_{q′}—S-A)_{r′}(—S—B)_{t′}—OR (IIa)
wherein:
   C-L is:

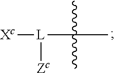

wherein
   L; Z^{c}; A; B; S; P are defined above
m′ is 0 or 1; n′ is 4, 5 or 6; q′ is 0 or 1; r′ is 0 or 1; and t′ is 0 or 1.

In one embodiment of compound (1):
(1) the first oligonucleotide comprises alternating 2′-methoxy-ribonucleotides and 2′-fluoro-ribonucleotides, wherein each nucleotide is a 2′-methoxy-ribonucleotide or a 2′-fluoro-ribonucleotide; and the nucleotides at positions 2 and 14 from the 5′ end of the first oligonucleotide are not 2′-methoxy-ribonucleotides;
(2) the second oligonucleotide comprises alternating 2′-methoxy-ribonucleotides and 2′-fluoro-ribonucleotides, wherein each nucleotide is a 2′-methoxy-ribonucleotide or a 2′-fluoro-ribonucleotide; and the nucleotides at positions 2 and 14 from the 5′ end of the second oligonucleotide are 2′-methoxy-ribonucleotides;
(3) the nucleotides of the first oligonucleotide are connected to adjacent nucleotides via phosphodiester or phosphorothioate linkages, wherein the nucleotides at positions 1-6 from the 3′ end, or positions 1-7 from the 3′ end are connected to adjacent nucleotides via phosphorothioate linkages; and
(4) the nucleotides of the second oligonucleotide are connected to adjacent nucleotides via phosphodiester or phosphorothioate linkages, wherein the nucleotides at positions 1 and 2 from the 3′ end are connected to adjacent nucleotides via phosphorothioate linkages.

In one embodiment of O, the first oligonucleotide has 3-7 more ribonucleotides than the second oligonucleotide.

In one embodiment, O comprises 11-16 base pair duplexes, wherein the nucleotides of each base pair duplex have different chemical modifications (e.g., one nucleotide has a 2′-fluoro modification and the other nucleotide has a 2′-methoxy).

In one embodiment of O, the first oligonucleotide has 3-7 more ribonucleotides than the second oligonucleotide. In another embodiment, each R is hydrogen.

In one embodiment, the first oligonucleotide is the antisense strand and the second oligonucleotide is the sense strand.

### Pharmaceutical Compositions and Methods of Administration

In one aspect, provided herein is a pharmaceutical composition comprising a therapeutically effective amount of one or more compound, oligonucleotide, or nucleic acid as described herein, and a pharmaceutically acceptable carrier. In one embodiment, the pharmaceutical composition comprises one or more double-stranded, chemically-modified nucleic acid as described herein, and a pharmaceutically acceptable carrier. In a particular embodiment, the pharmaceutical composition comprises one double-stranded, chemically-modified nucleic acid as described herein, and a pharmaceutically acceptable carrier. In another particular embodiment, the pharmaceutical composition comprises two double-stranded, chemically-modified nucleic acids as described herein, and a pharmaceutically acceptable carrier.

A pharmaceutical composition of the invention is formulated to be compatible with its intended route of administration. Examples of routes of administration include parenteral, e.g., intravenous (IV), intradermal, subcutaneous (SC or SQ), intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

### Methods of Treatment

In one aspect, provided herein is a method for delivering (e.g., selectively delivering) an oligonucleotide conjugate described herein to a target in an organ or a tissue in a subject, comprising administering said oligonucleotide conjugate to the subject. In certain embodiments, the oligonucleotide conjugate may have a selective affinity for a serum lipoprotein.

As used herein, a “target” refers to a particular nucleic acid sequence (e.g., a gene, an mRNA, a miRNA or the like) that an oligonucleotide conjugate of the invention binds to and/or otherwise effects the expression of. In certain embodiments, the target is expressed in a particular tissue, organ or tissue type. In other embodiments, a target is associated with a particular disease or disorder in a subject. Exemplary diseases and disorders are described herein.

One of ordinary skill in the art would readily appreciate that a nucleic acid sequence (e.g., an mRNA) associated with a particular tissue, organ, disease or disorder may be targeted using an oligonucleotide (e.g., an siRNA) conjugated to a hydrophobic compound described herein using the guidance provided herein and the knowledge in the art.

For example, an mRNA that is associated with a neurodegenerative disorder such as Huntington's disease (HD) and/or expressed in a particular neural tissue (e.g., brain or CNS) could be targeted for downregulation using an siRNA conjugated to a hydrophobic compound as described herein that is sufficiently complementary to a variety of well-known HTT mRNA sequences to downregulate their expression, thus treating or lessening one or more symptoms of HD in an subject. Examples of HD mRNA sequences such as, e.g., particular known HTT mRNA 3′ untranslated region (UTR) sequences, are described in WO 2016/161374, incorporated herein by reference in its entirety for all purposes.

Similarly, one of ordinary skill in the art could readily target an mRNA associated with an angiogenic disorder such as pre-eclampsia (PE) and/or expressed in a particular tissue (e.g., placenta) by targeting a variety of well-known PE soluble FLT (“sFLT”) mRNAs such as sFLT1-i13 short isoform, sFLT1-i13 long isoform, sFLT1415a or the like for downregulation using an siRNA conjugated to a hydrophobic compound as described herein that is sufficiently complementary to the sFLT target, thus treating or otherwise reducing one or more symptoms of PE. Suitable PE mRNA sequences for targeting are described in WO 2016/161378, incorporated herein by reference in its entirety for all purposes.

In one embodiment, the organ is the kidney and the compound has a selective affinity for high density lipoprotein versus low density lipoprotein and/or high density lipoprotein. In a particular embodiment, the organ is the kidneys and X^{c} is a polyunsaturated moiety having at three or more double bonds (e.g., DHA).

In another embodiment, the organ is the liver and the compound has a selective affinity for low density lipoprotein and/or high density lipoprotein versus high density lipoprotein. In a particular embodiment, the organ is the liver and X^{c} is a moiety that is saturated or has fewer than three double bonds.

In another embodiment, the organ is the brain and the compound has a selective affinity for high density lipoprotein versus low density lipoprotein and/or high density lipoprotein. In a particular embodiment, the organ is the brain and X^{c} is a polyunsaturated moiety having three or more double bonds (e.g., DHA).

In another embodiment, the organ is the placenta and the compound has a selective affinity for high density lipoprotein versus low density lipoprotein and/or high density lipoprotein. In a particular embodiment, the organ is the placenta and X^{c} is PC-DCA or DCA.

In another embodiment, the organ is the epidermis and the compound has a selective affinity for high density lipoprotein versus low density lipoprotein and/or high density lipoprotein. In a particular embodiment, the organ is the epidermis and X^{c} is a polyunsaturated moiety having three or more double bonds (e.g., EPA).

In another aspect, provided herein is a method for delivering (e.g., selectively delivering) a compound of formula (1), or a disclosed embodiment thereof, to the kidneys of a patient, comprising administering said compound to the patient intravenously, wherein X^{c} is a polyunsaturated moiety having three or more double bonds (e.g., DHA).

In another aspect, provided herein is a method for treating a disease or disorder of the kidneys in a patient in need of such treatment, comprising administering to the patient a compound of formula (1), or a disclosed embodiment thereof. Non-limiting examples of such disease or disorders include: Abderhalden-Kaufmann—Lignac syndrome; Acute kidney injury; Acute proliferative glomerulonephritis; Adenine phosphoribosyltransferase deficiency; Alport syndrome; Analgesic nephropathy; Autosomal dominant polycystic kidney disease; Autosomal recessive polycystic kidney disease; Benign nephrosclerosis; Bright's disease; Cardiorenal syndrome; CFHR5 nephropathy; Chronic kidney disease; Chronic kidney disease-mineral and bone disorder; Congenital nephrotic syndrome; Conorenal syndrome; Contrast-induced nephropathy; Cystic kidney disease; Danubian endemic familial nephropathy; Dent's disease; Diabetic nephropathy; Diffuse proliferative nephritis; Distal renal tubular acidosis; Diuresis; EAST syndrome; Epithelial-mesenchymal transition; Fanconi syndrome; Fechtner syndrome; Focal proliferative nephritis; Focal segmental glomerulosclerosis; Fraley syndrome; Galloway Mowat syndrome; Gitelman syndrome; Glomerulocystic kidney disease; Glomerulopathy; Glomerulosclerosis; Goldblatt kidney; Goodpasture syndrome; High anion gap metabolic acidosis; HIV-associated nephrapathy; Horseshoe kidney; Hydronephrosis; Hypertensive nephropathy; IgA nephropathy; Interstitial nephritis; Juvenile nephronophthisis; Kidney cancer; Lightwood-Albright syndrome; Lupus nephritis; Malarial nephropathy; Medullary cystic kidney disease; Medullary sponge kidney; Membranous glomerulonephritis; Mesoamerican nephropathy; Milk-alkali syndrome; Minimal mesangial glomerulonephritis; Multicystic dysplastic kidney; Nephritis; Nephrocalcinosis; Nephrogenic diabetes insipidus; Nephromegaly; Nephroptosis; Nephrosis; Nephrotic syndrome; Nutcracker syndrome; Papillorenal syndrome; Phosphate nephropathy; Polycystic kidney disease; Primary hyperoxaluria; Proximal renal tubular acidosis; Pyelonephritis; Pyonephrosis; Rapidly progressive glomerulonephritis; Renal agenesis; Renal angina; Renal artery stenosis; Renal cyst; Renal ischemia; Renal osteodystrophy; Renal papillary necrosis; Renal tubular acidosis; Renal vein thrombosis; Reninoma; Serpentine fibula-polycystic kidney syndrome; Shunt nephritis; Sickle cell nephropathy; Thin basement membrane disease; Transplant glomerulopathy; Tubulointerstitial nephritis and uveitis; Tubulopathy; Uremia and Wunderlich syndrome.

In another aspect, provided herein is a method for delivering (e.g., selectively delivering) such treatment, comprising administering to the patient a compound disclosed herein to the liver of a patient, comprising administering said compound to the patient intravenously, wherein X^{c} is a moiety that is saturated or has fewer than three double bonds.

In another aspect, provided herein is a method for treating a disease or disorder of the brain in a patient in need of such treatment, comprising administering to the patient a compound of formula (1), or a disclosed embodiment thereof, Non-limiting examples of such disease or disorders include: Acute Disseminated Encephalomyelitis, Agnosia, Alpers' Disease, Angelman Syndrome, Asperger Syndrome, Alzheimer's Disease, Amyotrophic Lateral Sclerosis, Aneurysm, Attention Deficit Hyperactivity Disorder, Autism, Bell's Palsy, Batten Disease, Brain Cancer, Canavan Disease, Concussion, Coma, Cerebral Hypoxia, Cerebral Palsy, Creutzfeldt-Jakob Disease, Dementia, Dravet Syndrome, Dyslexia, Epilepsy, Encephalitis, Farber's Disease, Febrile Seizures, Friedreich's Ataxia, Gaucher Disease, Huntinton's Disease, Hypersomnia, Migraine, Multiple Sclerosis, Narcolepsy, Parkinson's Disease, Stroke, and Traumatic Brain Injury, Tremor, and Wallenberg's Syndrome.

In another aspect, provided herein is a method for treating a disease or disorder of the epidermis in a patient in need of such treatment, comprising administering to the patient a compound of formula (1), or a disclosed embodiment thereof, Non-limiting examples of such disease or disorders include: Ichthyosis, Ectodermal Dysplasia, Psoriasis, Eczema, Darier's Disease, Infantile acropustulosis, Acrokeratoelastoidosis, Pityriasis rubra pilaris, Glucagonoma Syndrome, Acrodermatitis enteropathica, Porokeratosis, Acne, Vitiligo, Skin Cancer, Grover's Disease, Alopecia, Dermatitis, Leiner's Disease, Xeroderma pigmentosum, Toxic Epidermal Necrolysis, Seborrheic Keratoses, Uticaria, Erythema Multiforme, Pemphigus Vulgaris, Bullous Pemphigoid, Scleroderma, and Lupus Erythematosus.

In another aspect, provided herein is a method for treating a disease or disorder of the liver in a patient in need of such treatment, comprising administering to the patient a compound of formula (1), or a disclosed embodiment thereof. Non-limiting examples of such disease or disorders include: liver disease; acute fatty liver of pregnancy; acute liver failure; alcoholic liver disease; alpha-1 antitrypsin deficiency; alveolar hydatid disease; bacillary peliosis; Budd-Chiari syndrome; liver cancer; chronic liver disease; cirrhosis; congenital hepatic fibrosis; congestive hepatopathy; epithelial-mesenchymal transition; fatty liver; fibrolamellar hepatocellular carcinoma; focal fatty liver; gastric antral vascular extasia; hepatic encephalopathy; hepatolithiasis; hepatopulmonary syndrome; hapatorenal syndrome; hepatosplenomegaly; Laennec's cirrhosis; liver abscess; liver failure; Lyngstadaas syndrome; non-alcoholic fatty liver disease; non-cirrhotic portal fibrosis; non-alcoholic fatty liver disease; non-cirrhotic portal fibrosis; non-alcoholic fatty liver disease; pediatric end-stage liver disease; peliosis hepatis; polycystic liver disease; primary biliary cirrhosis; progressive familial intrahepatic cholestasis; steatohepatitis; viral hepatitis; Wilson's diease; Zahn infarct; and Zieve's syndrome.

In certain aspects, the particular tissue or organ to be targeted is selected from the group consisting of thymus, bladder, intestine, skin, bone marrow, placenta, adipose tissue, muscle, spleen, pancreas, lung, fallopian tube, adrenal gland, heart, liver and kidney.

Diseases and conditions related to the thymus include but are not limited to myasthenia gravis (MG), pure red cell aplasia (PRCA), hypogammaglobulinemia and cancer.

Diseases and conditions related to the bladder include but are not limited to flaccid (hypotonic) neurogenic bladder, spastic bladder, a mix of flaccid and spastic bladder (“mixed type”), overflow incontinence, overflow dribbling, urinary tract infections, urinary calculi, cystitis, interstitial cystitis, bladder rupture, bladder obstruction (tamponade), paruresis, cystocele, bladder fistula, bladder stones, bladder exstrophy, bladder diverticulum and cancer.

Diseases and disorders related to the intestine (e.g., large and/or small intestine) include but are not limited to celiac disease, Crohn's disease, irritable bowel syndrome, ulcer (e.g., peptic ulcer), intestinal dysmobility, intestinal pseudo-obstruction, short bowel syndrome, intestinal malrotation, Meckel's diverticulum, superior mesenteric artery syndrome, necrotizing enterocolitis, duodenal artesia, enteritis, small bowel bacterial overgrowth, Yersinia enterocolitica infection, Yersinia pseudotuberculosis infection and cancer.

Diseases and disorders related to bone marrow include but are not limited to leukemia, lymphoma, aplastic anemia, myeloproliferative disorders, myelodysplastic syndromes and other cancers.

Diseases and disorders of the placenta include but are not limited to abruptio placentae, placenta accretia, placenta increta, placenta percreta, chorioamnionitis, intervillitis, TORCH infections (e.g., CMV placentitis), chronic deciduitis, circumvallate placenta, placental villous immaturity, placenta previa, vasa previa, fetal thrombic vasculopathy, hypertrophic decidual vasculopathy, chorangiosis, chorangioma, placental infarction, hydatidiform mole, choriocarcinoma and placental cancer.

Diseases and conditions related to adipose tissue include but are not limited to obesity, diabetes, insulin resistance, lipodystrophies, Dercum's disease, adipose tissue neoplasm, general adipose tissue inflammation, cardiovascular disease, hypertension and stroke, hypercholesterolemia, hypertriglyceridemia, arthritis, asthma and cancer.

Lipodystrophies include Berardinelli-Seip syndrome, Barraquer-Simons syndrome, Lawrence-Seip syndrome, Centrifugal lipodystrophy, Ferreira-Marques lipoatrophia, Familial Partial lipodystrophy, Dunnigan syndrome, Localized lipodystrophy, metabolic syndrome, and HIV-related lipodystrophy.

Adipose tissue acts as an endocrine organ, secreting proteinaceous hormones called adipokines. The action of these adipokines effects numerous cellular functions. They regulate food intake, insulin sensitivity, fatty acid oxidation rates, serum fatty acid and glucose levels, and cellular growth and differentiation. Exemplary adipokines include but are not limited to leptin, ghrelin, adiponectin, apelin, chemerin, interleukin-6 (IL-6), monocyte chemotactic protein-1 (MCP-1), plasminogen activator inhibitor-1 (PAI-1), retinol binding protein (RBP4), tumor necrosis factor-alpha (TNFα), visfatin, omentin, vaspin, and progranulin. In a further embodiment, the oligonucleotide conjugates of the present invention can be used to modulate (increase or decrease) the expression of adipokines.

Diseases and conditions related to muscle tissue include but are not limited to myositis, myotonia congenita, paramyotonia congenita, periodic paralyses, central core disease/malignant hyperthermia susceptibility, nemaline myopathy, centronuclear myopathies, sarcopenia, mitochondrial myopathies, hypotonia, muscular dystrophy, dermatomyositis, cerebral palsy, compaftment syndrome, myasthenia gravis, amyotrophic lateral sclerosis (ALS), rhabdomyolysis, polymyositis, fibromyalgia, myofascial pain syndrome, muscle cramps, muscle sprains and strains, and tendonitis.

Diseases and conditions related to the spleen include but are not limited to splenomegaly, splenic disease, Gaucher's disease, asplenia, splenic infarction, spherocytosis, wandering spleen, splenic tumors, infectious mononucleosis, splenic injury, hyaloserositis, and anemias.

Diseases and conditions related to the pancreas include but are not limited to pancreatitis, pancreatic cancer, cystic fibrosis, pseudocyst, exocrine pancreatic insufficiency, diabetes, gastrointestinal diseases, pancreas divisum, steatorrhea, and sphincter of Oddi dysfunction.

Much like adipose tissue, the pancreas is a major endocrine organ. Exemplary secreted pancreatic hormones include but are not limited to glucagon, insulin, pancreatic polypeptide, preproinsulin, proglucagon, somatostatin, vasoactive intestinal peptide, growth hormone releasing hormone, gastrin, ghrelin, amylin, secretin, and cholecystokinin. In a further embodiment, the oligonucleotide conjugates of the present invention can be used to modulate (increase or decrease) the expression of pancreatic endocrine hormones.

In addition to pancreatic endocrine function, the pancreas has exocrine functions, i.e., the production of enzymes involved in digestion. Exemplary secreted pancreatic exocrine enzymes include but are not limited to trypsin, chymotrypsin, lipases, amylases, nucleases, gelatinase, and elastase. In a further embodiment, the oligonucleotide conjugates of the present invention can be used to modulate (increase or decrease) the expression of pancreatic exocrine enzymes. Diseases and disorders of the lung include but are not limited to chronic obstructive pulmonary disease, asthma, chronic bronchitis, acute bronchitis, emphysema, cystic fibrosis, pneumonia, tuberculosis, pulmonary edema, lung cancer, acute respiratory distress syndrome, pneumoconiosis, interstitial lung disease (e.g., sarcoidosis, idopathic pulmonary fibrosis, autoimmune disease), pulmonary embolism, pulmonary hypertension, pleural effusion, pneumothorax, mesothelioma and obesity hypoventilation syndrome.

Diseases and conditions related to the fallopian tube include but are not limited to salpingitis, endosalpingiosis, tubal torsion, paratubal cyst, endometriosis, fallopian tube cancers, infertility, fallopian tube obstruction, and adhesions.

Diseases and conditions related to the adrenal gland include but are not limited to Addison's disease, adrenal tumors, adrenal insufficiency, adrenal hyperplasia, primary aldosteronism, hyperaldosteronism, hypoaldosteronism, adrenal crisis, Cushing's disease, adrenocortical hyperfunction, adrenoleukodystrophy, adrenal fatigue, and adrenal incidentaloma.

The adrenal gland is another major endocrine organ. Exemplary secreted adrenal hormones include but are not limited to aldosterone, cortisol, adrenaline, noradrenaline, epinephrine, dehydroepiandrosterone, testosterone, and estrogen. In a further embodiment, the oligonucleotide conjugates of the present invention can be used to modulate (increase or decrease) the expression of adrenal gland hormones.

Diseases and conditions related to the heart include but are not limited to atherosclerosis, coronary artery disease, myocarditis, endocarditis, pericarditis, rheumatic heart disease, hypertensive heart disease, aneurysm, angina, myocardial infarction, cardiomyopathy, valvular heart disease, congential heart disease, heart failure, arrhythmia, cardiac arrest, congestive heart failure, atrial fibrillation, Brugada syndrome, tachycardia, Catecholaminergic Polymorphic Ventricular Tachycardia (CPVT), long QT syndrome, Progressive Cardiac Conduction Defect (PCCD), stroke, Peripheral Artery Disease (PAD), thromboembolisms, high blood pressure, heart murmurs, Kawasaki disease, DiGeorge syndrome, pre-eclampsia, and cardiac tumors.

“Treatment,” or “treating,” as used herein, is defined as the application or administration of a therapeutic agent (e.g., a RNA agent or vector or transgene encoding same) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the invention provides a method for preventing in a subject, a disease or disorder as described above, by administering to the subject a therapeutic agent (e.g., an RNAi agent or vector or transgene encoding same). Subjects at risk for the disease can be identified by, for example, any or a combination of diagnostic or prognostic assays as described herein. Administration of a prophylactic agent can occur prior to the manifestation of symptoms characteristic of the disease or disorder, such that the disease or disorder is prevented or, alternatively, delayed in its progression.

Another aspect of the invention pertains to methods treating subjects therapeutically, i.e., alter onset of symptoms of the disease or disorder. In an exemplary embodiment, the modulatory method of the invention involves contacting a cell expressing a gain-of-function mutant with a therapeutic agent (e.g., a RNAi agent or vector or transgene encoding same) that is specific for one or more target sequences within the gene, such that sequence specific interference with the gene is achieved. These methods can be performed in vitro (e.g., by culturing the cell with the agent) or, alternatively, in vivo (e.g., by administering the agent to a subject).

An RNA silencing agent modified for enhance uptake into neural cells can be administered at a unit dose less than about 1.4 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.00075, 0.00015 nmole of RNA silencing agent per kg of bodyweight. The unit dose, for example, can be administered by injection (e.g., intravenous or intramuscular, intrathecally, or directly into the brain), an inhaled dose, or a topical application. Particularly preferred dosages are less than 2, 1, or 0.1 mg/kg of body weight.

Delivery of an RNA silencing agent directly to an organ (e.g., directly to the brain, spinal column, placenta, liver and/or kidneys) can be at a dosage on the order of about 0.00001 mg to about 3 mg per organ, or preferably about 0.0001-0.001 mg per organ, about 0.03-3.0 mg per organ, about 0.1-3.0 mg per eye or about 0.3-3.0 mg per organ. The dosage can be an amount effective to treat or prevent a neurological disease or disorder (e.g., Huntington's disease) or a liver-, kidney- or pregnancy-related disease or disorder (e.g., PE, postpartum PE, eclampsia and/or HELLP). In one embodiment, the unit dose is administered less frequently than once a day, e.g., less than every 2, 4, 8 or 30 days. In another embodiment, the unit dose is not administered with a frequency (e.g., not a regular frequency). For example, the unit dose may be administered a single time. In one embodiment, the effective dose is administered with other traditional therapeutic modalities.

In one embodiment, a subject is administered an initial dose, and one or more maintenance doses of an RNA silencing agent. The maintenance dose or doses are generally lower than the initial dose, e.g., one-half less of the initial dose. A maintenance regimen can include treating the subject with a dose or doses ranging from 0.01 μg to 1.4 mg/kg of body weight per day, e.g., 10, 1, 0.1, 0.01, 0.001, or 0.00001 mg per kg of bodyweight per day. The maintenance doses are preferably administered no more than once every 5, 10, or 30 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

In one aspect, provided herein is a method of treating or managing preeclampsia, post-partum preeclampsia, eclampsia or HELLP syndrome comprising administering to a subject in need of such treatment or management a therapeutically effective amount of a compound, oligonucleotide, or nucleic acid as described herein, or a pharmaceutical composition comprising said compound, oligonucleotide, or nucleic acid.

In another aspect, provided herein is a method of treating or managing Huntington's disease comprising administering to a patient in need of such treatment or management a therapeutically effective amount of a compound, oligonucleotide, or nucleic acid as described herein, or a pharmaceutical composition comprising said compound, oligonucleotide, or nucleic acid.

### Definitions

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein in the context of oligonucleotide sequences, “A” represents a nucleoside comprising the base adenine (e.g., adenosine or a chemically-modified derivative thereof), “G” represents a nucleoside comprising the base guanine (e.g., guanosine or a chemically-modified derivative thereof), “U” represents a nucleoside comprising the base uracil (e.g., uridine or a chemically-modified derivative thereof), and “C” represents a nucleoside comprising the base adenine (e.g., cytidine or a chemically-modified derivative thereof),

As used herein, the terms “DHAPCL-hsiRNA,” “PC-DHA-hsiRNA,” “g2DHA-hsiRNA,” and “DHA-G2-hsiRNA” refer to an embodiment of compound (1) wherein X^{c} is DHA, L is L1 and O is a fully chemically modified as described herein.

As used herein, the term “capping group” refers to a chemical moiety that replaces a hydrogen atom in a functional group such as an alcohol (ROH), a carboxylic acid (RCO₂H), or an amine (RNH₂). Non-limiting examples of capping groups include: alkyl (e.g., methyl, tertiary-butyl); alkenyl (e.g., vinyl, allyl); carboxyl (e.g., acetyl, benzoyl); carbamoyl; phosphate; and phosphonate (e.g., vinylphosphonate). Other suitable capping groups are known to those of skill in the art.

By “trophoblast” is meant the mesectodermal cell layer covering the blastocyst that erodes the uterine mucosa and through which the embryo receives nourishment from the mother. Trophoblast cells contribute to the formation of the placenta.

The term “nucleotide analog” or “altered nucleotide” or “modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide which may be derivatized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Linkers useful in conjugated compounds of the invention include glycol chains (e.g., polyethylene glycol), alkyl chains, peptides, RNA, DNA, and combinations thereof. As used herein, the abbreviation “TEG” refers to triethylene glycol.

Nucleotide analogs may also comprise modifications to the sugar portion of the nucleotides. For example the 2′ OH-group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vitro.

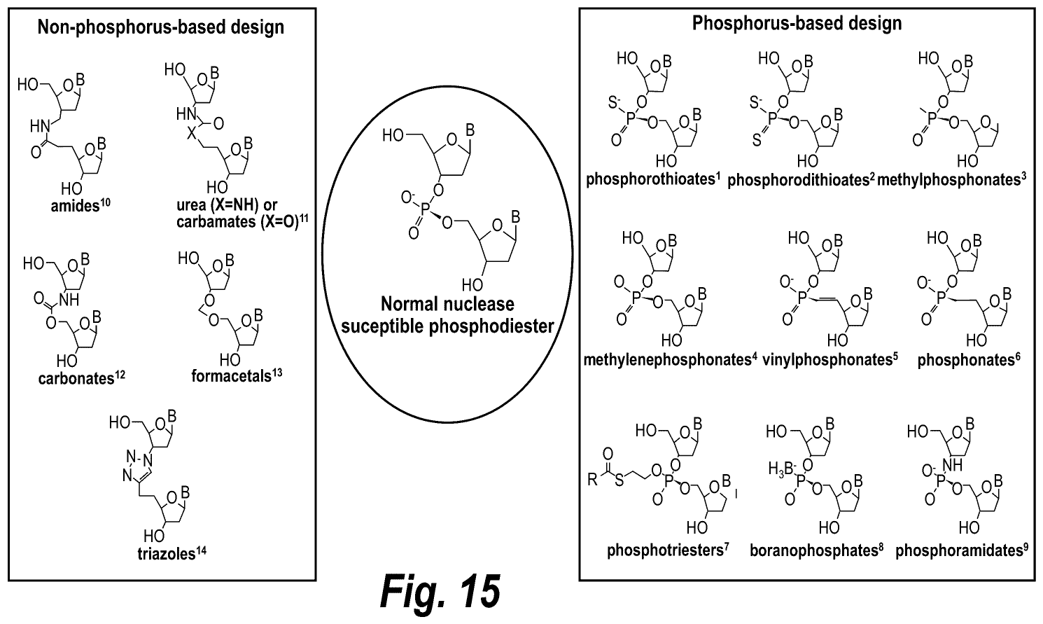

In some embodiments, the compounds, oligonucleotides and nucleic acids described herein may be modified to comprise the internucleotide linkages provided in FIG. 15. In particular embodiments, the compounds, oligonucleotides and nucleic acids described herein comprise internucleotide linkages selected from phosphodiester and phosphorothioate.

It is understood that certain internucleotide linkages provided herein, including, e.g., phosphodiester and phosphorothioate, comprise a formal charge of −1 at physiological pH, and that said formal charge will be balanced by a cationic moiety, e.g., an alkali metal such as sodium or potassium, an alkali earth metal such as calcium or magnesium, or an ammonium or guanidinium ion.

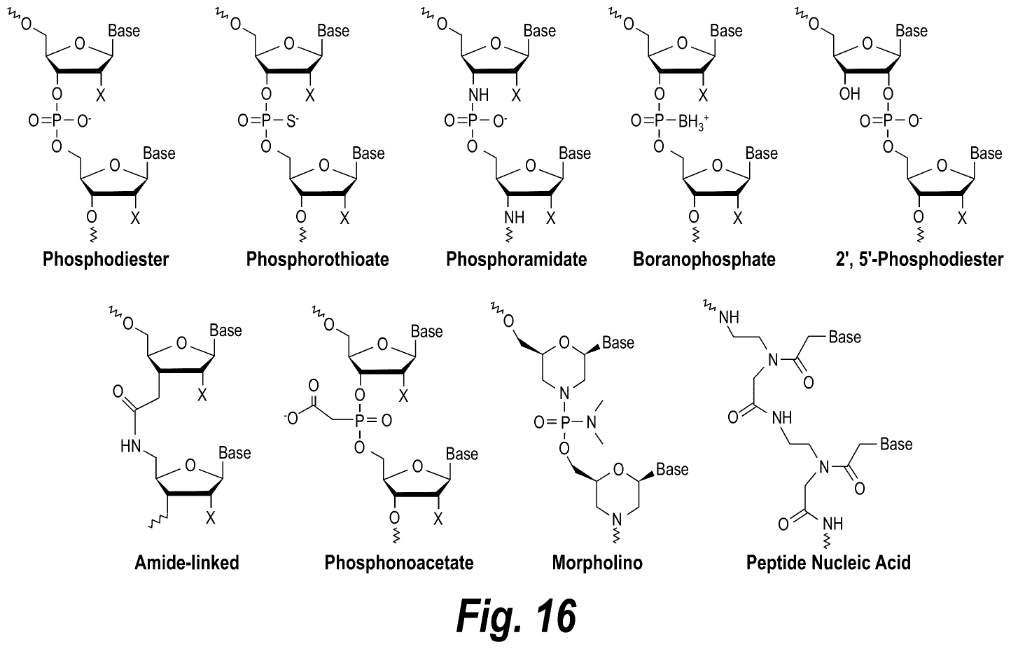

Oligonucleotide backbones may comprise phosphates, phosphorothioates (a racemic mixture or stereospecific), diphosphorothioates, phosphoramidates, peptide nucleic acid, boranophosphate, 2′-5′ phosphodiester, amides, phosphonoacetate, morpholino moieties, or a combination thereof. In some embodiments, the compounds, oligonucleotides and nucleic acids described herein may be modified to comprise the internucleotide backbone linkages provided in FIG. 16.

In certain embodiments, provided herein are compounds comprising a phosphate moiety (e.g., X1, X4, X5 and X6), a phosphonate moiety (e.g., X3, X7 and X8). These moieties will be partially or completely ionized as a function of the moiety's pKa and the pH of the environment. It is understood that negatively charged ions will be balanced by a cationic moiety, e.g., an alkali metal such as sodium or potassium, an alkali earth metal such as calcium or magnesium, or an ammonium or guanidinium ion.

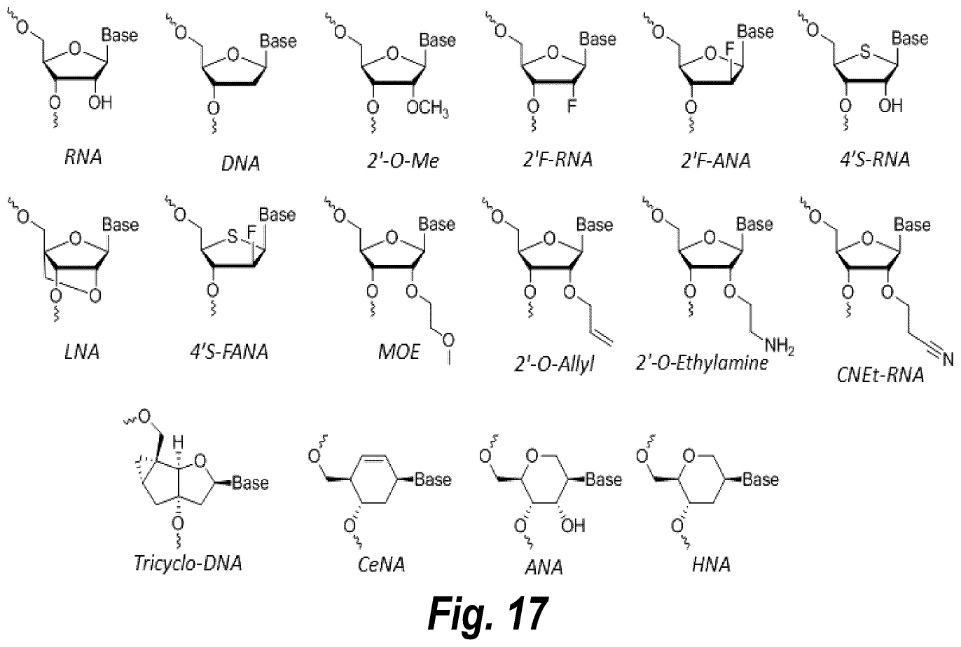

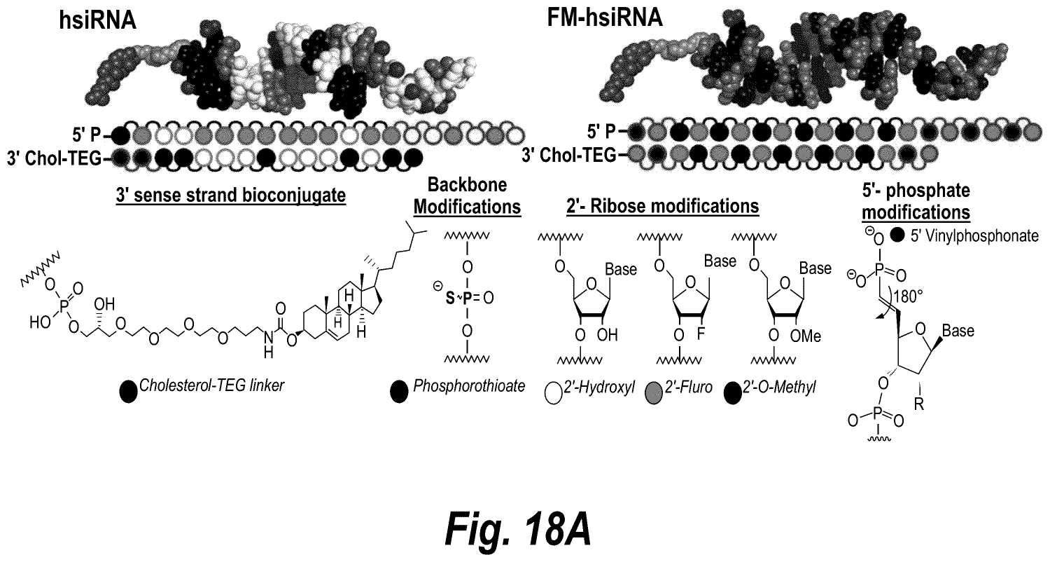

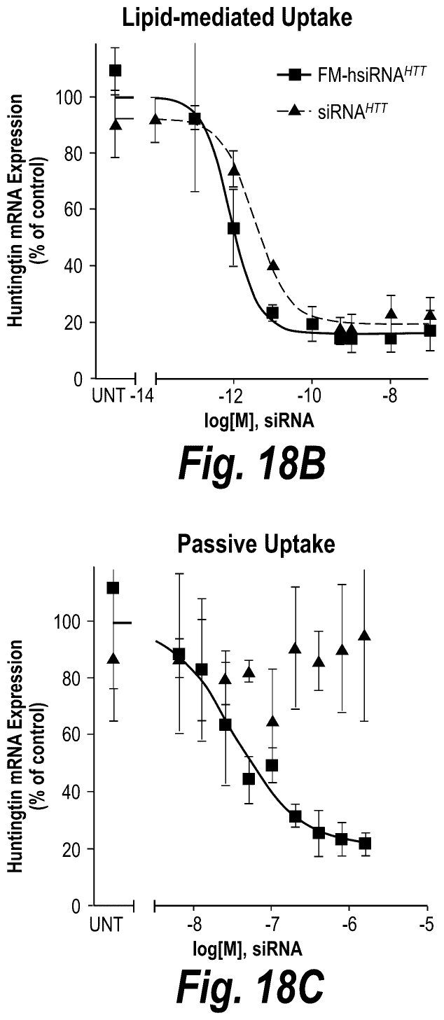

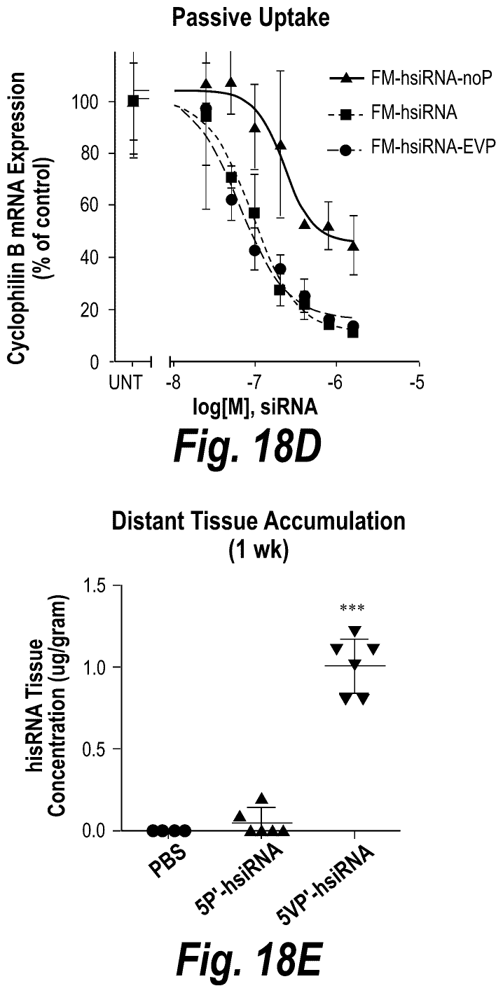

In some embodiments, the compounds, oligonucleotides and nucleic acids described herein may be modified to comprise the sugar modifications provided in FIG. 17.

### Methods of Delivering Nucleic Acid

In another aspect, provided herein is a method for selectively delivering a compound or nucleic acid as described herein to a particular organ in a patient, comprising administering said nucleic acid to the patient, wherein the nucleic acid comprises a bioactive molecule having an affinity for a receptor. In one embodiment, the organ is the liver. In another embodiment, the organ is the kidneys. In another embodiment, the organ is the spleen. In another embodiment, the organ is the heart. In another embodiment, the organ is the brain.

The nature of the conjugated hydrophobic moiety (e.g., DHA and EPA) dramatically alters tissue distribution profiles. In certain embodiments, cholesterol and saturated fatty acid (e.g., DCA)-conjugated hsiRNA distributes preferentially to the liver and spleen. In other embodiments, polyunsaturated fatty acid (e.g., DHA and EPA)-conjugated hsiRNA distributes preferentially to the kidneys and heart in addition to the liver and spleen. In a particular embodiment, DHA-conjugated hsiRNA distributes preferentially to the kidneys. In another particular embodiment, the delivery of DHA-conjugated hsiRNA to the kidneys is specific to proximal tubule cells, preferentially involved in a range of kidney diseases including diabetic nephropathy, renal cancer, and lupus. DHA-conjugated hsiRNA shows robust gene modulation in the liver and kidney after a single IV injection of 15 mg/kg.

As shown in FIG. 21, highly hydrophobic siRNA conjugates (e.g. cholesterol, docosanoic acid) distribute primarily to the liver after systemic (intravenous or subcutaneous) delivery, with residual accumulation in the spleen. Less hydrophobic siRNA conjugates (e.g. polyunsaturated fatty acids such as docosahexaenoic acid and eicosapentaenoic acid) distribute to the kidney, liver, and heart after systemic delivery. This distribution pattern correlates with the observed efficacy of this panel of conjugates in the liver, where Chol- and DCA-siRNA are highly accumulated and show higher silencing (˜70%), while DHA- and EPA-siRNA conjugate accumulation is less pronounced and therefore shows lower levels of silencing (40% and 25%, respectively). An siRNA containing the tetraethylene glycol linker only (Linker only) shows residual levels of liver silencing as well.

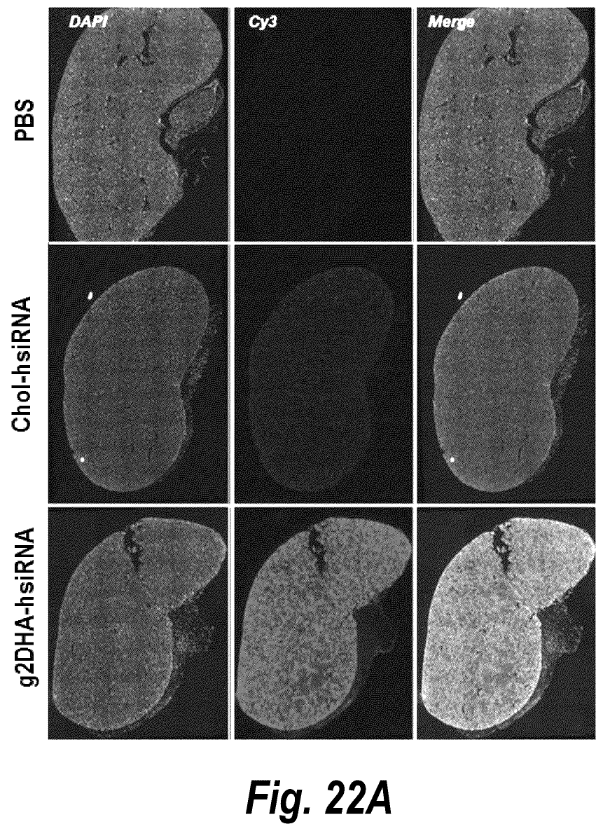

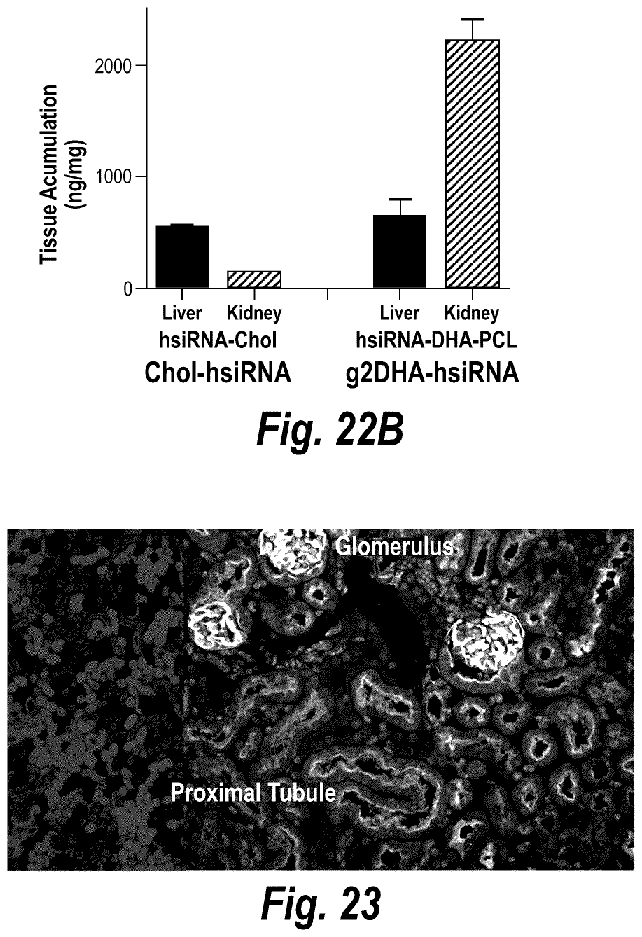

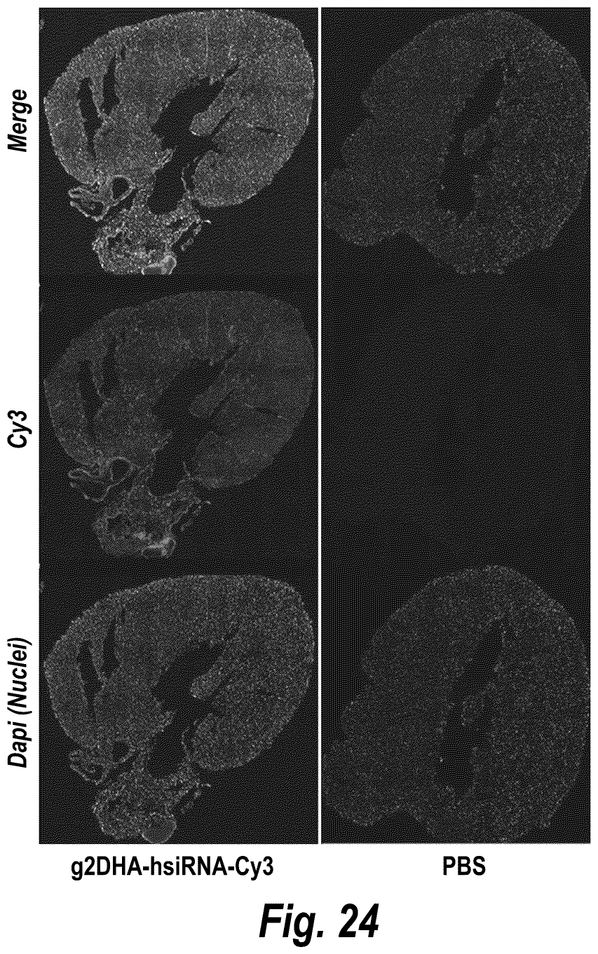

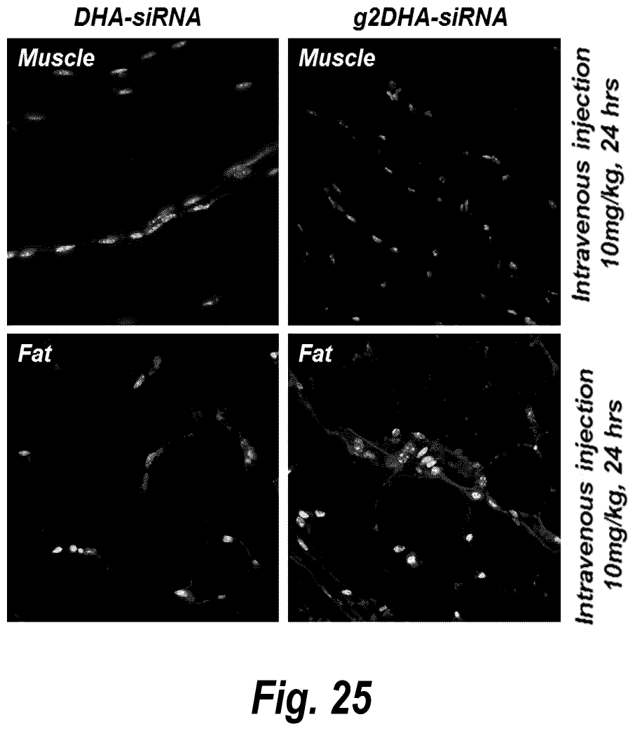

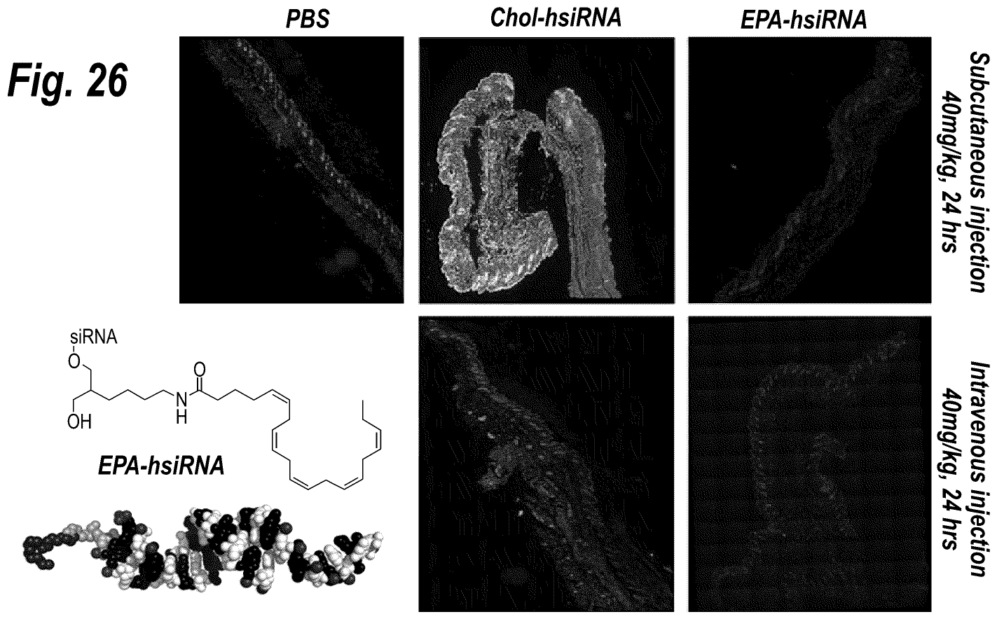

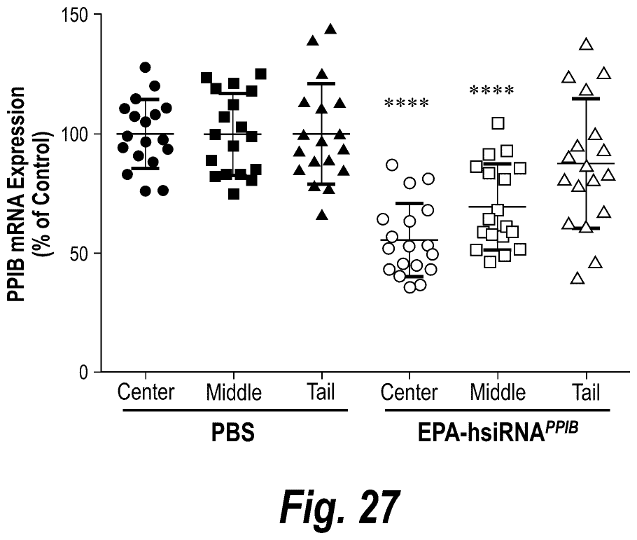

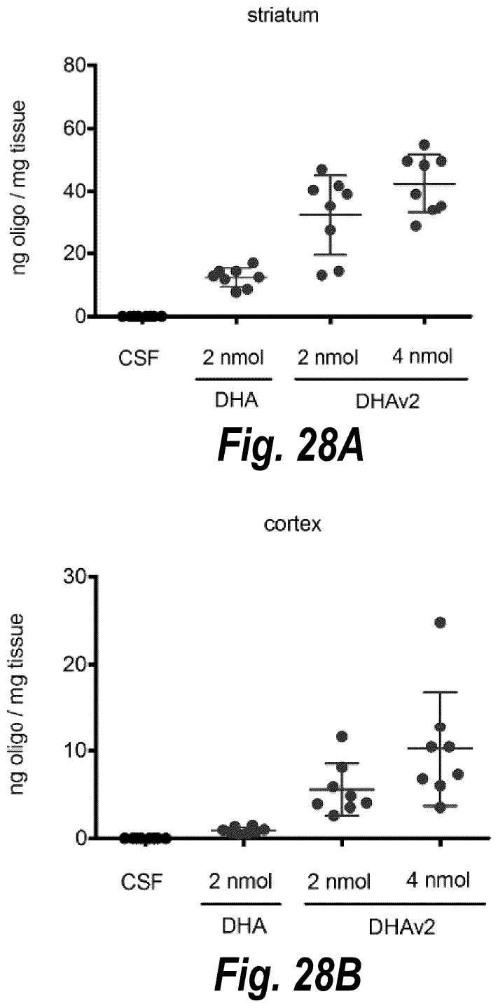

As shown in FIG. 22, g2DHA-siRNA shows preferential localization in the kidney following a single, intravenous injection, which directly contrasts the typical liver distribution observed for highly hydrophobic lipid-siRNA conjugates (e.g. cholesterol, DCA). The differences in the degree of accumulation was measured using a quantitative peptide nucleic acid hybridization assay. We observe a statistically significant increase in kidney accumulation and decrease in liver accumulation with g2DHA-siRNA compared to Chol-siRNA.

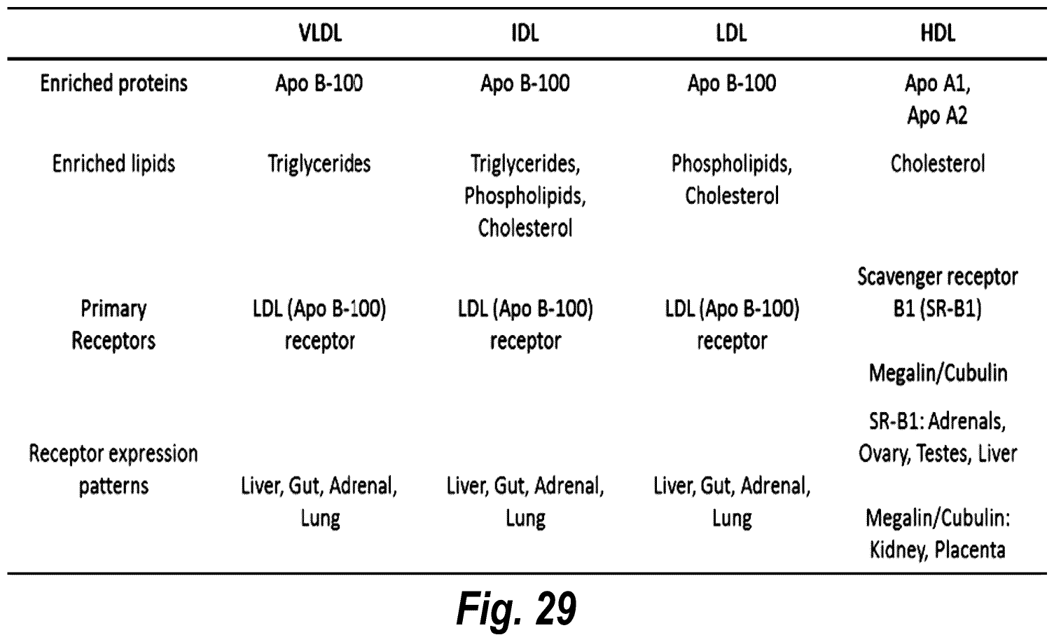

Serum lipoprotein complexes are responsible for trafficking endogenous fatty acids and lipids throughout the bloodstream. Lipid-conjugated siRNAs may avail themselves of this mechanism to achieve distribution to different tissues following intravenous administration. FIG. 29 describes the different lipid-binding and systemic distribution characteristics of each individual serum lipoprotein. Very low density lipoprotein (VLDL); Intermediate density lipoprotein (IDL); Low density lipoprotein (LDL); High density lipoprotein (HDL).

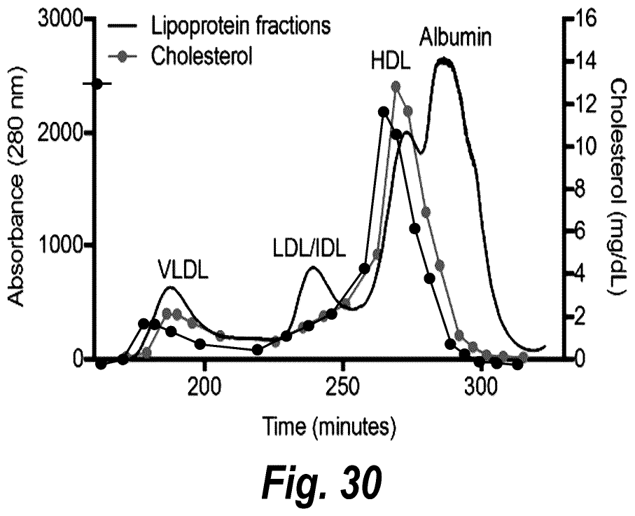

The different tissue distribution patterns observed in vivo for each distinct siRNA conjugate are determined by their lipoprotein binding profiles. These profiles can be determined empirically using size exclusion chromatography and monitoring the absorbance at 280 nm (protein). As shown in FIG. 30, protein peak fractions were collected and a cholesterol quantification assay was used to determine the identity of each peak in the trace. In wild-type FVB/NJ mice, cholesterol is primarily associated with HDL. From this, the albumin, HDL, LDL/IDL, and VLDL peaks were assigned.

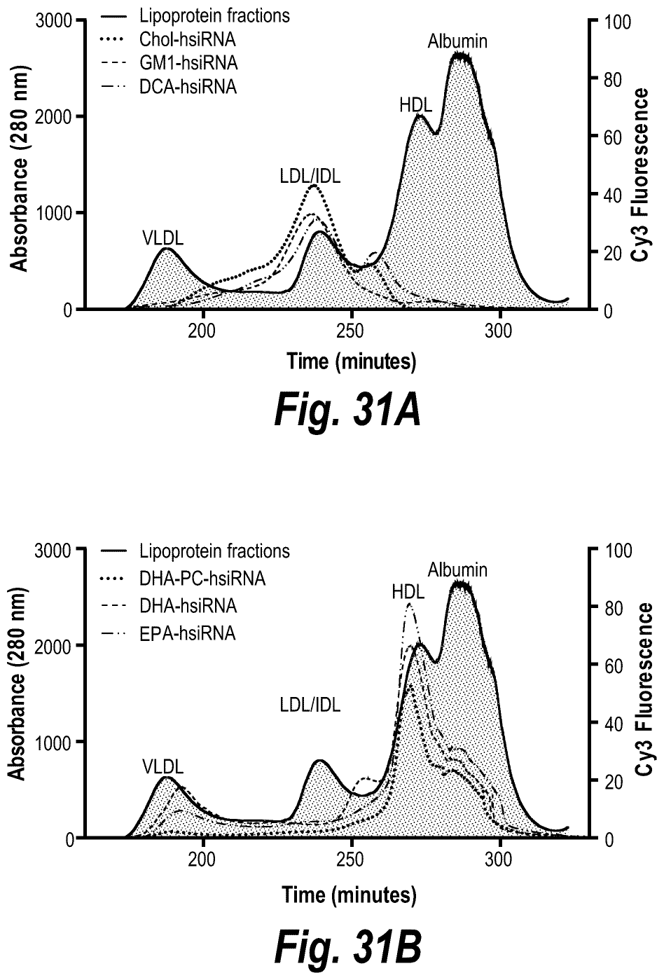

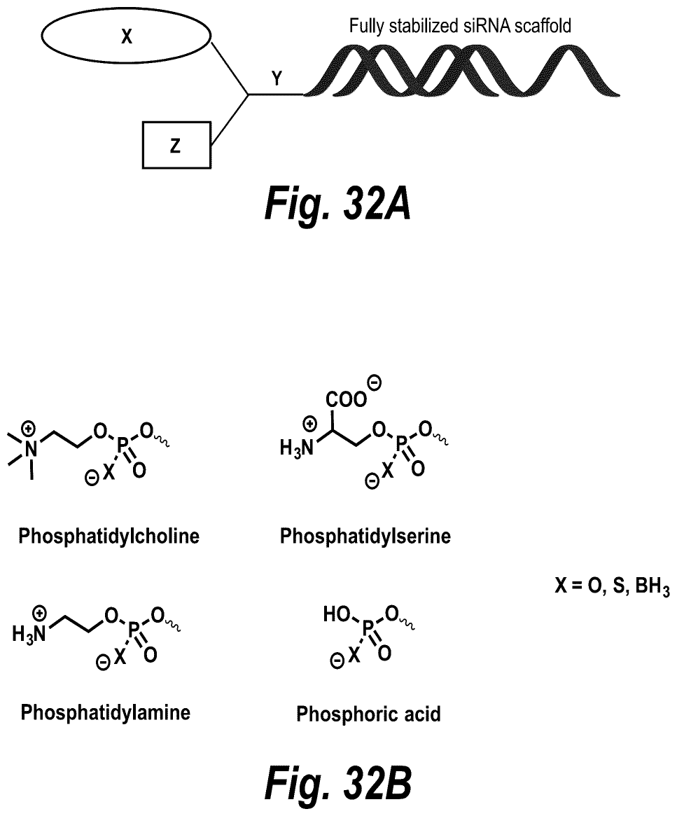

The serum lipoprotein profile of siRNA in mouse blood was analyzed. As shown in FIG. 31, Cy3-labeled siRNA conjugates were incubated ex vivo with serum isolated from wild type mice and analyzed as described previously by size exclusion chromatography. This lipoprotein binding correlates with observed PK/PD and distribution to the liver, kidney, and spleen (primarily VLDL, LDL, and IDL binding) or kidney, liver, and heart (HDL binding). Below, we demonstrate that cholesterol, DCA, and GM1 conjugates preferentially associate with IDL and LDL, while EPA, DHA, and DHAg2 conjugates preferentially associate with HDL. For polyunsaturated fatty acid-siRNA conjugates, the minimum number of double bonds necessary to achieve HDL binding and distribution to the kidney is >=3 (e.g. DHA, EPA, anandamide, alpha-linolenic acid, gamma-linolenic acid, arachidonic acid, etc.).

### DHA Conjugation

Direct conjugation of DHA to a fully chemically stabilized siRNA scaffold shows significant tissue retention with wide distribution and robust efficacy in mouse brain. Notably, DHA-hsiRNA conjugates do not elicit measurable microglial activation and have no adverse effect on neuronal viability at concentrations over 20-fold higher than the efficacious dose.

DHA-hsiRNA alleviates one of the major obstacles to neurological applications of siRNA, which is achieving widespread brain distribution. Following a direct intrastriatal injection, DHA-hsiRNA distributed broadly throughout the striatum and cortex of the injected hemisphere, with no dramatic compound accumulation around the site of injection (a typical feature of Chol-hsiRNA). DHA-hsiRNA co-localizes with both neuronal (NeuN) and astrocyte (GFAP) markers. DHA-hsiRNA clearly localized to the perinuclear space in both striatal and cortical neurons (the cytoplasmic site of active RNAi).

Comparing increasing concentrations of DHA-hsiRNA and Chol-hsiRNA, it was found that Chol-hsiRNA induced significant loss of brain matter and occasionally animal morbidity at doses above 25 μg. In contrast, animals injected with 200 μg of DHA-hsiRNA appeared healthy, with normal brain morphology. 200 μg is the maximal amount that can be delivered intrastriatally, given the solubility limit of DHA-hsiRNA.

Design of siRNA Molecules

In some embodiments, an siRNA molecule of the invention is a duplex consisting of a sense strand and complementary antisense strand, the antisense strand having sufficient complementary to an htt mRNA to mediate RNAi. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is sufficiently complementary to a target region. Preferably, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is substantially complementary to a target sequence, and the other strand is identical or substantially identical to the first strand.

Generally, siRNAs can be designed by using any method known in the art, for instance, by using the following protocol:

1. The siRNA may be specific for a target sequence. Preferably, the first strand is substantially complementary to the target sequence, and the other strand is substantially complementary to the first strand. In an embodiment, the target sequence is outside a coding region of the target gene. Exemplary target sequences are selected from the 5′ untranslated region (5′-UTR) or an intronic region of a target gene. Cleavage of mRNA at these sites should eliminate translation of corresponding mutant protein. Target sequences from other regions of the htt gene are also suitable for targeting. A sense strand is designed based on the target sequence. Further, siRNAs with lower G/C content (35-55%) may be more active than those with G/C content higher than 55%. Thus in one embodiment, the invention includes nucleic acid molecules having 35-55% G/C content.

2. The sense strand of the siRNA is designed based on the sequence of the selected target site. Preferably the RNA silencing agents of the invention do not elicit a PKR response (i.e., are of a sufficiently short length). However, longer RNA silencing agents may be useful, for example, in cell types incapable of generating a PRK response or in situations where the PKR response has been down-regulated or dampened by alternative means.

The siRNA molecules of the invention have sufficient complementarity with the target sequence such that the siRNA can mediate RNAi. In general, siRNA containing nucleotide sequences sufficiently identical to a target sequence portion of the target gene to effect RISC-mediated cleavage of the target gene are preferred. Accordingly, in a preferred embodiment, the sense strand of the siRNA is designed have to have a sequence sufficiently identical to a portion of the target. For example, the sense strand may have 100% identity to the target site. However, 100% identity is not required. Greater than 80% identity, e.g., 80%, 81%, 82%, 83%, 84%, 85%, 86%, 87%, 88%, 89%, 90%, 91%, 92%, 93%, 94%, 95%, 96%, 97%, 98%, 99% or even 100% identity, between the sense strand and the target RNA sequence is preferred. The invention has the advantage of being able to tolerate certain sequence variations to enhance efficiency and specificity of RNAi. In one embodiment, the sense strand has 4, 3, 2, 1, or 0 mismatched nucleotide(s) with a target region, such as a target region that differs by at least one base pair between a wild-type and mutant allele, e.g., a target region comprising the gain-of-function mutation, and the other strand is identical or substantially identical to the first strand. Moreover, siRNA sequences with small insertions or deletions of 1 or 2 nucleotides may also be effective for mediating RNAi. Alternatively, siRNA sequences with nucleotide analog substitutions or insertions can be effective for inhibition.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., percent (%) homology=number of identical positions/total number of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

3. The antisense or guide strand of the siRNA is routinely the same length as the sense strand and includes complementary nucleotides. In one embodiment, the guide and sense strands are fully complementary, i.e., the strands are blunt-ended when aligned or annealed. In another embodiment, the strands of the siRNA can be paired in such a way as to have a 3′ overhang of 1 to 4, e.g., 2, nucleotides. Overhangs can comprise (or consist of) nucleotides corresponding to the target gene sequence (or complement thereof). Alternatively, overhangs can comprise (or consist of) deoxyribonucleotides, for example dTs, or nucleotide analogs, or other suitable non-nucleotide material. Thus in another embodiment, the nucleic acid molecules may have a 3′ overhang of 2 nucleotides, such as TT. The overhanging nucleotides may be either RNA or DNA. As noted above, it is desirable to choose a target region wherein the mutant:wild type mismatch is a purine:purine mismatch.

4. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website.

5. Select one or more sequences that meet the criteria for evaluation.

Further general information about the design and use of siRNA may be found in “The siRNA User Guide,” available at The Max-Plank-Institut fur Biophysikalishe Chemie website.

Alternatively, the siRNA may be defined functionally as a nucleotide sequence (or oligonucleotide sequence) that is capable of hybridizing with the target sequence (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G-FC bases). For hybrids between 18 and 49 base pairs in length, Tm(° C.)=81.5+16.6(log 10[Na+])+0.41(% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Negative control siRNAs should have the same nucleotide composition as the selected siRNA, but without significant sequence complementarity to the appropriate genome. Such negative controls may be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

6. To validate the effectiveness by which siRNAs destroy target mRNAs (e.g., wild-type or mutant huntingtin mRNA), the siRNA may be incubated with target cDNA (e.g., huntingtin cDNA) in a Drosophila-based in vitro mRNA expression system. Radiolabeled with ³²P, newly synthesized target mRNAs (e.g., huntingtin mRNA) are detected autoradiographically on an agarose gel. The presence of cleaved target mRNA indicates mRNA nuclease activity. Suitable controls include omission of siRNA and use of non-target cDNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

siRNAs may be designed to target any of the target sequences described supra. Said siRNAs comprise an antisense strand which is sufficiently complementary with the target sequence to mediate silencing of the target sequence. In certain embodiments, the RNA silencing agent is a siRNA.

In certain embodiments, the siRNA comprises a sense strand comprising a sequence set forth in FIG. 14, and an antisense strand comprising a sequence set forth in FIG. 14.

Sites of siRNA-mRNA complementation are selected which result in optimal mRNA specificity and maximal mRNA cleavage.

siRNA-Like Molecules

siRNA-like molecules of the invention have a sequence (i.e., have a strand having a sequence) that is “sufficiently complementary” to a target sequence of an mRNA (e.g. htt mRNA) to direct gene silencing either by RNAi or translational repression. siRNA-like molecules are designed in the same way as siRNA molecules, but the degree of sequence identity between the sense strand and target RNA approximates that observed between an miRNA and its target. In general, as the degree of sequence identity between a miRNA sequence and the corresponding target gene sequence is decreased, the tendency to mediate post-transcriptional gene silencing by translational repression rather than RNAi is increased. Therefore, in an alternative embodiment, where post-transcriptional gene silencing by translational repression of the target gene is desired, the miRNA sequence has partial complementarity with the target gene sequence. In certain embodiments, the miRNA sequence has partial complementarity with one or more short sequences (complementarity sites) dispersed within the target mRNA (e.g. within the 3′-UTR of the target mRNA) (Hutvagner and Zamore, Science, 2002; Zeng et al., Mol. Cell, 2002; Zeng et al., RNA, 2003; Doench et al., Genes & Dev., 2003). Since the mechanism of translational repression is cooperative, multiple complementarity sites (e.g., 2, 3, 4, 5, or 6) may be targeted in certain embodiments.

The capacity of a siRNA-like duplex to mediate RNAi or translational repression may be predicted by the distribution of non-identical nucleotides between the target gene sequence and the nucleotide sequence of the silencing agent at the site of complementarity. In one embodiment, where gene silencing by translational repression is desired, at least one non-identical nucleotide is present in the central portion of the complementarity site so that duplex formed by the miRNA guide strand and the target mRNA contains a central “bulge” (Doench J G et al., Genes & Dev., 2003). In another embodiment 2, 3, 4, 5, or 6 contiguous or non-contiguous non-identical nucleotides are introduced. The non-identical nucleotide may be selected such that it forms a wobble base pair (e.g., G:U) or a mismatched base pair (G:A, C:A, C:U, G:G, A:A, C:C, U:U). In a further preferred embodiment, the “bulge” is centered at nucleotide positions 12 and 13 from the 5′ end of the miRNA molecule.

### Modified RNA Silencing Agents

In certain aspects of the invention, an RNA silencing agent (or any portion thereof) of the invention as described supra may be modified such that the activity of the agent is further improved. For example, the RNA silencing agents described in above may be modified with any of the modifications described infra. The modifications can, in part, serve to further enhance target discrimination, to enhance stability of the agent (e.g., to prevent degradation), to promote cellular uptake, to enhance the target efficiency, to improve efficacy in binding (e.g., to the targets), to improve patient tolerance to the agent, and/or to reduce toxicity.

### 1) Modifications to Enhance Target Discrimination

In certain embodiments, the RNA silencing agents of the invention may be substituted with a destabilizing nucleotide to enhance single nucleotide target discrimination (see U.S. application Ser. No. 11/698,689, filed Jan. 25, 2007 and U.S. Provisional Application No. 60/762,225 filed Jan. 25, 2006, both of which are incorporated herein by reference). Such a modification may be sufficient to abolish the specificity of the RNA silencing agent for a non-target mRNA (e.g. wild-type mRNA), without appreciably affecting the specificity of the RNA silencing agent for a target mRNA (e.g. gain-of-function mutant mRNA).

In preferred embodiments, the RNA silencing agents of the invention are modified by the introduction of at least one universal nucleotide in the antisense strand thereof. Universal nucleotides comprise base portions that are capable of base pairing indiscriminately with any of the four conventional nucleotide bases (e.g. A, G, C, U). A universal nucleotide is preferred because it has relatively minor effect on the stability of the RNA duplex or the duplex formed by the guide strand of the RNA silencing agent and the target mRNA. Exemplary universal nucleotide include those having an inosine base portion or an inosine analog base portion selected from the group consisting of deoxyinosine (e.g. 2′-deoxyinosine), 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. In particularly preferred embodiments, the universal nucleotide is an inosine residue or a naturally occurring analog thereof.

In certain embodiments, the RNA silencing agents of the invention are modified by the introduction of at least one destabilizing nucleotide within 5 nucleotides from a specificity-determining nucleotide (i.e., the nucleotide which recognizes the disease-related polymorphism). For example, the destabilizing nucleotide may be introduced at a position that is within 5, 4, 3, 2, or 1 nucleotide(s) from a specificity-determining nucleotide. In exemplary embodiments, the destabilizing nucleotide is introduced at a position which is 3 nucleotides from the specificity-determining nucleotide (i.e., such that there are 2 stabilizing nucleotides between the destablilizing nucleotide and the specificity-determining nucleotide). In RNA silencing agents having two strands or strand portions (e.g. siRNAs and shRNAs), the destabilizing nucleotide may be introduced in the strand or strand portion that does not contain the specificity-determining nucleotide. In preferred embodiments, the destabilizing nucleotide is introduced in the same strand or strand portion that contains the specificity-determining nucleotide.

### 2) Modifications to Enhance Efficacy and Specificity

In certain embodiments, the RNA silencing agents of the invention may be altered to facilitate enhanced efficacy and specificity in mediating RNAi according to asymmetry design rules (see U.S. Pat. Nos. 8,309,704, 7,750,144, 8,304,530, 8,329,892 and 8,309,705). Such alterations facilitate entry of the antisense strand of the siRNA (e.g., a siRNA designed using the methods of the invention or an siRNA produced from a shRNA) into RISC in favor of the sense strand, such that the antisense strand preferentially guides cleavage or translational repression of a target mRNA, and thus increasing or improving the efficiency of target cleavage and silencing. Preferably the asymmetry of an RNA silencing agent is enhanced by lessening the base pair strength between the antisense strand 5′ end (AS 5′) and the sense strand 3′ end (S 3′) of the RNA silencing agent relative to the bond strength or base pair strength between the antisense strand 3′ end (AS 3′) and the sense strand 5′ end (S ′5) of said RNA silencing agent.

In one embodiment, the asymmetry of an RNA silencing agent of the invention may be enhanced such that there are fewer G:C base pairs between the 5′ end of the first or antisense strand and the 3′ end of the sense strand portion than between the 3′ end of the first or antisense strand and the 5′ end of the sense strand portion. In another embodiment, the asymmetry of an RNA silencing agent of the invention may be enhanced such that there is at least one mismatched base pair between the 5′ end of the first or antisense strand and the 3′ end of the sense strand portion. Preferably, the mismatched base pair is selected from the group consisting of G:A, C:A, C:U, G:G, A:A, C:C and U:U. In another embodiment, the asymmetry of an RNA silencing agent of the invention may be enhanced such that there is at least one wobble base pair, e.g., G:U, between the 5′ end of the first or antisense strand and the 3′ end of the sense strand portion. In another embodiment, the asymmetry of an RNA silencing agent of the invention may be enhanced such that there is at least one base pair comprising a rare nucleotide, e.g., inosine (I). Preferably, the base pair is selected from the group consisting of an I:A, I:U and I:C. In yet another embodiment, the asymmetry of an RNA silencing agent of the invention may be enhanced such that there is at least one base pair comprising a modified nucleotide. In preferred embodiments, the modified nucleotide is selected from the group consisting of 2-amino-G, 2-amino-A, 2,6-diamino-G, and 2,6-diamino-A.

3) RNA Silencing Agents with Enhanced Stability

The RNA silencing agents of the present invention can be modified to improve stability in serum or in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference.

In a preferred aspect, the invention features RNA silencing agents that include first and second strands wherein the second strand and/or first strand is modified by the substitution of internal nucleotides with modified nucleotides, such that in vivo stability is enhanced as compared to a corresponding unmodified RNA silencing agent. As defined herein, an “internal” nucleotide is one occurring at any position other than the 5′ end or 3′ end of nucleic acid molecule, polynucleotide or oligonucleotide. An internal nucleotide can be within a single-stranded molecule or within a strand of a duplex or double-stranded molecule. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the internal nucleotides.

In a preferred embodiment of the present invention, the RNA silencing agents may contain at least one modified nucleotide analogue. The nucleotide analogues may be located at positions where the target-specific silencing activity, e.g., the RNAi mediating activity or translational repression activity is not substantially effected, e.g., in a region at the 5′-end and/or the 3′-end of the siRNA molecule. Particularly, the ends may be stabilized by incorporating modified nucleotide analogues.

Exemplary nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides, the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

In particular embodiments, the modifications are 2′-fluoro, 2′-amino and/or 2′-thio modifications. Particularly preferred modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a particular embodiment, the 2′-fluoro ribonucleotides are every uridine and cytidine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. 2′-deoxy-nucleotides and 2′-Ome nucleotides can also be used within modified RNA-silencing agents moities of the instant invention. Additional modified residues include, deoxy-abasic, inosine, N3-methyl-uridine, N6,N6-dimethyl-adenosine, pseudouridine, purine ribonucleoside and ribavirin. In a particularly preferred embodiment, the 2′ moiety is a methyl group such that the linking moiety is a 2′-O-methyl oligonucleotide.

In an exemplary embodiment, the RNA silencing agent of the invention comprises Locked Nucleic Acids (LNAs). LNAs comprise sugar-modified nucleotides that resist nuclease activities (are highly stable) and possess single nucleotide discrimination for mRNA (Elmen et al., Nucleic Acids Res., (2005), 33(1): 439-447; Braasch et al. (2003) Biochemistry 42:7967-7975, Petersen et al. (2003) Trends Biotechnol 21:74-81). These molecules have 2′-O,4′-C-ethylene-bridged nucleic acids, with possible modifications such as 2′-deoxy-2″-fluorouridine. Moreover, LNAs increase the specificity of oligonucleotides by constraining the sugar moiety into the 3′-endo conformation, thereby pre-organizing the nucleotide for base pairing and increasing the melting temperature of the oligonucleotide by as much as 10° C. per base.

In another exemplary embodiment, the RNA silencing agent of the invention comprises Peptide Nucleic Acids (PNAs). PNAs comprise modified nucleotides in which the sugar-phosphate portion of the nucleotide is replaced with a neutral 2-amino ethylglycine moiety capable of forming a polyamide backbone which is highly resistant to nuclease digestion and imparts improved binding specificity to the molecule (Nielsen, et al., Science, (2001), 254: 1497-1500).

Also preferred are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In other embodiments, cross-linking can be employed to alter the pharmacokinetics of the RNA silencing agent, for example, to increase half-life in the body. Thus, the invention includes RNA silencing agents having two complementary strands of nucleic acid, wherein the two strands are crosslinked. The invention also includes RNA silencing agents which are conjugated or unconjugated (e.g., at its 3′ terminus) to another moiety (e.g. a non-nucleic acid moiety such as a peptide), an organic compound (e.g., a dye), or the like). Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

Other exemplary modifications include: (a) 2′ modification, e.g., provision of a 2′ OMe moiety on a U in a sense or antisense strand, but especially on a sense strand, or provision of a 2′ OMe moiety in a 3′ overhang, e.g., at the 3′ terminus (3′ terminus means at the 3′ atom of the molecule or at the most 3′ moiety, e.g., the most 3′ P or 2′ position, as indicated by the context); (b) modification of the backbone, e.g., with the replacement of an 0 with an S, in the phosphate backbone, e.g., the provision of a phosphorothioate modification, on the U or the A or both, especially on an antisense strand; e.g., with the replacement of a P with an S; (c) replacement of the U with a C5 amino linker; (d) replacement of an A with a G (sequence changes are preferred to be located on the sense strand and not the antisense strand); and (d) modification at the 2′, 6′, 7′, or 8′ position. Exemplary embodiments are those in which one or more of these modifications are present on the sense but not the antisense strand, or embodiments where the antisense strand has fewer of such modifications. Yet other exemplary modifications include the use of a methylated P in a 3′ overhang, e.g., at the 3′ terminus; combination of a 2′ modification, e.g., provision of a 2′ 0 Me moiety and modification of the backbone, e.g., with the replacement of a P with an S, e.g., the provision of a phosphorothioate modification, or the use of a methylated P, in a 3′ overhang, e.g., at the 3′ terminus; modification with a 3′ alkyl; modification with an abasic pyrrolidone in a 3′ overhang, e.g., at the 3′ terminus; modification with naproxen, ibuprofen, or other moieties which inhibit degradation at the 3′ terminus.

### 4) Modifications to Enhance Cellular Uptake

In other embodiments, RNA silencing agents may be modified with chemical moieties, for example, to enhance cellular uptake by target cells (e.g., neuronal cells). Thus, the invention includes RNA silencing agents which are conjugated or unconjugated (e.g., at its 3′ terminus) to another moiety (e.g. a non-nucleic acid moiety such as a peptide), an organic compound (e.g., a dye), or the like. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

In a particular embodiment, an RNA silencing agent of invention is conjugated to a lipophilic moiety. In one embodiment, the lipophilic moiety is a ligand that includes a cationic group. In another embodiment, the lipophilic moiety is attached to one or both strands of an siRNA. In an exemplary embodiment, the lipophilic moiety is attached to one end of the sense strand of the siRNA. In another exemplary embodiment, the lipophilic moiety is attached to the 3′ end of the sense strand. In certain embodiments, the lipophilic moiety is selected from the group consisting of cholesterol, vitamin E, vitamin K, vitamin A, folic acid, or a cationic dye (e.g., Cy3). In an exemplary embodiment, the lipophilic moiety is a cholesterol. Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

### 5) Tethered Ligands

Other entities can be tethered to an RNA silencing agent of the invention. For example, a ligand tethered to an RNA silencing agent to improve stability, hybridization thermodynamics with a target nucleic acid, targeting to a particular tissue or cell-type, or cell permeability, e.g., by an endocytosis-dependent or -independent mechanism. Ligands and associated modifications can also increase sequence specificity and consequently decrease off-site targeting. A tethered ligand can include one or more modified bases or sugars that can function as intercalators. These are preferably located in an internal region, such as in a bulge of RNA silencing agent/target duplex. The intercalator can be an aromatic, e.g., a polycyclic aromatic or heterocyclic aromatic compound. A polycyclic intercalator can have stacking capabilities, and can include systems with 2, 3, or 4 fused rings. The universal bases described herein can be included on a ligand. In one embodiment, the ligand can include a cleaving group that contributes to target gene inhibition by cleavage of the target nucleic acid. The cleaving group can be, for example, a bleomycin (e.g., bleomycin-A5, bleomycin-A2, or bleomycin-B2), pyrene, phenanthroline (e.g., O-phenanthroline), a polyamine, a tripeptide (e.g., lys-tyr-lys tripeptide), or metal ion chelating group. The metal ion chelating group can include, e.g., an Lu(III) or EU(III) macrocyclic complex, a Zn(II) 2,9-dimethylphenanthroline derivative, a Cu(II) terpyridine, or acridine, which can promote the selective cleavage of target RNA at the site of the bulge by free metal ions, such as Lu(III). In some embodiments, a peptide ligand can be tethered to a RNA silencing agent to promote cleavage of the target RNA, e.g., at the bulge region. For example, 1,8-dimethyl-1,3,6,8,10,13-hexaazacyclotetradecane (cyclam) can be conjugated to a peptide (e.g., by an amino acid derivative) to promote target RNA cleavage. A tethered ligand can be an aminoglycoside ligand, which can cause an RNA silencing agent to have improved hybridization properties or improved sequence specificity. Exemplary aminoglycosides include glycosylated polylysine, galactosylated polylysine, neomycin B, tobramycin, kanamycin A, and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. An acridine analog, neo-5-acridine has an increased affinity for the HIV Rev-response element (RRE). In some embodiments the guanidine analog (the guanidinoglycoside) of an aminoglycoside ligand is tethered to an RNA silencing agent. In a guanidinoglycoside, the amine group on the amino acid is exchanged for a guanidine group. Attachment of a guanidine analog can enhance cell permeability of an RNA silencing agent. A tethered ligand can be a poly-arginine peptide, peptoid or peptidomimetic, which can enhance the cellular uptake of an oligonucleotide agent.

Exemplary ligands are coupled, preferably covalently, either directly or indirectly via an intervening tether, to a ligand-conjugated carrier. In exemplary embodiments, the ligand is attached to the carrier via an intervening tether. In exemplary embodiments, a ligand alters the distribution, targeting or lifetime of an RNA silencing agent into which it is incorporated. In exemplary embodiments, a ligand provides an enhanced affinity for a selected target, e.g., molecule, cell or cell type, compartment, e.g., a cellular or organ compartment, tissue, organ or region of the body, as, e.g., compared to a species absent such a ligand.

Exemplary ligands can improve transport, hybridization, and specificity properties and may also improve nuclease resistance of the resultant natural or modified RNA silencing agent, or a polymeric molecule comprising any combination of monomers described herein and/or natural or modified ribonucleotides. Ligands in general can include therapeutic modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids, steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. Ligands can include a naturally occurring substance, (e.g., human serum albumin (HSA), low-density lipoprotein (LDL), or globulin); carbohydrate (e.g., a dextran, pullulan, chitin, chitosan, inulin, cyclodextrin or hyaluronic acid); amino acid, or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine, N-acetyl-glucosamine, multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. Other examples of ligands include dyes, intercalating agents (e.g. acridines and substituted acridines), cross-linkers (e.g. psoralene, mitomycin C), porphyrins (TPPC4, texaphyrin, Sapphyrin), polycyclic aromatic hydrocarbons (e.g., phenazine, dihydrophenazine, phenanthroline, pyrenes), lys-tyr-lys tripeptide, aminoglycosides, guanidium aminoglycodies, artificial endonucleases (e.g. EDTA), lipophilic molecules, e.g, cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C₁₀, C₁₁, C₁₂, C₁₃, C₁₄, C₁₅, C₁₆, C₁₇, C₁₈, C₁₉, or C₂₀ fatty acids) and ethers thereof, e.g., C₁₀, C₁₁, C₁₂, C₁₃, C₁₄, C₁₅, C₁₆, C₁₇, C₁₈, C₁₉, or C₂₀ alkyl; e.g., 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, stearic acid (e.g., glyceryl distearate), oleic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the RNA silencing agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The ligand can increase the uptake of the RNA silencing agent into the cell by activating an inflammatory response, for example. Exemplary ligands that would have such an effect include tumor necrosis factor alpha (TNFα), interleukin-1 beta, or gamma interferon. In one aspect, the ligand is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA. A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney. In a preferred embodiment, the lipid based ligand binds HSA. A lipid-based ligand can bind HSA with a sufficient affinity such that the conjugate will be preferably distributed to a non-kidney tissue. However, it is preferred that the affinity not be so strong that the HSA-ligand binding cannot be reversed. In another preferred embodiment, the lipid based ligand binds HSA weakly or not at all, such that the conjugate will be preferably distributed to the kidney. Other moieties that target to kidney cells can also be used in place of or in addition to the lipid based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low density lipoprotein (LDL).

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the RNA silencing agent, such as by enhancing cellular recognition and absorption. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. The peptide moiety can be an L-peptide or D-peptide. In another alternative, the peptide moiety can include a hydrophobic membrane translocation sequence (MTS). A peptide or peptidomimetic can be encoded by a random sequence of DNA, such as a peptide identified from a phage-display library, or one-bead-one-compound (OBOC) combinatorial library (Lam et al., Nature 354:82-84, 1991). In exemplary embodiments, the peptide or peptidomimetic tethered to an RNA silencing agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

### EXAMPLES

### Methods

All chemical reactions were performed under argon atmosphere using anhydrous freshly distilled solvents unless otherwise stated. Dichloromethane (DCM), acetonitrile (ACN) and dimethylformamide (DMF) were dried using a PureSolv MD 5× Channel Solvent Purification System, tested with Karl Fischer titration and stored on molecular sieves. Flash chromatography was performed using Teledyne Isco CombiFlash Rf system and prepacked (silica gel) columns purchased from Bonna-Agela Technologies (Tianjin, China). Analytical thin-layer chromatography (TLC) was performed using silica gel 60 F254 using UV light as visualizing agent. 1H, 13C and 31P NMR spectra were recorded on a Varian 400 MHz instruments using residual solvent or 85% phosphoric acid (for 31-P NMR) as reference. High-resolution mass spectra were obtained on an Agilent 6530 accurate-mass Q-TOF LC/MS (Agilent technologies, Santa Clara, CA).

### Example 1: Synthetic Approaches Used for Conjugation of Hydrophobic Compounds to Oligonucleotides

Using synthetic approaches outlined in FIGS. 1a-h, hsiRNAs covalently conjugated to cortisol, DHA, calciferol, cholesterol, and GM1 were synthesized. For cortisol (FIG. 1a) and calciferol (FIG. 1b), primary hydroxyls were converted to chloroformate and directly conjugated to the previously synthesized bi-functional, primary amine-containing, solid support. DHA was directly attached to the amino-modified linker using standard amide coupling conditions (FIG. 1c). GM1 was attached post-synthetically by click chemistry through the reaction of GM1-azide with alkyne modified siRNA (FIG. 1d and FIG. 1e). All compounds were HPLC-purified and characterized by mass spectrometry. The general synthesis strategies outlined in FIG. 1a-e are used to synthesize other related conjugates of Figure if Additional synthetic strategies are shown in FIG. 1g and FIG. 1h for the synthesis of calciferol conjugation, which may improve yields.

The oligonucleotide-conjugates were purified by reverse-phase HPLC, and the purity was assessed by liquid chromatography-mass spectrometry (LC-MS). Conditions: for analytical (FIG. 1i and FIG. 1j) (Anal HPLC: HTT-g2DHA-Cy3-P2, Pure product, Gradient: 10% MeCN, 90% TEAA to 90% MeCN, 10% TEAA in 30 minutes, Temp: room temperature, C8); for semi-preparative RP-HPLC (FIG. 1l) (Hamilton column, C18 HxSil 5 μm, 150×21.2 mm); for analytical RP-HPLC (FIG. 1m) (Agilent eclipse plus column, C18, 3.5 μm, 4.6×100 mm): Cy3-labeled sFLT-DHA conjugate (pure product), gradient: 10% acetonitrile, 90% TEAA to 90% acetonitrile, 10% TEAA in 30 minutes, Temperature: 60° C. (Analytical) and 55° C. (Preparative), flow rate: 20 mL/min (Preparative) and 1 ml/min (Analytical); for LC-MS (FIG. 1n) (Buffer A: 15 mM Dibutylamine/25 mM HFIP, Buffer B: 20% A in MeOH, Column: xbidge OST C18, 2.5 μm).

### Example 2: Structure and Hydrophobicity Profile of Selected Oligonucleotide Conjugates

To determine the relative hydrophobicity of a panel of novel conjugates, the retention time on a C8 reversed-phase HPLC column was measured. A higher hydrophobicity is correlated with longer retention times. FIG. 2b shows that the synthesized panel of conjugates encompasses a range of hydrophobicities: from cortisol (elution time of 4.5 min) to GM1 (elution time of 14 min).

All oligonucleotide conjugates were purified by reverse phase HPLC, and characterized by mass spectrometry (data for DHA-hsiRNA shown in FIG. 2c). The HPLC method was as follows: Reverse phase HPLC, C8; Buffer A: 100 mM NaAc and 5% acetonitrile, Buffer B: acetonitrile; Gradient: 5% B to 100% B over 15 minutes, 1.5 mL/min at 50° C.

### Example 3: In Vivo Brain Distribution of FMS-hsiRNA is Directly Related to Hydrophobicity

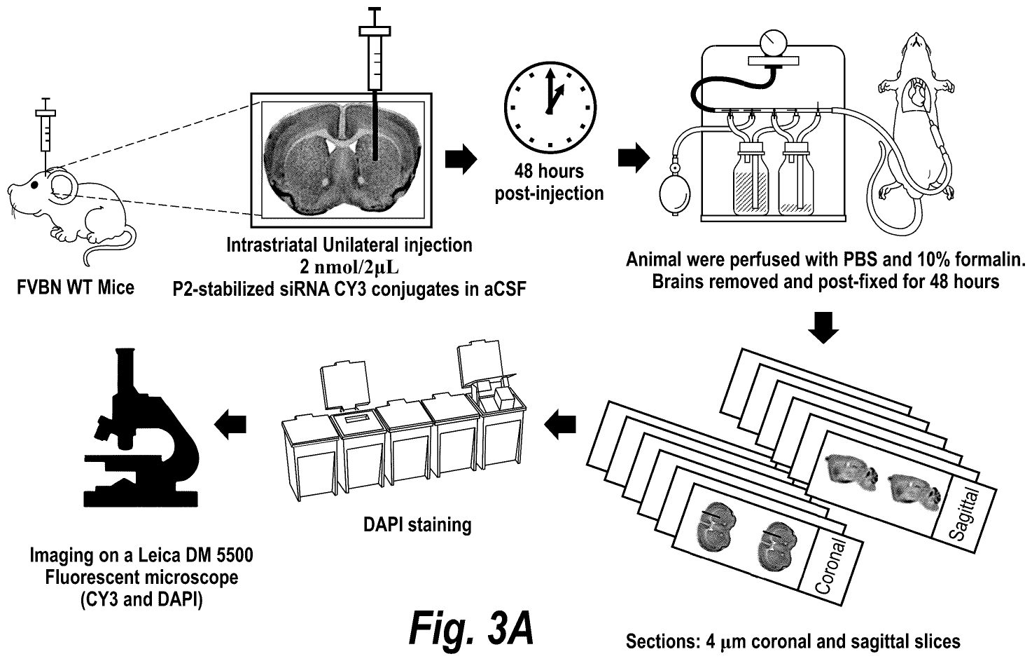

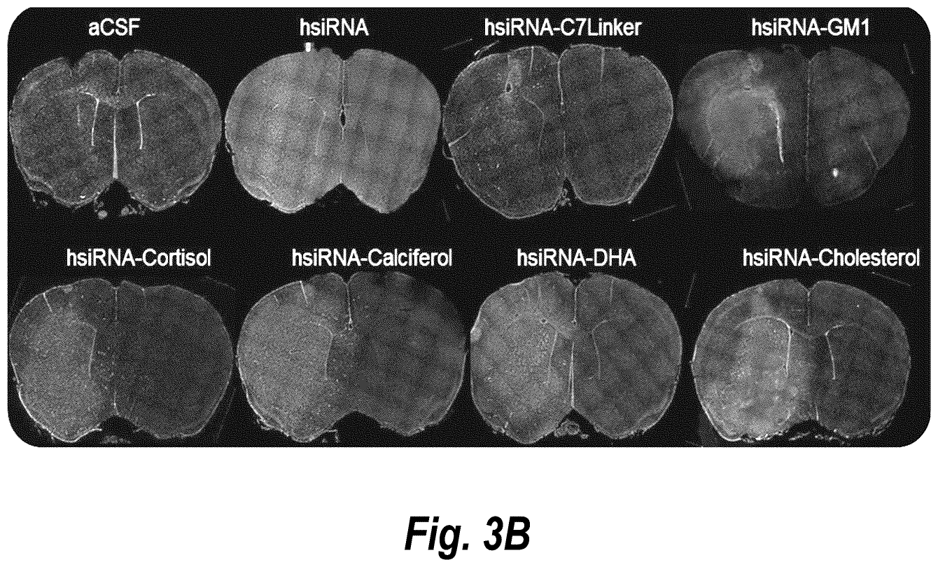

The present disclosure (FIG. 3a) shows that chemically modified and fully stabilized hydrophobic siRNA (hsiRNA) conjugates are successfully internalized by neurons and glia in the brain after intrastriatal administration (FIG. 3b). Furthermore, these data show a profound effect of conjugate chemistries on the pattern of in vivo brain distribution. The distribution of highly hydrophobic hsiRNA conjugates, including cholesterol- and GM1-, seem to be somewhat limited to the site of injection with very high intensity at this site. On the other hand, less hydrophobic hsiRNA conjugates, such as C7Linker- and TEGLinker-, show a more diffuse pattern with lower overall intensities. In addition, conjugates containing Calciferol- and DHA-show a distinct pattern of distribution characterized by a good spread throughout the section, which might be explained by potential receptor-mediated mechanism of uptake. Finally, it is also important to highlight that more hydrophobic hsiRNA conjugates, such as hsiRNA-GM1, hsiRNA-Calciferol, hsiRNA-DHA, and hsiRNA-cholesterol, enabled distribution to neuronal nerve bundles in the striatum. This may potentially result in retrograde axonal transport to the cortex.

To test the impact of hydrophobicity on tissue retention and brain distribution, 25 μg Cy3-labeled novel conjugates were injected unilaterally into striatum of wild-type mice and the fluorescence distribution was examined 48 hours later in both coronal and sagittal sections of the brain (FIG. 3b). Non-conjugated or linker-only hsiRNAs showed minimal but detectable retention in brain tissue. Importantly, it was found that the degree of tissue retention and distribution strongly correlates with hydrophobicity. Cortisol-hsiRNA (lowest degree of hydrophobicity) showed diffuse distribution, but the lowest tissue retention. The most hydrophobic compounds, cholesterol, and GM1, are effectively retained but do not distribute far from the site of injection. Tissue retention of FMS-hsiRNA was similar to that of LNA-gapmers, suggesting that the 13 phosphorothioate linkages in FMS-hsiRNA confer some level of tissue association. DHA and Calciferol hsiRNAs show optimal retention and spread throughout the injected side of the brain. The distribution of the calciferol-hsiRNA was so uniform, that it was impossible to map the site of injection, which is easily observed in animals injected with cholesterol or GM1 conjugates. In summary, it has been demonstrated that tuning the hydrophobicity of conjugates can be utilized to attain optimal retention and distribution in brain tissue.

As shown in the biodistribution study protocol of FIG. 3a, FVBN WT mice (n−3 per chemistry) were injected with 25 μg of Cy3-hsiRNA variants (P2-stabilized siRNA Cy3 conjugates in aCSF) via intrastitial unilateral injection (2 nmol/2 μL). After 48 hours, animals were perfused with PBS and 10% formalin. Brains were removed and post-fixed for 48 hours. 4 μm slices of coronal and sagittal sections were obtained, followed by DAPI staining. The samples were imaged (10×) on a Leica DM 5500 fluorescent microscope (Cy3 and DAPI); hsiRNA-FMS conjugates (Cy3—red), nuclei (DAPI—blue).

### Example 4: Systemic Delivery

Different hsiRNA variants were synthesized as described above and injected systemically (iv/sc) at 20 mg/kg. The level of accumulation of oligonucleotide in various tissues was determined by PNA Assay. The PNA (Peptide Nucleic Acid) hybridization assay directly measures an amount of intact guide strand in tissue lysates. This assay allows direct assessment of the rate of oligonucleotide clearance from CSF or blood as well as the degree of tissue distribution and accumulation (e.g., in different brain regions). This assay can detect both labeled and unlabeled compounds. Tissue accumulation of oligonucleotides above 10 ng/mg was sufficient to induce silencing.

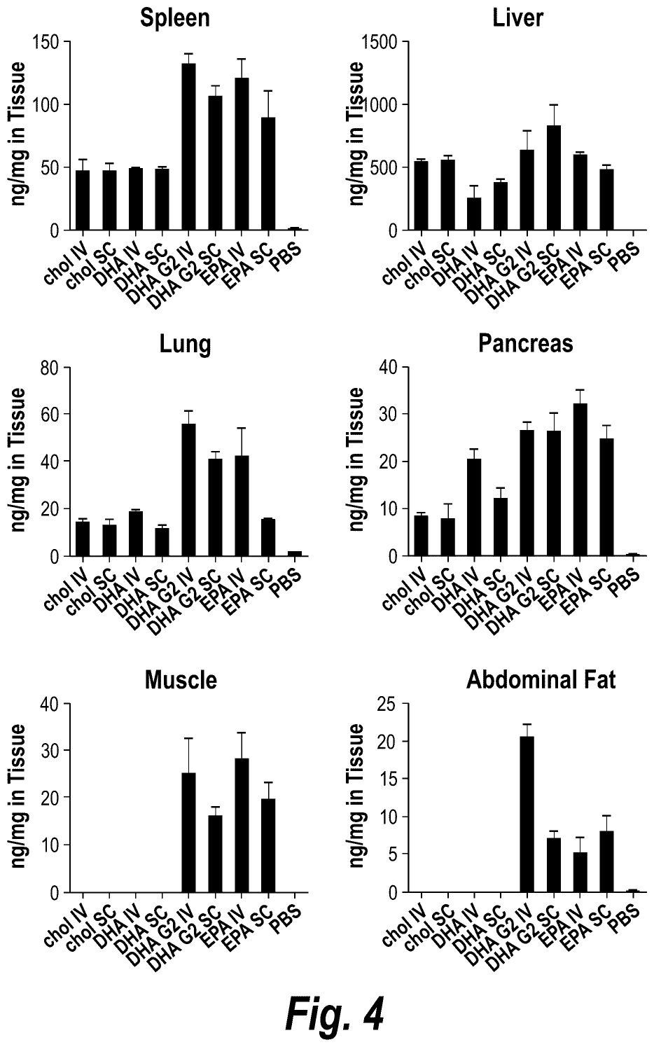

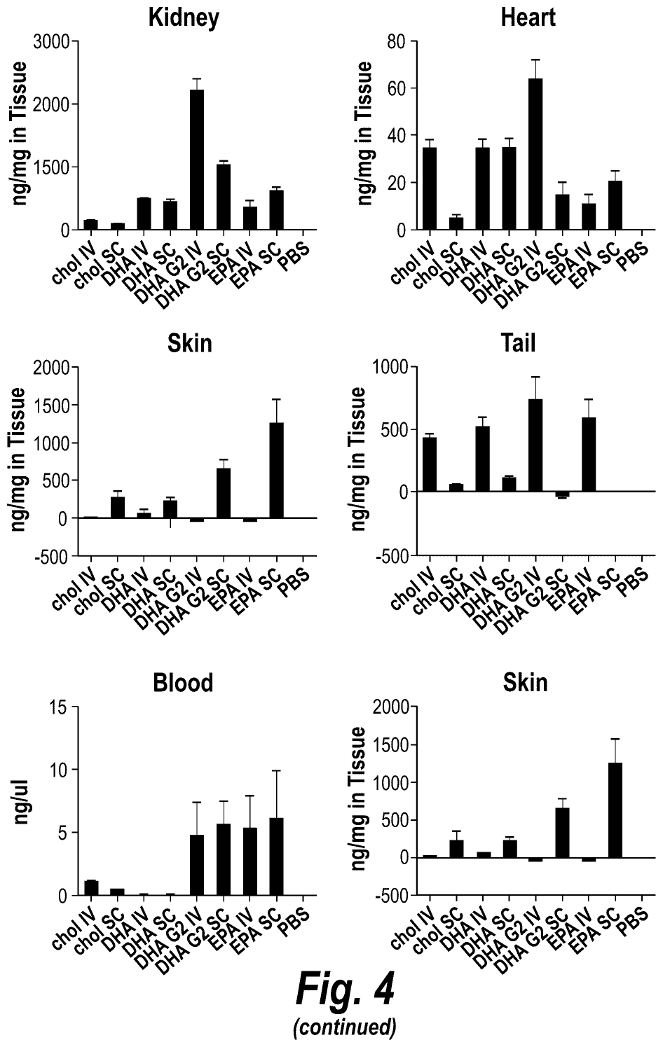

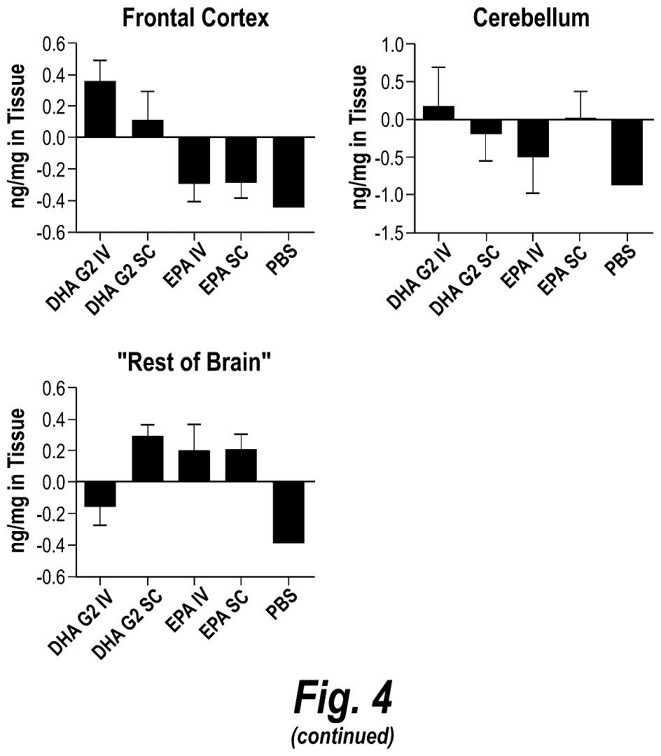

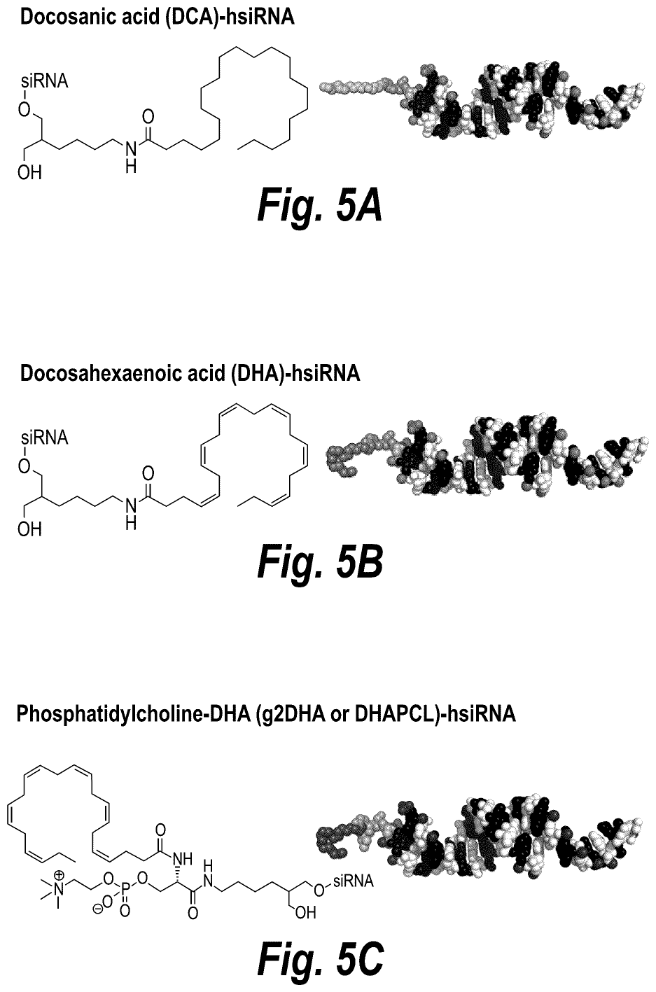

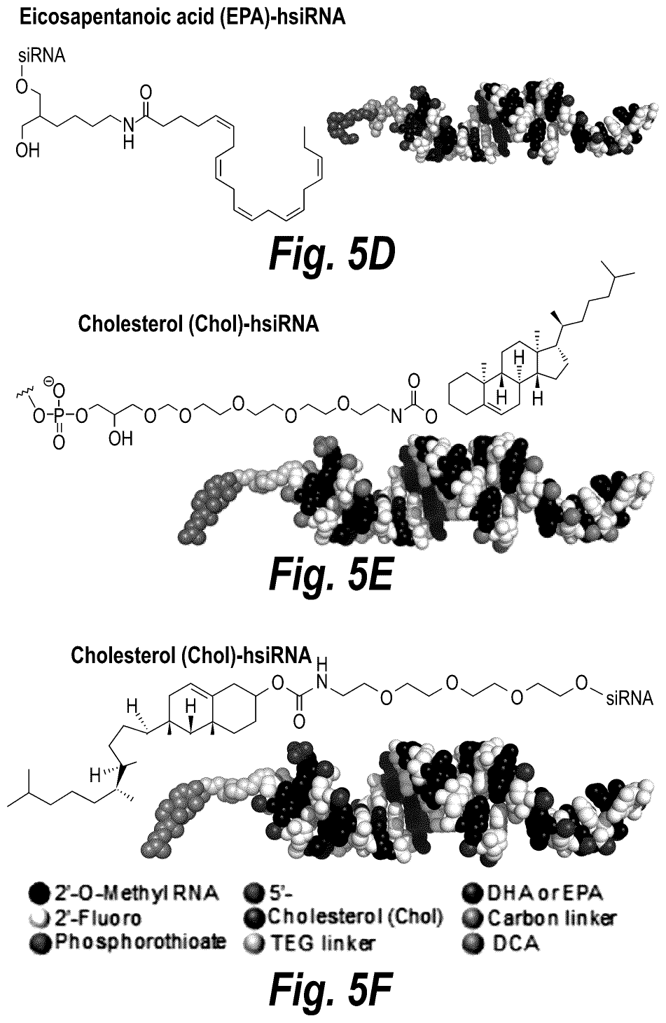

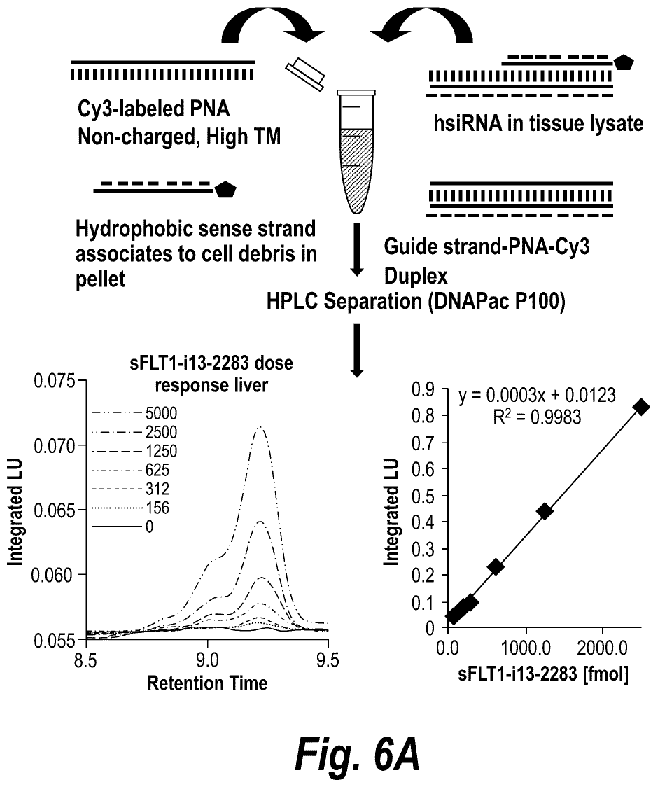

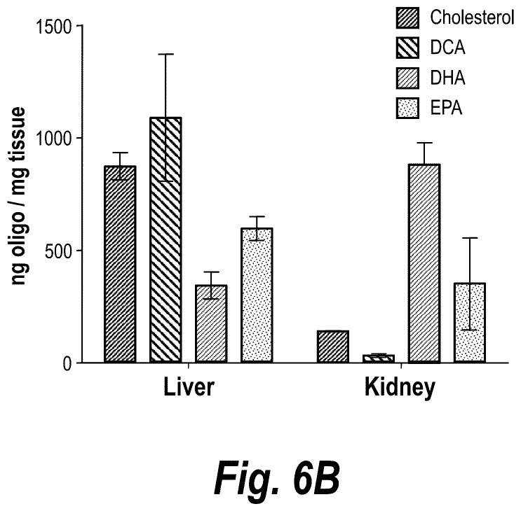

Surprisingly, different chemistries show preferential distribution to different tissues (FIG. 4). For example, PC-DHA shows accumulation in kidneys at above 2000 ng/mg levels and more compounds goes to kidney than to lung. Calciferol shows unprecedented distribution and preferential delivery to neurons in the brain. EPA shows the best skin distribution, relative to the compounds tested herein, where local injection delivers to a very wide area near the injection side.

### Example 5: g2DHA Support Synthesis II

As shown in FIG. 13, commercially available Fmoc-Ser(tBu)-OH is reacted with N,N-diisopropylamino methoxy phosphonamidic chloride to afford (1). (1) is then reacted with choline tosylate followed by oxidation with mCPBA to afford (2). Next, the Fmoc group on (2) is removed with 20% cyclohexylamine in DCM and the free amine is coupled to docosahexaenoic acid to afford (3) and (4) respectively. Following this, the tBu ester group on (4) is deprotected under acidic condition to yield (5). In a parallel line, the Fmoc group on a commercially available 1-O-DMT-6-N-Fmoc-2-hydroxymethylhexane support (6) is removed using a solution of 20% piperidine in dimethylformamide to produce (7). Finally, (5) and (7) are coupled in the presence of isobutyl chloroformate to yield the functionalized support (8).

CPG 8 (6.00 g, 330 μmol, 1 equiv.) was first treated with 20% piperidine in dry DMF for 15 minutes. This procedure was repeated twice to ensure complete deprotection of the Fmoc group. The amine-bearing CPG 9 was filtered off and washed successively with DCM, ACN and ether and dried under vacuum. Then the CPG 9 was mixed with a mixture of DHA (0.65 g, 1.98 mmol, 6 equiv.), HATU (0.25 g, 0.66 mmol, 2 equiv.) and DIEA (449 μL, 2.64 mmol, 8 equiv.) in dry DMF (42 mL). The suspension was mixed on a rotary mixer for 24 h. The CPG was then filtered off and washed with DCM, ACN and ether and dried under vacuum. The CPG was capped with 16% N-methylimidazole in THF (CAP A) and acetic anhydride:pyridine:THF (1:2:2, v/v/v) (CAP B) (1:1, v/v) during 15 min and was washed with DCM, ACN and ether and dried under vacuum.

### Example 6: Synthesis of DHAg2-hsiRNA from Functionalized Solid Support

### Preparation of Amine-Bearing CPG 3

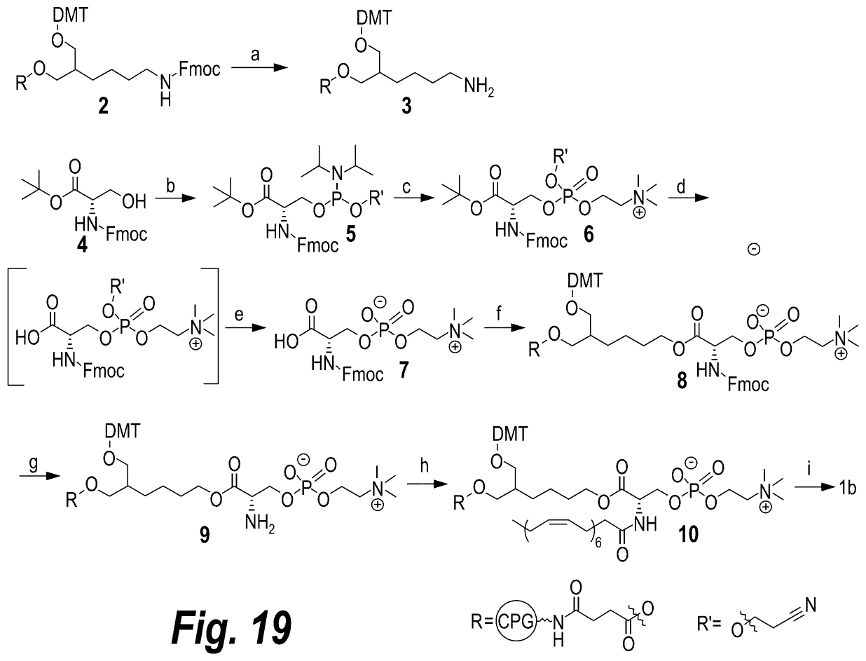

As shown in FIG. 19, a functionalized CPG (3, Scheme 2) was prepared and used for the solid-phase conjugation of DHA. First, the LCAA-CPG support (particle size 125-177 μm, pore diameter 500 Å and primary amino loading 145 μmol/g) was activated and dried overnight according to published protocols.¹ Then, the commercially available 1-O-DMT-6-N-Fmoc-2-hydroxymethylhexane was converted to succinate and loaded on CPG following a reported procedure to afford 2.² The linker loading was determined by DMT assay to be around 55 μmol/g. Subsequently, the Fmoc goup was removed from 2 using a solution of 20% piperidine in DMF for 15 minutes. This procedure was repeated twice to ensure complete deprotection of the Fmoc group. The amine-bearing CPG 3 was filtered off and washed successively with DCM, ACN and ether and dried under vacuum.

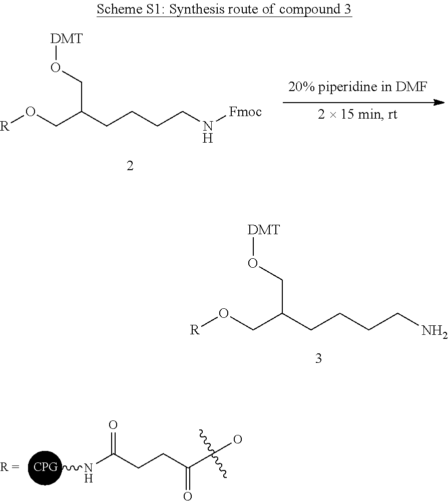

[1] M. J. Damha, P. A. Giannaris, S. V. Zabarylo, An improved procedure dor derivatization of controlled-pore glass beads for solid-phase oligonucleotide synthesis. Nucleic acids research 1990, 18, 3813-3821.
[2] P. S. Nelson, M. Kent, S. Muthini, Oligonucleotide labeling methods 3. Direct labeling of oligonucleotides employing a novel, non-nucleosidic, 2-aminobutyl-1,3-propanediol backbone. Nucleic acids research 1992, 20, 6253-6259.

### Synthesis of 5

Compound 4 (2.0 g, 5.21 mmol, 1 equiv.) was first dried by co-evaporation with toluene. Dry DCM (15 mL) and DIPEA (1.54 mL, 8.86 mmol, 1.7 equiv.) were added under argon and 2′-cyanoethyl-N,N-diisopropylchlorophosphoramidite (1.6 g, 6.78 mmol, 1.3 equiv.) was added slowly via a syringe. The reaction mixture was stirred 2 h at room temperature. After reaching completion, the reaction mixture was quenched with methanol and was washed with a solution of sodium bicarbonate and brine. The aqueous phase was extracted with DCM. The organic phase was dried on magnesium sulfate, filtrated and evaporated under vacuum. The crude was then purified by column chromatography on silica gel using a mixture of EtOAc/Hexane (8/2) with 1% pyridine as eluent, to afford 5 as a white solid (2.9 g, 4.97 mmol, yield 95%).

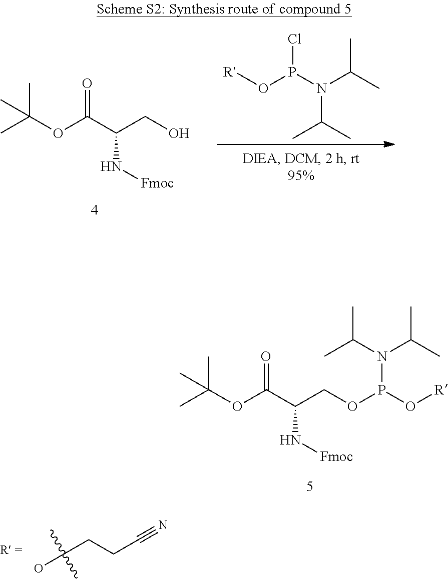

¹H NMR (400 MHz, CDCl₃) δ_{H} (ppm) 7.76 (d, J=7.6 Hz, 2H, Ar Fmoc); 7.62 (t, J=6.8 Hz, 2H, Ar Fmoc); 7.41 (t, J=7.6 Hz, 2H, Ar Fmoc); 7.32 (m, 2H, Ar Fmoc); 5.79-5.68 (dd, J=36.4 Hz, J=8.0 Hz, 1H, NH); 4.43-4.22 (m, 4H, CH₂ Fmoc+CH₂); 4.11-3.73 (m, 4H, 2*CH+CH₂ CE); 3.59 (m, 2H, 2*CH); 2.63-2.53 (m, 2H, CH₂ CE); 1.50, 1.49 (s, s, 9H, CH₃ tBu); 1.18 (m, 12H, CH₃). ¹³C NMR (100 MHz, CDCl₃) δ_{C} (ppm) 168.95 (C═O); 155.75 (C═O); 143.85, 143.70, 141.20, 141.18 (Cq Fmoc); 127.62, 126.99, 125.15, 125.09, 125.05, 125.03, 119.93, 119.80 (CH Ar Fmoc); 117.53 (Cq CE); 82.40 (Cq tBu); 67.08 (CH₂ Fmoc); 64.35 (CH₂); 63.93 (CH); 58.36 (CH₂ CE); 55.39 (CH); 47.07 (CH); 43.10 (CH Fmoc); 27.94 (CH₃ tBu); 24.56, 24.49 (CH₃); 20.30 (CH₂ CE). ³¹P NMR (161 MHz, CDCl₃) δ_{P} (ppm) 149.77, 149.74. HRMS (ESI⁻) m/z calculated for C₃₁H₄₂N₃O₆P (M+Na) 605.2708; Found 605.2306.

### Synthesis of 6

Compound 5 (2.9 g, 5.39 mmol, 1 equiv.) was dried with dry toluene and dry ACN. Choline p-toluenesulfonate (1.63 g, 5.93 mmol, 1.1 equiv.) was dried with toluene and dissolved in dry ACN (46 mL). This mixture was added to compound 5 through a cannula. ETT (0.25 M in ACN) (21.6 mL, 5.39 mmol, 1 equiv.) was added slowly with a syringe. The mixture was stirred 2 h at room temperature. After reaching completion, the reaction mixture was quenched with methanol. Meta-chloroperoxybenzoic acid (mCPBA) (1.86 g, 10.78 mmol, 2 equiv.) was added by portion to the mixture. After 30 min of stirring, the mixture was reduced under vacuum. The crude was then purified by column chromatography on silica gel using a gradient of MeOH in DCM (0-30%) as eluent, to afford 6 as a mixture of tetrazolium (major counter anion) and tosylate (less than 5%) salts (2.7 g, 3.69 mmol, yield 69%).

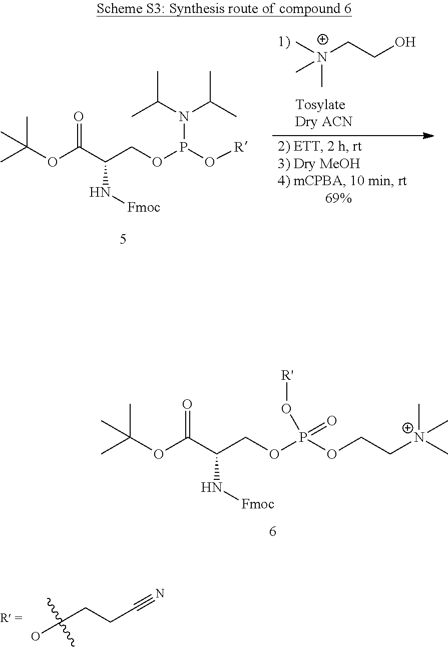

¹H NMR (400 MHz, CDCl₃) δ_{H} (ppm) 7.72 (d, J=7.6 Hz, 2H, Ar Fmoc); 7.66 (d, J=8.0 Hz, 2H, Ar tosylate); 7.59 (d, J=7.2 Hz, 2H, Ar Fmoc); 7.36 (t, J=7.2 Hz, 2H, Ar Fmoc); 7.27 (t, J=8.0 Hz, 2H, Ar Fmoc); 7.09 (d, J=8.0 Hz, 2H, Ar tosylate); 6.80-6.70 (dd, J=33.2 Hz, J=7.2 Hz, 1H, NH); 4.51-4.36 (m, 6H, CH₂ Fmoc+2*CH₂); 4.29-4.15 (m, 4H, CH₂ CE+2*CH); 3.83 (m, 2H, CH₂); 3.25 (q, J=7.2 Hz, 2H, CH₂ tetrazolium); 3.19 (s, 9H, CH₃); 2.72 (m, 2H, CH₂ CE); 2.27 (s, 3H, CH₃ tosylate); 1.44 (s, 9H, CH₃ tBu); 1.18 (t, J=7.2 Hz, 3H, CH₃ tetrazolium). ¹³C NMR (100 MHz, CDCl₃) δ_{C} (ppm) 167.77 (C═O); 163.89 (Cq tetrazolium); 156.16 (C═O); 143.69, 143.63, 141.11 (Cq Fmoc); 128.81, 125.63 (CH tosylate); 127.69, 127.07, 125.24, 125.17, 119.91, (CH Ar Fmoc); 143.15, 139.73 (Cq tosylate); 117.18 (Cq CE); 83.22 (Cq tBu); 67.96 (CH₂); 67.14 (CH₂ Fmoc); 65.25 (CH₂); 62.91 (CH₂ CE); 61.88 (CH); 54.85 (CH₂); 54.10 (CH₃); 46.88 (CH Fmoc); 27.86 (CH₃ tBu); 21.18 (CH₃ tosylate); 19.58 (CH₂ tetrazolium); 19.51 (CH₂ CE); 6.80 (CH₃ tetrazolium). ³¹P NMR (161 MHz, CDCl₃) δ_{P} (ppm) −2.60, −2.71. HRMS (ESI⁺) m/z for calculated C₃₀H₄₁N₃O₈P (M+H) 603.2799; Found 603.2853.

Note: The order of addition of reactants during the synthesis of 6 is important. If compound 5 and ETT are mixed prior to the addition of choline p-toluenesulfonate a side reaction will occur according to the Scheme S4.

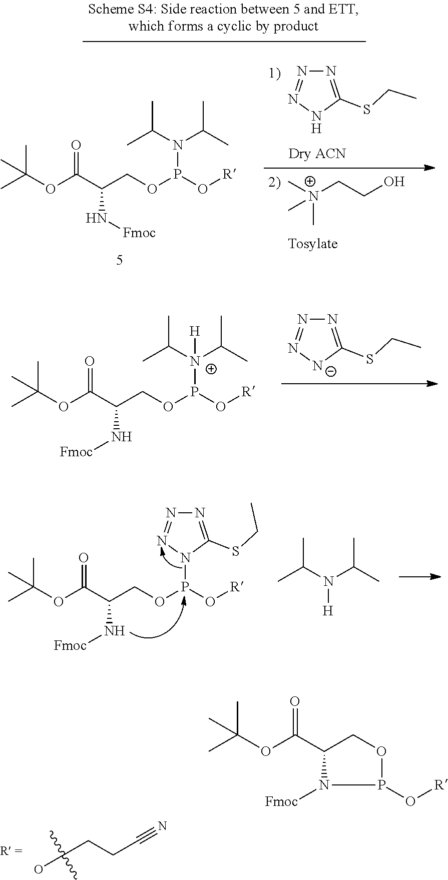

### Synthesis of 7

Compound 6 (2.30 g, 3.15 mmol, 1 equiv.) was dissolved in 60 mL of (1:1) solution of TFA:dry DCM. Triisopropylsilane (2.39 mL, 11.66 mmol, 3.7 equiv.) was added and the mixture was stirred at room temperature for 2 h. The solvent and TFA were evaporated and the residue was purified by reverse phase HPLC (C₁₈, Buffer A=Water, Buffer B=ACN, Gradient=5-65% of B in 12 min, T=45° C.). The ACN was removed under vacuum and the aqueous solution was freeze-dried. The lyophilized powder was dissolved in 10% diisopropylethylamine (14 mL) in ACN (140 mL) and the mixture was stirred at room temperature for 2 h. The solvent was evaporated under vacuum and the crude was purified by reverse phase HPLC (C₁₈, Buffer A=Water, Buffer B=ACN, Gradient=5-65% of B in 12 min, T=45° C.). The ACN was removed under vacuum and the aqueous solution was freeze-dried to afford 7 as diisopropylammonium salt (1.38 g, 2.32 mmol, yield 74% over two steps).

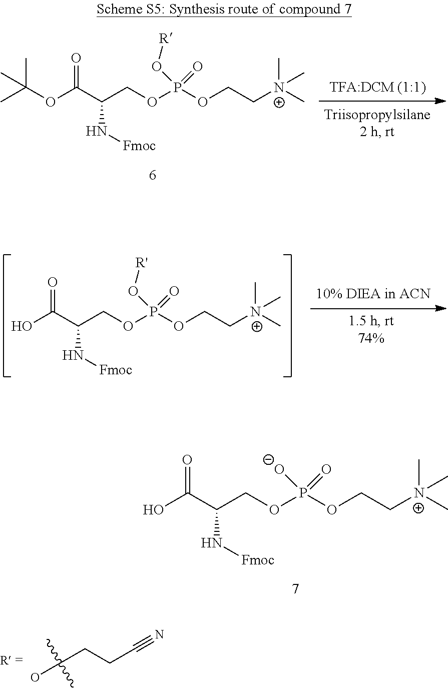

¹H NMR (400 MHz, DMSO-d6) δ_{H} (ppm) 7.88 (d, J=7.5 Hz, 2H, Ar Fmoc); 7.85-7.70 (m, 2H, Ar Fmoc); 7.41 (t, J=7.0 Hz, 2H, Ar Fmoc); 7.34 (t, J=7.0 Hz, 2H, Ar Fmoc); 6.75 (s, 1H NH); 7.28 (s, 1H NH); 4.26-4.04 (m, 5H, CH₂+CH Fmoc+CH₂ Fmoc); 3.92 (s, 2H, CH₂); 3.78-3.38 (m, 5H, CH+CH₂+2*CH DIPEA); 3.13 (s, 9H, CH₃); 1.14, 1.12 (s,s, 12H, CH₃ DIPEA). ¹³C NMR (100 MHz, DMSO-d6) δ_{C} (ppm) 170.94 (C═O); 155.13 (C═O); 143.90, 142.46, 140.57, 139.31 (Cq Fmoc); 137.32, 128.81, 127.48, 127.18, 125.11, 121.27, 119.92, 109.64 (CH Ar Fmoc); 65.39 (CH₂); 65.24 (CH₂ Fmoc); 65.15 (CH); 58.21 (CH₂); 56.78 (CH₂); 52.89 (CH₃); 46.61 (CH Fmoc); 45.12 (CH DIPEA); 19.78 (CH₃ DIPEA). ³¹P NMR (161 MHz, CDCl₃) δ_{P} (ppm) −1.15 HRMS (ESI⁺) m/z for calculated C₂₃H₂₉N₂O₈P (M+H) 493.1788; Found 493.1783.

### Solid-Phase Synthesis of 8

Compound 7 (1.00 g, 1.69 mmol, 4.75 equiv.) was dissolved in dry DMF (100 mL). (Benzotriazol-1-yloxy)tris(dimethylamino)phosphonium hexafluorophosphate (BOP) (0.59 g, 1.34 mmol, 3.76 equiv.) and hydroxybenzotriazol (HOBt) (0.21 g, 1.34 mmol, 3.76 equiv.) were added and stirred until the solution went clear. 2,4,6-collidine (560 μL, 4.32 mmol, 12.42 equiv.) was added followed by 3 (6.55 g, loading of 55 μmol/g, 360 μmol, 1 equiv.) and the suspension was mixed overnight on a rotary mixer. The CPG was filtered off and washed with DCM, ACN and ether and dried under vacuum. The CPG was capped with 16% N-methylimidazole in THF (CAP A) and acetic anhydride:pyridine:THF (1:2:2, v/v/v) (CAP B) (1:1, v/v) for 1 h and was washed with DCM, ACN and ether and dried under vacuum.

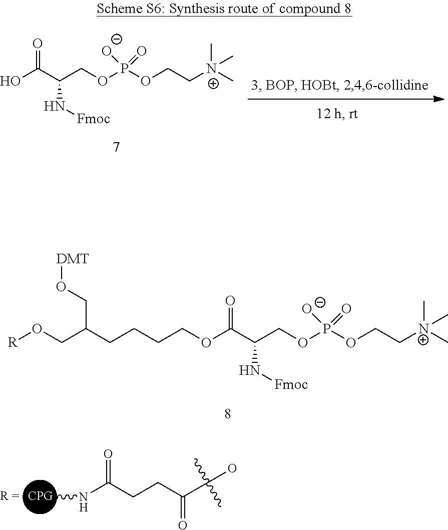

### Solid-Phase Synthesis of 9 and 10

CPG 8 (6.00 g, 330 μmol, 1 equiv.) was first treated with 20% piperidine in dry DMF for 15 minutes. This procedure was repeated twice to ensure complete deprotection of the Fmoc group. The amine-bearing CPG 9 was filtered off and washed successively with DCM, ACN and ether and dried under vacuum. Then the CPG 9 was mixed with a mixture of DHA (0.65 g, 1.98 mmol, 6 equiv.), HATU (0.25 g, 0.66 mmol, 2 equiv.) and DIEA (449 μL, 2.64 mmol, 8 equiv.) in dry DMF (42 mL). The suspension was mixed on a rotary mixer for 24 h. The CPG was then filtered off and washed with DCM, ACN and ether and dried under vacuum. The CPG was capped with 16% N-methylimidazole in THF (CAP A) and acetic anhydride:pyridine:THF (1:2:2, v/v/v) (CAP B) (1:1, v/v) during 15 min and was washed with DCM, ACN and ether and dried under vacuum.

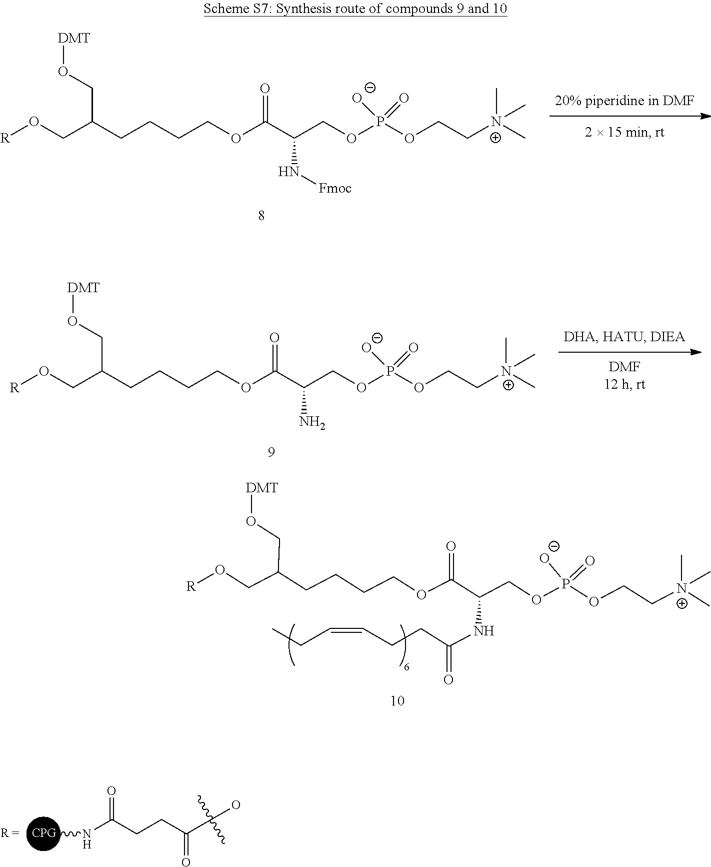

### Standard Solid-Phase Oligonucleotide Synthesis

Oligonucleotides were synthesized on an Expedite ABI DNA/RNA Synthesizer following standard protocols. Each synthesis was done at a 1-μmole scale using DHA-conjugated CPG 10 for the sense strand and a Unylinker® terminus (ChemGenes, Wilmington, MA) for the antisense strand. Phosphoramidites were prepared as 0.15 M solutions for 2′-O-methyl (ChemGenes, Wilmington, MA) and Cy3 (Gene Pharma, Shanghai, China) and 0.13 M for 2′-fluoro (BioAutomation, Irving, Texas) in ACN. 5-(Benzylthio)-1H-tetrazole (BTT) 0.25 M in ACN was used as coupling activator. Detritylations were performed using 3% dichloroacetic acid (DCA) in DCM for 80 s and capping was done with a 16% N-methylimidazole in THF (CAP A) and THF:acetic anhydride:2,6-lutidine, (80:10:10, v/v/v) (CAP B) for 15 s. Sulfurizations were carried out with 0.1 M solution of DDTT in ACN for 3 minutes. Oxidation was performed using 0.02 M iodine in THF:pyridine:water (70:20:10, v/v/v) for 80 s. Phosphoramidite coupling times were 250 s for all amidites.

### Deprotection and Purification of Oligonucleotides

Both sense and antisense strands were cleaved and deprotected using 1 mL of 40% aq. methylamine at 65° C. for 10 minutes. The oligonucleotide solutions were then cooled in a freezer for a few minutes and dried under vacuum in a Speedvac. The resulting pellets were suspended in 10 mL of triethylammonium acetate (TEAA) buffer (0.1 M, pH 7) and filtered through a 0.2 μm filter. The final purification of oligonucleotides was performed on an Agilent Prostar System (Agilent, Santa Clara, CA) equipped with a Hamilton HxSil C8 column (150×21.2) using the following conditions: buffer A: (0.1 M, TEAA, PH 7), B: (ACN), gradient: 90% A, 10% B to 10% A, 90% B in 30 minutes, temperature: 55° C., flow rate: 20 ml/min. The pure oligonucleotides were collected and cation-exchanged on a HiTrap 5 ml SP HP column (GE Healthcare Life Sciences, Marlborough, MA) and lyophilized.

### Example 7: Solid Phase Synthesis of DHAg2-hsiRNA

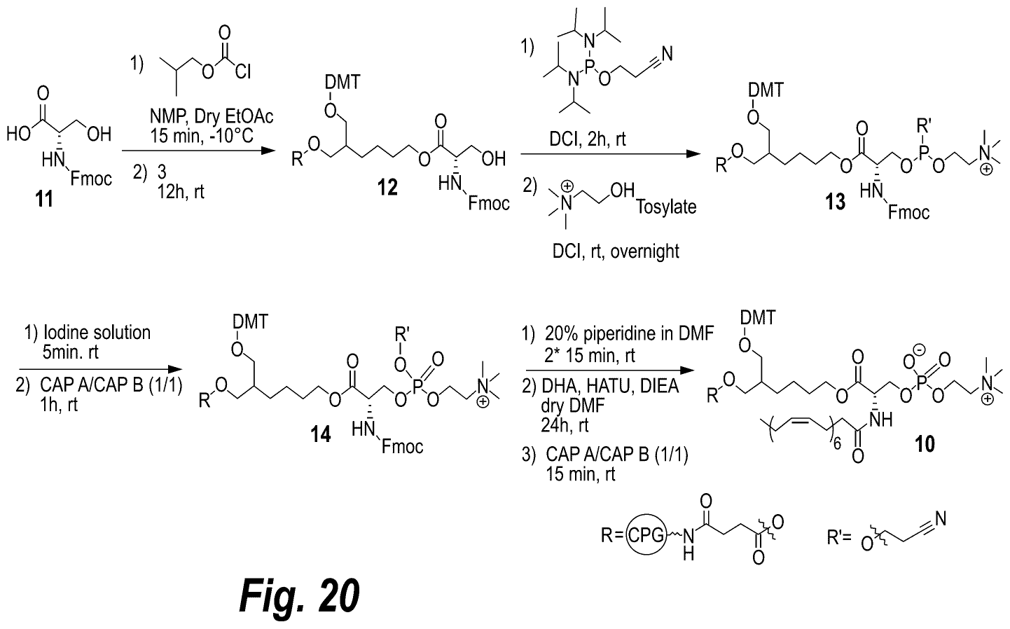

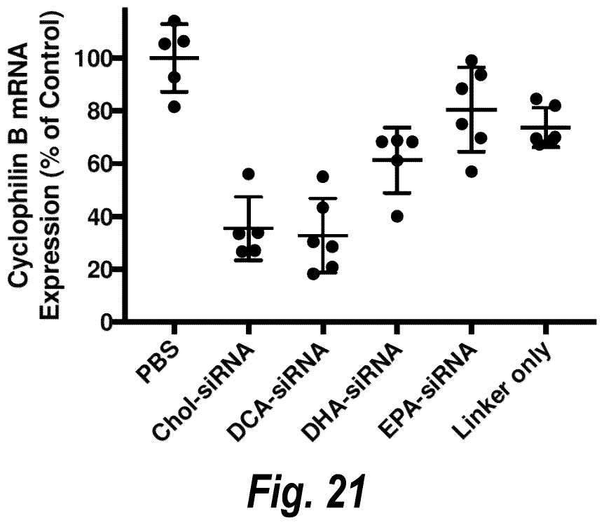

As shown in FIG. 20, the commercially available N-Fmoc-L-serine 11 (0.38 g, 1.14 mmol) was placed in a round bottom flask and dried by co-evaporation with toluene. Anhydrous ethyl acetate (3 mL) was delivered to the flask and the solution was cooled down to −10° C. Isobutyl chloroformate (0.15 mL, 0.16 g, 1.16 mmol) and N-methyl-2-pyrrolidone (NMP) (0.26 mL, 2.65 mmol) were added to this solution and the mixture was stirred for 15 minutes. Linker 3 (0.08 mmol) was added under argon and the suspension was mixed on a rotary mixer for 12 h. The CPG was filtered off and washed with DCM, ACN and ether and dried under vacuum to afford 12. 12 was placed in a small peptide synthesis flask and rinsed twice with dry ACN and kept under argon. 2-cyanoethyl-N,N,N′N′-tetraisopropylphosphorodiamidite (0.61 mL, 1.91 mmol) and 4,5-dicyanoimmidazole (DCI) (7.65 mL of a 0.25 M solution in ACN, 1.91 mmol) were added and the suspension was mixed on a rotary mixer for 2 h. The solution was decanted and the CPG was kept under argon. Choline p-toluenesulfonate (0.53 g, 1.91 mmol) that was previously dried by co-evaporation with toluene was mixed with 4,5-dicyanoimmidazole (DCI) (7.65 mL of a 0.25 M solution in ACN, 1.91 mmol) and delivered to the flask via a syringe. The suspension was mixed on a rotary mixer overnight. The solution was decanted and the CPG was washed with dry acetonitrile to afford CPG 13. Subsequently, the phosphotriester group was oxidized with iodine solution (7.6 mL of a 0.02 M iodine in THF:pyridine:water 70:20:10, v/v/v, 0.15 mmol) for 5 minutes and capped with a mixture (1/1, v/v) of 16% N-methylimidazole in THF (CAP A) and THF:acetic anhydride:2,6-lutidine, (80:10:10, v/v/v) (CAP B) for 1 h. The CPG was washed with DCM, ACN and ether and dried under vacuum to yield 14. The Fmoc group of 14 was then removed by treating the CPG with 20% piperidine in DMF (2×15 minutes). Piperidine simultaneously removes the O-cyanoethyl protecting group generating a phosphodiester specie. The CPG was washed and dried again as previously described. The amine-bearing CPG was then added to a mixture of DHA (0.19 g, 0.20 mL, 0.568 mmol), HATU (0.07 g, 0.18 mmol, and DIEA (0.39 mL, 2.24 mL) in dry DMF and stirred overnight. The solution was decanted and the CPG was capped with 16% N-methylimidazole in THF (CAP A) and acetic anhydride:pyridine: THF (1:2:2, v/v/v) (CAP B) (1:1, v/v) for 30 minutes. Finally, the CPG was washed with DCM, ACN and ether and dried under vacuum to afford 10.

### Example 8: In Vivo Assays of Various Conjugates

### Microscopy

Wild-type (FVBN/J) mice (female, 6-7-week-old, n=3 per conjugate) were injected subcutaneous with Cy3-HTT hsiRNA duplexes (20 mg/kg). After 48 h, the mice were euthanized, perfused with 1×PBS and the organs were collected and let in 10% formalin overnight at 4 C. The tissues were post-fixed, paraffin-embedded, and sliced into 4 μm sections. Each tissue section was mounted on a glass slide and stained with DAPI to visualize nuclei prior to image collection on a Leica DMi8 inverted microscope with 5× objective. The cy3 channel of all organs are represented.

DHA g1 was observed by microscopy (5×) in the thymus, bladder, intestine, skin, bone marrow, placenta, adipose, spleen, liver and kidney. DHA g2 was observed by microscopy (5×) in the thymus, bladder, intestine, skin, bone marrow, placenta, adipose, muscle, spleen, pancreas, liver and kidney.

DCA g1 was observed by microscopy (5×) in the skin, bone marrow, placenta, adipose, spleen, lung, adrenal gland, heart, kidney and liver. DCA g2 was observed by microscopy (5×) in the thymus, skin, bone marrow, placenta, adipose, muscle, spleen, pancreas, lung, fallopian tube, adrenal gland, heart, kidney and liver.

EPA g1 was observed by microscopy (5×) in the bladder, intestine, skin, bone marrow, adipose, muscle, spleen, pancreas, lung, heart, kidney and liver. EPA g2 was observed by microscopy (5×) in the intestine, skin, bone marrow, muscle, spleen, lung, fallopian tube, heart, kidney and liver.

Cholesterol g1 was observed by microscopy (5×) in the placenta, spleen, adrenal gland, heart, kidney and liver. Cholesterol g2 was observed by microscopy (5×) in the bone marrow, spleen, adrenal gland, heart, kidney and liver.

LA g1 was observed by microscopy (5×) in the bladder, skin, bone marrow, adipose, spleen, pancreas, adrenal gland, kidney and liver. LA g2 was observed by microscopy (5×) in the bladder, intestine, skin, bone marrow, adipose, spleen, fallopian tube, adrenal gland and kidney.

RA g1 was observed by microscopy (5×) in the thymus, intestine, skin, bone marrow, adipose, muscle, spleen, lung, fallopian tube, adrenal gland, kidney and liver. RA g2 was observed by microscopy (5×) in the thymus, skin, bone marrow, spleen, pancreas, lung, adrenal gland, kidney and liver.

TOCO g1 was observed by microscopy (5×) in the bone marrow, muscle, spleen, fallopian tube kidney and liver. TOCO g2 was observed by microscopy (5×) in the spleen and liver.

Choline was observed by microscopy (5×) in the placenta, kidney and bone marrow.

### PNA Hybridization Assay

Levels of hsiRNA guide (antisense) strand accumulation in tissues were quantified using a PNA hybridization assay. Tissue punches were homogenized in MasterPure Tissue Lysis Solution (EpiCentre) with added proteinase K (2 mg/mL, Invitrogen) and homogenized using a TissueLyser II (Qiagen), using 100 μL of lysis solution per 10 mg tissue. Following lysis, sodium dodecyl sulfate was precipitated with KCl (3 mol/l) and cleared supernatant was hybridized to a Cy3-labeled PNA oligonucleotide fully complementary to the guide strand (PNABio). This mixture was analyzed by high-performance liquid chromatography. Cy3-labeled peaks were integrated and plotted on an internal calibration curve. Mobile phase for HPLC was 50% water, 50% acetonitrile, 25 mmol/1 Tris-HCl (pH 8.5) and 1 mmol/l ethylenediamine-tetraacetate. The salt gradient was 0-800 mmol/1 NaClO₄.

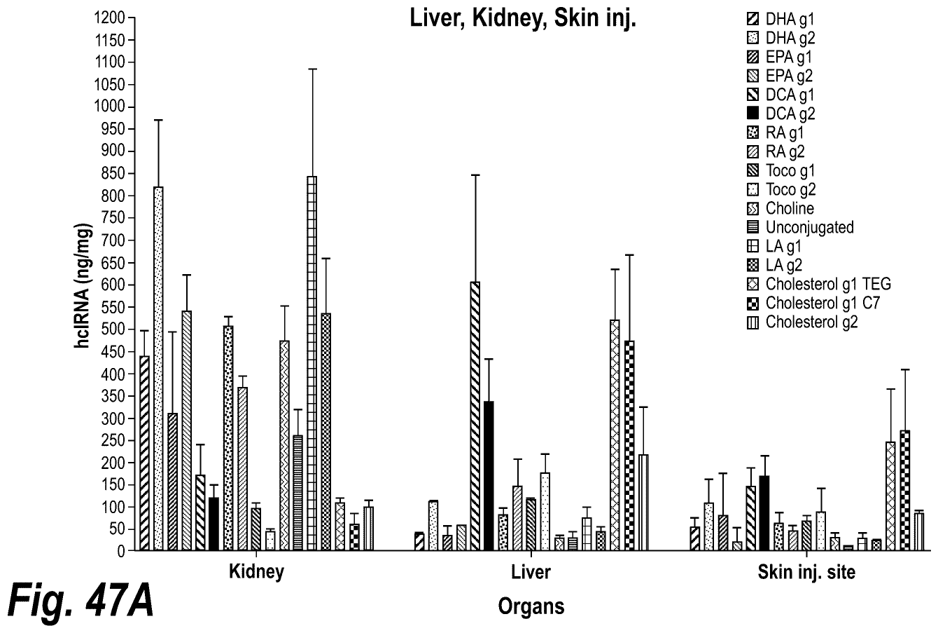

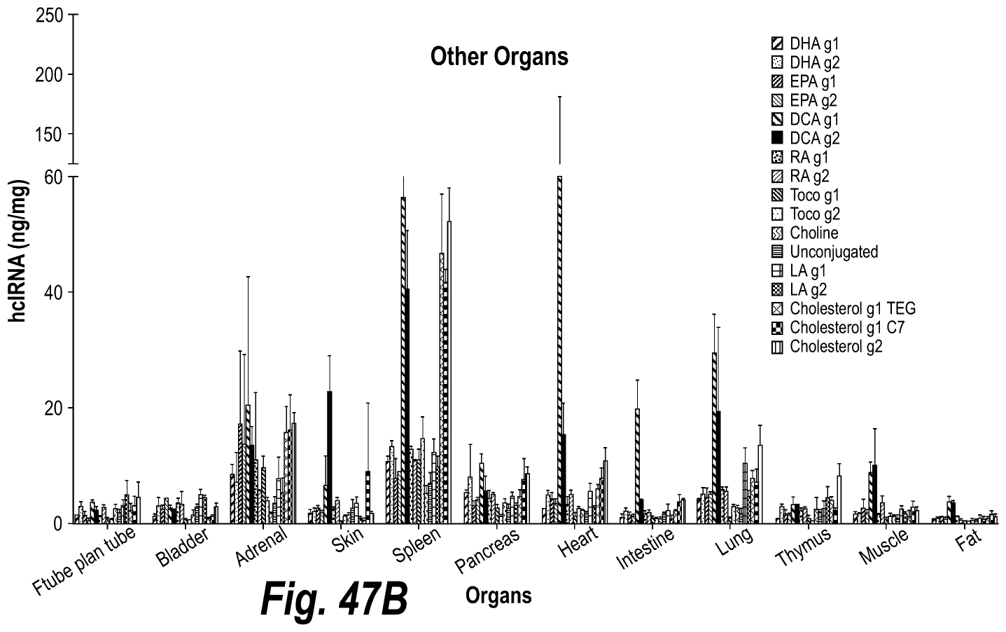

Results of the PNA assays are shown in FIGS. 47A-B, which depict delivery of each of DHA g1, DHA g2, EPA g1, EPA g2, DCA g1, DCA g2, RA g1, RA g2, TOCO g1, TOCO g2, choline, LA g1, LA g2, cholesterol g1, cholesterol C7 g1 and cholesterol g2 in the kidney, liver, skin at the injection site, skin distal to the injection site, fallopian tube, bladder, adrenal gland, spleen, pancreas, intestine, lung, thymus, muscle and adipose tissue.

### Efficacy Studies

Wild-type (FVBN/J) mice (female, 6-7 week-old, n=8 per conjugate) were injected subcutaneous with Cy3-HTT hsiRNA or Cy3-PPIB hsiRNA duplexes (20 mg/kg). After 1 week, the mice were euthanized, perfused with 1×PBS and the organs were placed in RNA later overnight at 4 C and at −80 C. Punches were processed and mRNA levels were measured using QuantiGene® 2.0 DNA Assay, normalized to housekeeping gene (HPRT) and presented as % of PBS control (+/−SD). Data were analyzed using GraphPad Prism 6 software using one-way ANOVA comparison.

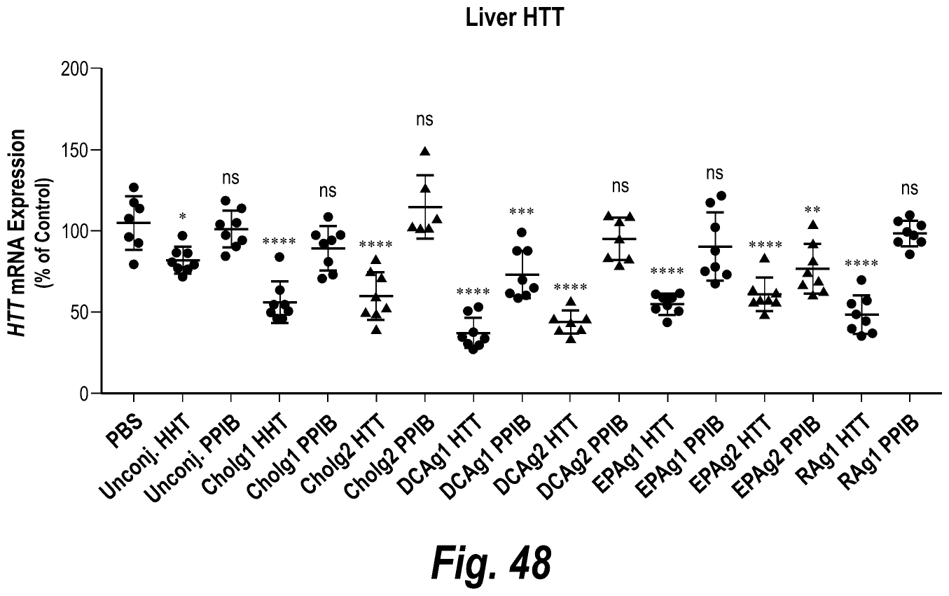

The results of such efficacy studies are set forth in FIGS. 48-60.

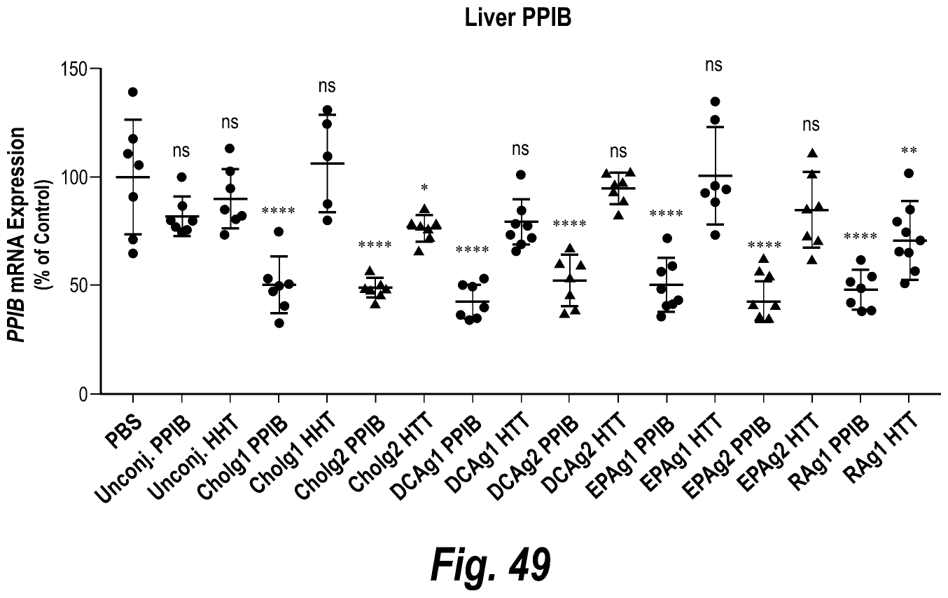

FIG. 48 depicts efficacy of unconjugated htt hsiRNA, cholesterol g1-congugated htt hsiRNA, cholesterol g2-conjugated htt hsiRNA, DCA g1-conjugated htt hsiRNA, DCA g2-conjugated htt hsiRNA, EPA g1-conjugated htt hsiRNA, EPA g2-conjugated htt hsiRNA and RA g1-conjugated htt hsiRNA for silencing htt mRNA in the liver. FIG. 49 depicts efficacy of cholesterol g1-congugated PPIB hsiRNA, cholesterol g2-conjugated PPIB hsiRNA, DCA g1-conjugated PPIB hsiRNA, DCA g2-conjugated PPIB hsiRNA, EPA g1-conjugated PPIB hsiRNA, EPA g2-conjugated PPIB hsiRNA and RA g1-conjugated PPIB hsiRNA for silencing PPIB mRNA in the liver.

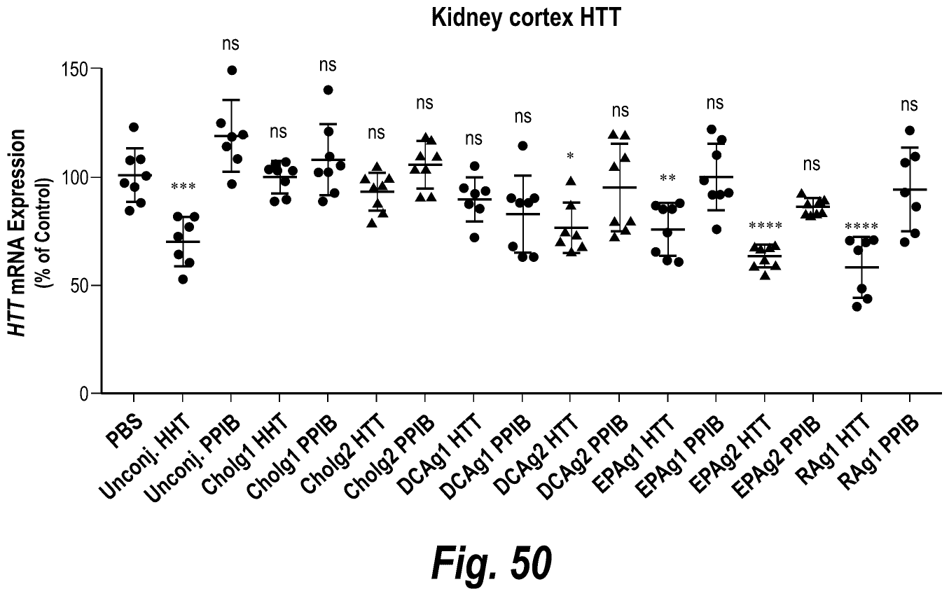

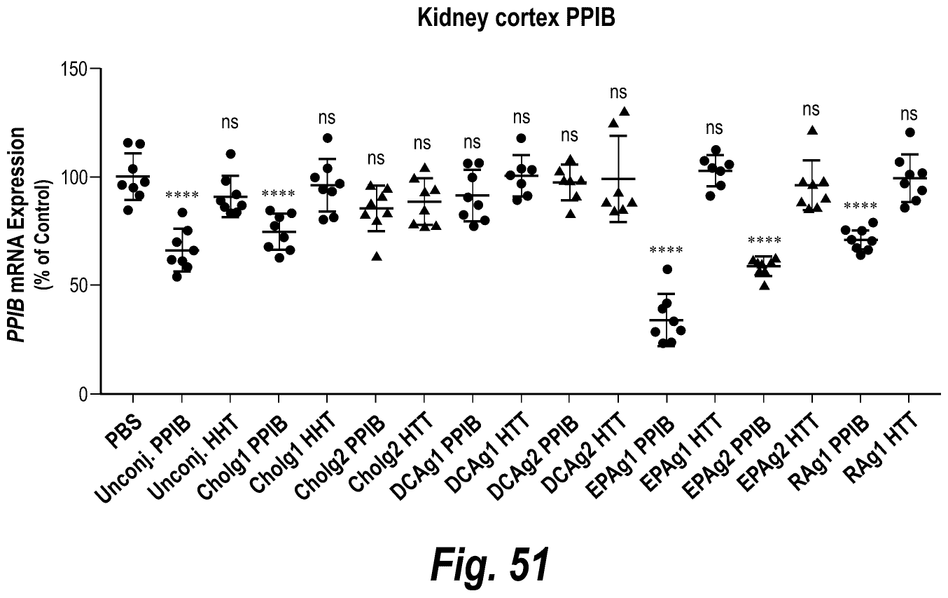

FIG. 50 depicts efficacy of unconjugated htt hsiRNA, DCA g2-conjugated htt hsiRNA, EPA g1-conjugated htt hsiRNA, EPA g2-conjugated htt hsiRNA and RA g1-conjugated htt hsiRNA for silencing htt mRNA in kidney cortex. FIG. 51 depicts efficacy of unconjugated PPIB hsiRNA, cholesterol g1-conjugated PPIB hsiRNA, EPA g1-conjugated PPIB hsiRNA, EPA g2-conjugated PPIB hsiRNA and RA g1-conjugated PPIB hsiRNA for silencing PPIB mRNA in kidney cortex.

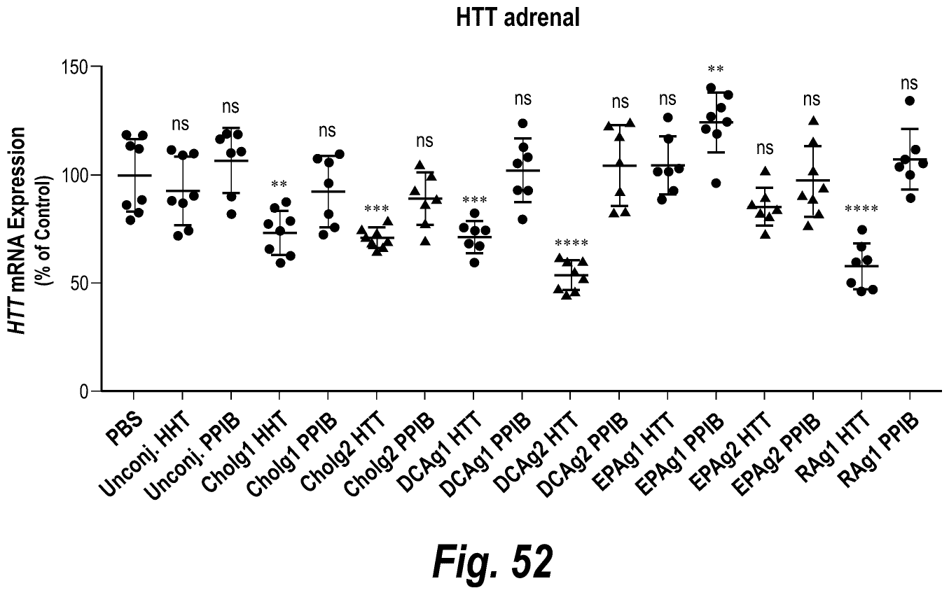

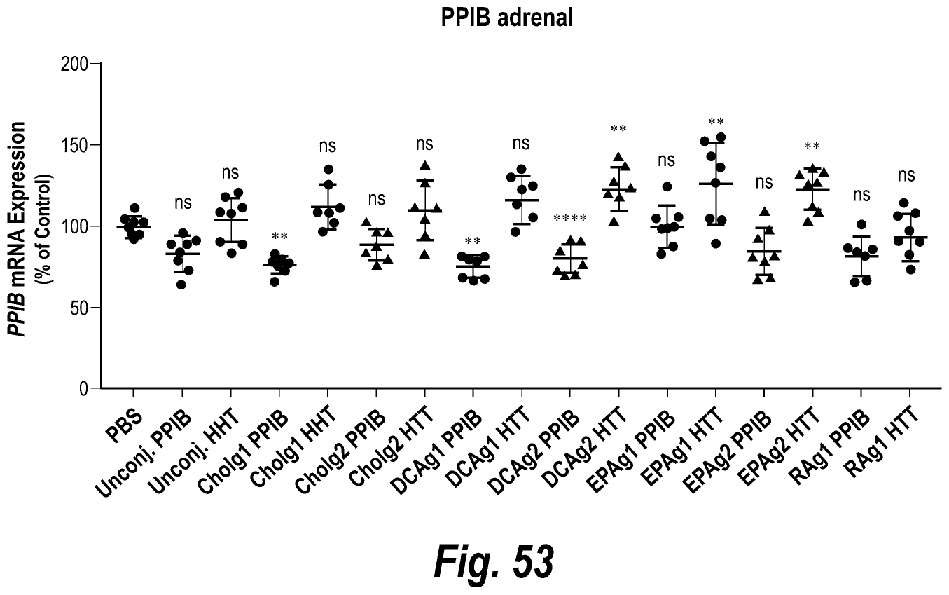

FIG. 52 depicts efficacy of cholesterol g1-conjugated htt hsiRNA, cholesterol g2-conjugated htt hsiRNA, DCA g1-conjugated htt hsiRNA, DCA g2-conjugated htt hsiRNA and RA g1-conjugated htt hsiRNA for silencing htt mRNA in the adrenal gland. FIG. 53 depicts efficacy of cholesterol g1-conjugated PPIB hsiRNA, DCA g1-conjugated PPIB hsiRNA and DCA g2-conjugated PPIB hsiRNA for silencing PPIB mRNA in the adrenal gland.

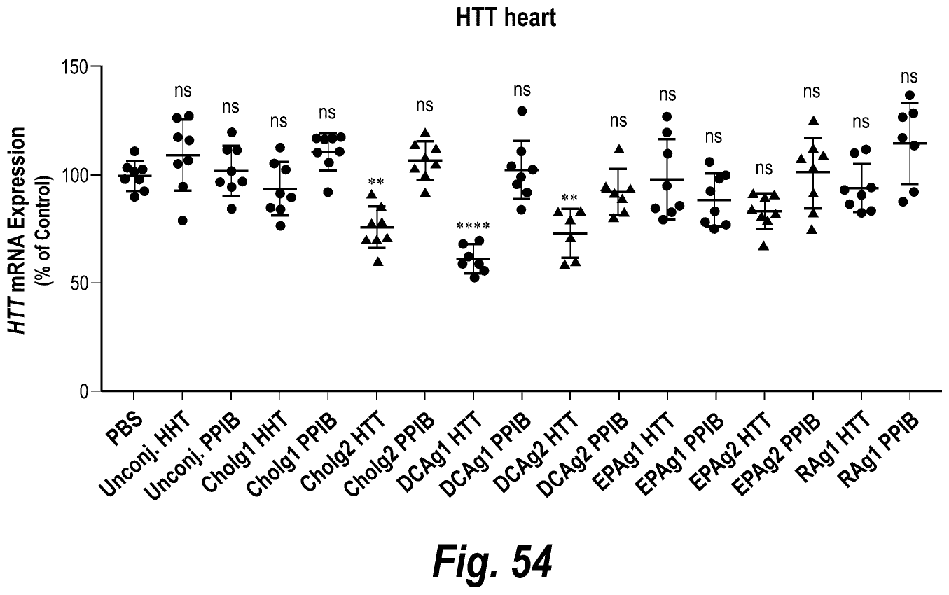

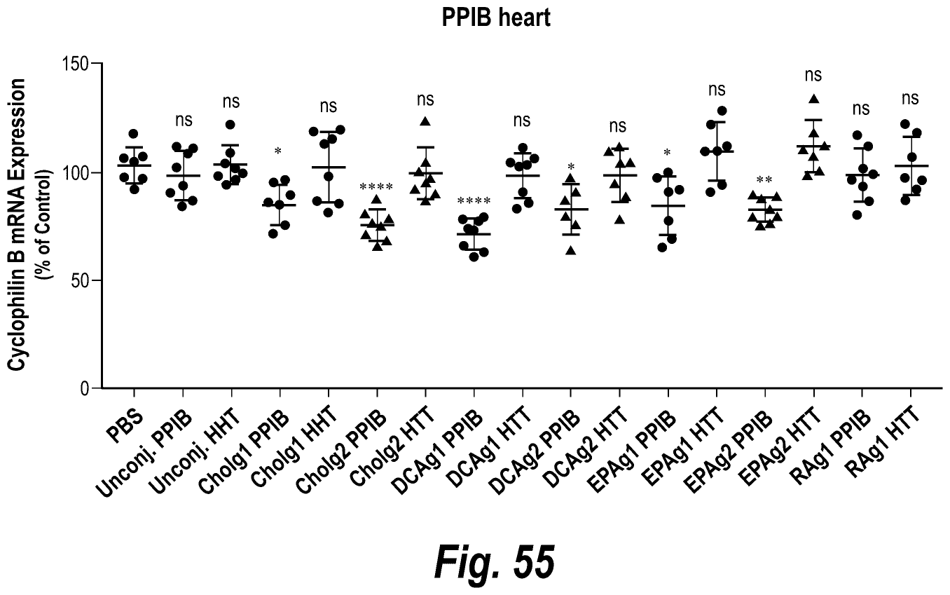

FIG. 54 depicts efficacy of cholesterol g2-conjugated htt hsiRNA, DCA g1-conjugated htt hsiRNA and DCA g2-conjugated htt hsiRNA for silencing htt mRNA in the heart. FIG. 55 depicts efficacy of cholesterol g1-conjugated PPIB hsiRNA, cholesterol g2-conjugated PPIB hsiRNA, DCA g1-conjugated PPIB hsiRNA, DCA g2-conjugated PPIB hsiRNA, EPA g1-conjugated PPIB hsiRNA and EPA g2-conjugated PPIB hsiRNA for silencing PPIB mRNA in the heart.

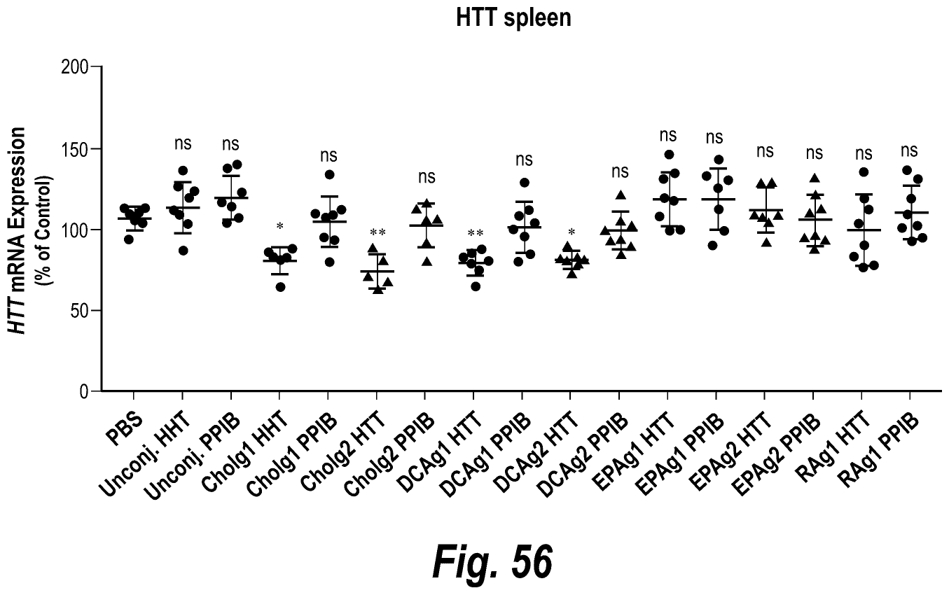

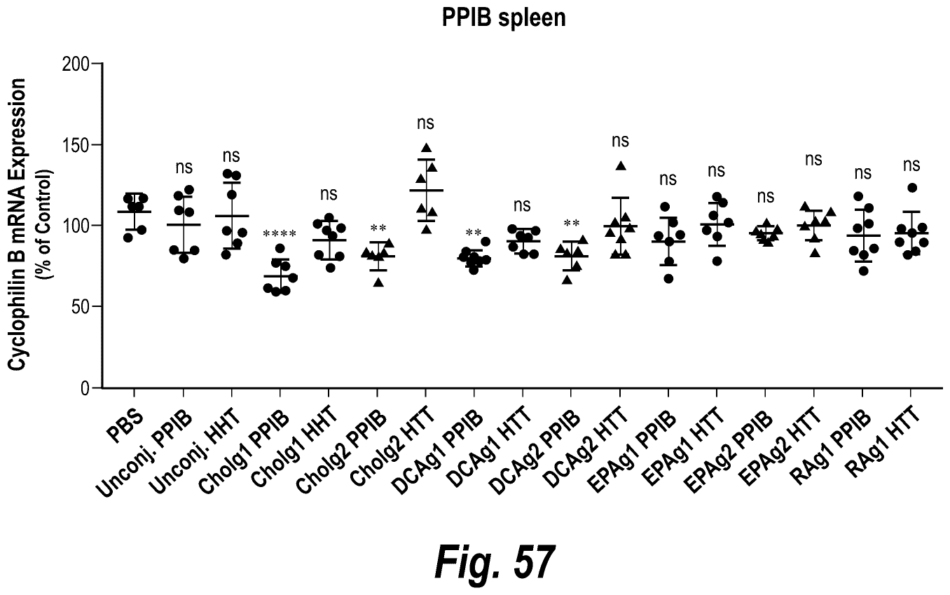

FIG. 56 depicts efficacy of cholesterol g1-conjugated htt hsiRNA, cholesterol g2-conjugated htt hsiRNA, DCA g1-conjugated htt hsiRNA and DCA g2-conjugated htt hsiRNA for silencing htt mRNA in the spleen. FIG. 57 depicts efficacy of cholesterol g1-conjugated PPIB hsiRNA, cholesterol g2-conjugated PPIB hsiRNA, DCA g1-conjugated PPIB hsiRNA and DCA g2-conjugated PPIB hsiRNA for silencing PPIB mRNA in the spleen.

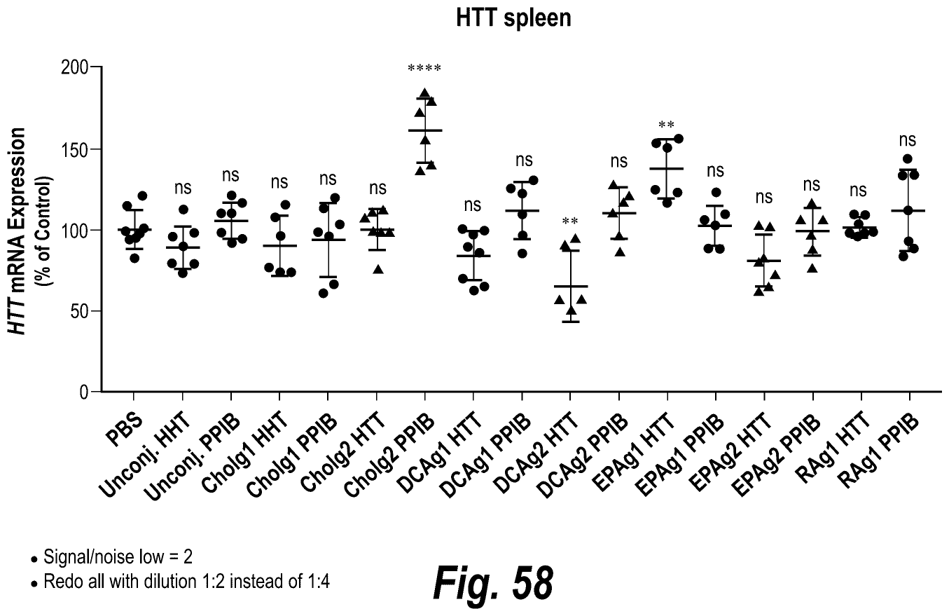

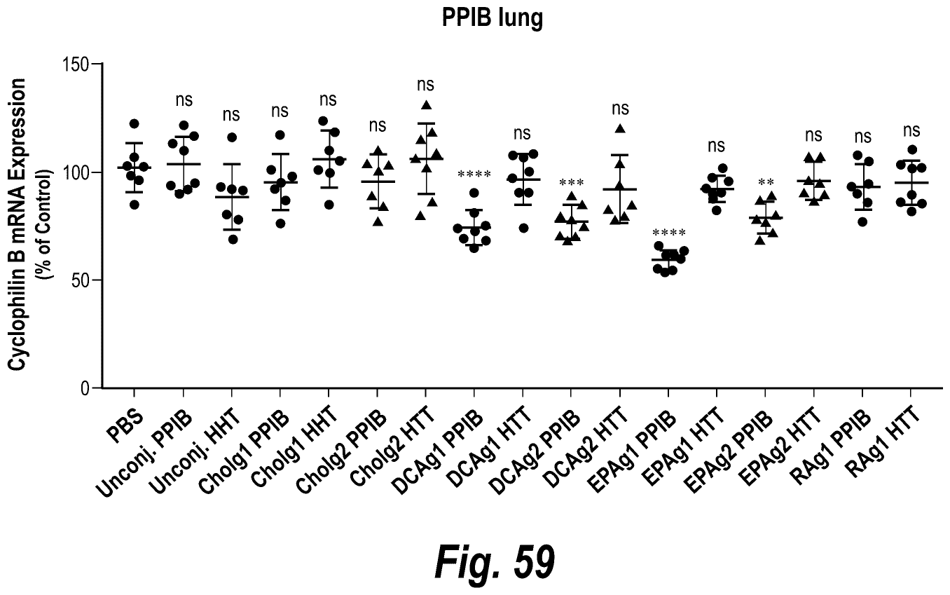

FIG. 58 depicts efficacy of DCA g2-conjugated htt hsiRNA and EPA g1-conjugated htt hsiRNA for silencing htt mRNA in the lung. FIG. 59 depicts efficacy of DCA g1-conjugated PPIB hsiRNA, DCA g2-conjugated PPIB hsiRNA, EPA g1-conjugated PPIB hsiRNA and EPA g2-conjugated PPIB hsiRNA for silencing PPIB mRNA in the lung.

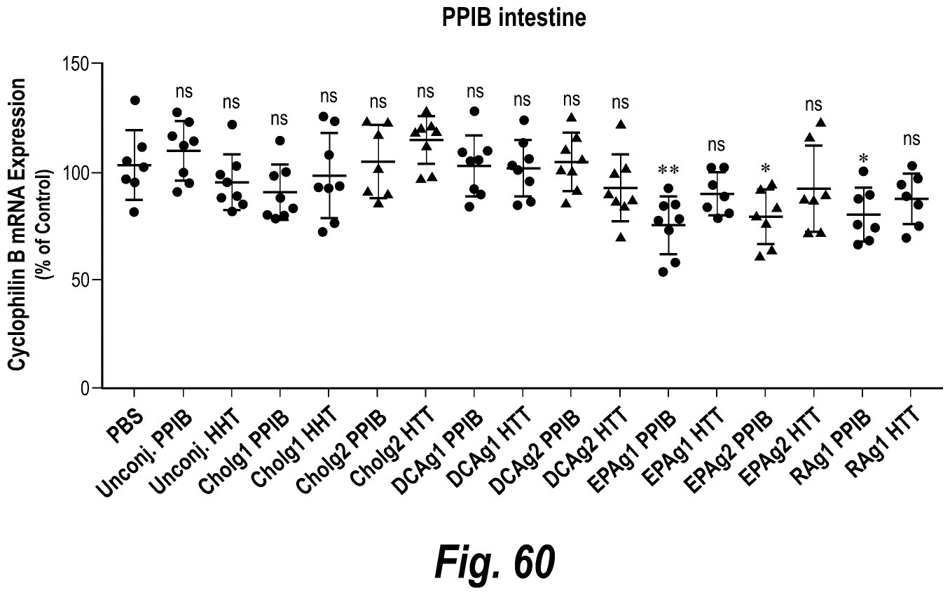

FIG. 60 depicts efficacy of EPA g1-conjugated htt hsiRNA, EPA g2-conjugated htt hsiRNA and RA g1-conjugated htt hsiRNA for silencing htt mRNA in the intestine.

Cy3-Htt HsiRNA Sequences:

| | |
|---|---|
| Sense strand | Cy3- |
| 5′-3′ | (fC)#(mA)#(fG)(mU)(fA)(mA)(fA)(mG)(fA)(mG)(fA)(mU)(fU)#(mA)#(fA)- |
| | conjugate (SEQ ID NO: 1) |
| Antisense | VP(mU)#(fU)#(mA)(fA)(mU)(fC)(mU)(fC)(mU)(fU)(mU)(fA)(mC)#(fU)# |
| strand 5′-3′ | (mG)#(fA)#(mU)#(fA)#(mU)#(fA) (SEQ ID NO: 2) |

VP = vinyl phosphonate; mU, mA, mC, mG = 2′-OMe; fU, fA, fC, fG = 2′-F; # = phosphorothioate linkage.

Cy3-PPIB HsiRNA Sequences:

| | |
|---|---|
| Sense strand | Cy3- |
| 5′-3′ | (fC)#(mA)#(fA)(mA)(fU)(mU)(fC)(mC)(fA)(mU)(fC)(mG)(fU)#(mG)#(fA)- |
| | conjugate (SEQ ID NO: 3) |
| Antisense | VP(mU)#(fC)#(mA)(fC)(mG)(fA)(mU)(fG)(mG)(fA)(mA)(fU)(mU)#(fU)# |
| strand 5′-3′ | (mG)#(fC)#(mU)#(fG)#(mU)#(fU) (SEQ ID NO: 4) |

VP = vinyl phosphonate; mU, mA, mC, mG = 2′-OMe; fU, fA, fC, fG = 2′-F; # = phosphorothioate linkage.

1-148. (canceled)

149. A compound comprising a structure of: 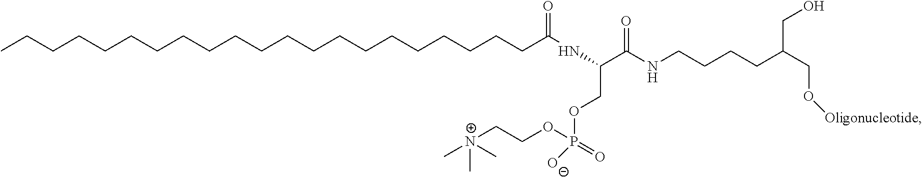
an ionized form thereof, or a salt thereof.

150. The compound of claim 149, wherein the oligonucleotide is a double-stranded nucleic acid comprising a first oligonucleotide and a second oligonucleotide, wherein:
(1) the first oligonucleotide comprises at least 16 contiguous nucleotides, a 5′ end, a 3′ end, and has complementarity to a target;
(2) the second oligonucleotide comprises at least 15 contiguous nucleotides, a 5′ end, a 3′ end, and has homology with a target; and
(3) a portion of the first oligonucleotide is complementary to a portion of the second oligonucleotide.

151. The compound of claim 150, wherein the first oligonucleotide comprises a chemical modification at a 2′-OH sugar position of each nucleotide.

152. The compound of claim 151, wherein the chemical modification at the sugar position of the nucleotides comprises a 2′-methoxy chemical modification or a 2′-fluoro modification.

153. The compound of claim 152, wherein the nucleotides at positions 2 and 14 from the end of the first oligonucleotide are not 2′-methoxy-ribonucleotides.

154. The compound of claim 150, wherein the second oligonucleotide comprises a chemical modification at a 2′-OH sugar position of each nucleotide.

155. The compound of claim 154, wherein the chemical modification at the sugar position of the nucleotides comprises a 2′-methoxy chemical modification or a 2′-fluoro modification.

156. The compound of claim 155, wherein the nucleotides at position 2 from the 5′ end of the second oligonucleotide is a 2′-methoxy-ribonucleotide.

157. The compound of claim 150, wherein the nucleotides of the first oligonucleotide are connected to adjacent nucleotides via phosphodiester or phosphorothioate linkages.

158. The compound of claim 157, wherein the nucleotides at positions 1-7 from the 3′ end of the first oligonucleotide are connected to adjacent nucleotides via phosphorothioate linkages.

159. The compound of claim 157, wherein the nucleotides at positions 1 and 2 from the end of the first nucleotide are connected to adjacent nucleotides via phosphorothioate linkages.

160. The compound of claim 150, wherein the nucleotides of the second oligonucleotide are connected to adjacent nucleotides via phosphodiester or phosphorothioate linkages.

161. The compound of claim 160, wherein the second oligonucleotide comprises a dT-dT moiety at the 3′ end.

162. The compound of claim 161, wherein the nucleotides at positions 1 and 2 from the 3′ end of the second nucleotide are connected to adjacent nucleotides via phosphorothioate linkages.

163. The compound of claim 162, wherein the nucleotides at positions 1 and 2 from the 5′ end of the second nucleotide are connected to adjacent nucleotides via phosphorothioate linkages.

164. The compound of claim 150, wherein the first oligonucleotide comprises a moiety X at the 5′ end, wherein X is selected from the group consisting of:

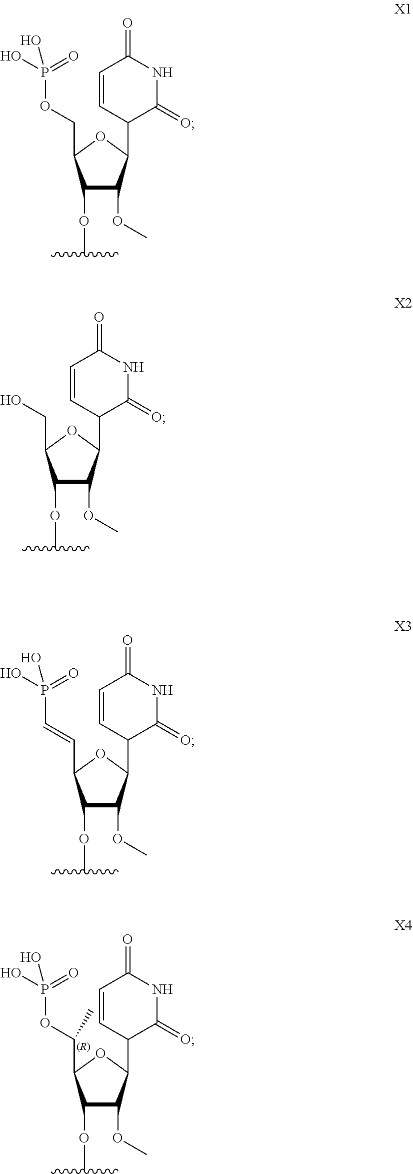

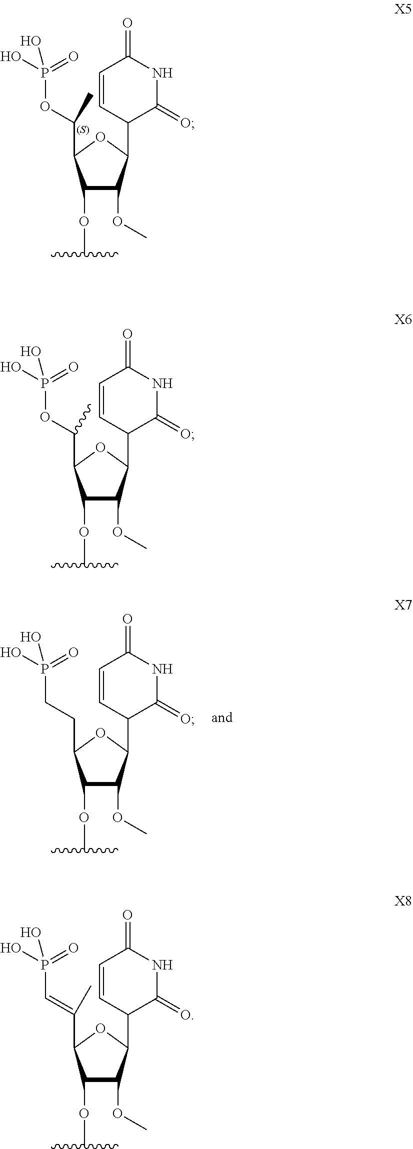

165. The compound of claim 164, wherein X is X3.

166. The compound of claim 150, wherein the target is soluble FLT.

167. A pharmaceutical composition comprising the compound of claim 149.

168. A method for treating a disease or disorder in a subject, comprising administering to the subject the compound of claim 149.

169. The method of claim 168, wherein the disease or disorder is an angiogenic disease or disorder.

170. The method of claim 169, wherein the angiogenic disease or disorder is pre-eclampsia (PE), postpartum PE, eclampsia, or HELLP.


SEQUENCE LISTING (10 sequences)

SEQ ID NO: 1 - RNA, 15 nt, synthetic construct
  misc_feature 1..15: note = Description of Artificial Sequence: Synthetic oligonucleotide
  source 1..15: mol_type = other RNA
cagtaaagagattaa

SEQ ID NO: 2 - RNA, 20 nt, synthetic construct
  misc_feature 1..20: note = Description of Artificial Sequence: Synthetic oligonucleotide
  source 1..20: mol_type = other RNA
ttaatctctttactgatata

SEQ ID NO: 3 - RNA, 15 nt, synthetic construct
  misc_feature 1..15: note = Description of Artificial Sequence: Synthetic oligonucleotide
  source 1..15: mol_type = other RNA
caaattccatcgtga

SEQ ID NO: 4 - RNA, 20 nt, synthetic construct
  misc_feature 1..20: note = Description of Artificial Sequence: Synthetic oligonucleotide
  source 1..20: mol_type = other RNA
tcacgatggaatttgctgtt

SEQ ID NO: 5 - RNA, 20 nt, synthetic construct
  misc_feature 1..20: note = Description of Artificial Sequence: Synthetic oligonucleotide
  source 1..20: mol_type = other RNA
ttaatctctttactgatata

SEQ ID NO: 6 - RNA, 20 nt, synthetic construct
  misc_feature 1..20: note = Description of Artificial Sequence: Synthetic oligonucleotide
  source 1..20: mol_type = other RNA
tcacgatggaatttgctgtt

SEQ ID NO: 7 - RNA, 15 nt, synthetic construct
  misc_feature 1..15: note = Description of Artificial Sequence: Synthetic oligonucleotide
  source 1..15: mol_type = other RNA
gcacattaaacagaa

SEQ ID NO: 8 - RNA, 20 nt, synthetic construct
  misc_feature 1..20: note = Description of Artificial Sequence: Synthetic oligonucleotide
  source 1..20: mol_type = other RNA
ttctgtttaatgtgcataaa

SEQ ID NO: 9 - RNA, 15 nt, synthetic construct
  misc_feature 1..15: note = Description of Artificial Sequence: Synthetic oligonucleotide
  source 1..15: mol_type = other RNA
tgacaaatacgatta

SEQ ID NO: 10 - RNA, 20 nt, synthetic construct
  misc_feature 1..20: note = Description of Artificial Sequence: Synthetic oligonucleotide
  source 1..20: mol_type = other RNA
taatcgtatttgtcaatcat